(19) United States
(12) Patent Application Publication
(10) Pub. No.: US 2022/0056052 A1
(43) Pub. Date: Feb. 24, 2022

(54) QUINAZOLINE DERIVATIVES AS ECTONUCLEOTIDE PYROPHOSPHATASE PHOSPHODIESTERASE 1 INHIBITORS

(71) Applicant: Riboscience LLC, Palo Alto, CA (US)

(72) Inventors: Ronald HAWLEY, Palo Alto, CA (US); Klaus KLUMPP, Palo Alto, CA (US)

(21) Appl. No.: 17/415,584

(22) PCT Filed: Dec. 27, 2019
(86) PCT No.: PCT/US2019/068669

§ 371 Date: Jun. 17, 2021

Related U.S. Application Data
(60) Provisional application No. 62/822,582, filed on Mar. 22, 2019.
(60) Provisional application No. 62/785,905, filed on Dec. 28, 2018.

(51) Int. Cl. C07F 5/02; C07F 9/6558; C07F 9/6512; C07D 239/94; C07D 239/74; C07D 401/12

(52) U.S. Cl. CPC C07F 5/025; C07F 9/65583; C07D 401/12; C07D 239/94; C07D 239/74; C07F 9/65128

(57) ABSTRACT

The present disclosure provides certain quinazoline compounds that inhibit ectonucleotide pyrophosphatase/phosphodiesterase 1 (ENPP1) enzymatic activity and are therefore useful for the treatment of diseases and conditions modulated at least in part by ENPP1. Also provided are pharmaceutical compositions containing such compounds and processes for preparing such compounds.

### CROSS-REFERENCES TO RELATED APPLICATIONS

This is an application claiming priority benefit under 35 U.S.C. § 119(e) of U.S. Provisional Application No. 62/785,905 filed Dec. 28, 2018 and Provisional Application No. 62/822,582 filed Mar. 22, 2019, the contents of each are herein incorporated by reference in their entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE DISCLOSURE

The present disclosure provides certain quinazoline compounds that inhibit ectonucleotide pyrophosphatase/phosphodiesterase 1 (ENPP1) enzymatic activity and are therefore useful for the treatment of diseases treatable by inhibition of ENPP1. Also provided are pharmaceutical compositions containing such compounds and processes for preparing such compounds.

### BACKGROUND

ENPP1 enzyme is present in a wide range of tissues and cell types, such as lymphocytes, macrophages, liver, brain, heart, kidney, vascular smooth muscle cells, and chondrocytes. ENPP1 hydrolyzes ATP and other nucleoside triphosphates and releases AMP or other nucleoside monophosphates as well as pyrophosphate (PPi) (Kato K et al. 2012 PNAS 109:16876-16881; Hessle L et al. 2002 PNAS 99:9445-9449). The enzyme can also hydrolyze other nucleoside monophosphate esters (Kato K et al. 2012 PNAS 109:16876-16881). ENPP1 has been identified as the dominant 2′-3′-cGAMP hydrolase in cultured cells, tissue extracts and blood (Li L et al. 2014 Nat Chem Biol 10:1043-1048). Tissues and blood from ENPP1 knockout mice lack 2′-3′-cGAMP hydrolase activity. Elevated levels of ENPP1 have been associated with calcific aortic valve disease (CAVD) and calcium pyrophosphate dihydrate (CPPD) disease, an inflammatory disease resulting from CPPD crystal deposits in the joint and surrounding tissues (Cote N et al. 2012 Eur J Pharmacol 689:139-146; Johnson K et al. 2001 Arthritis Rheum 44:1071). ENPP1 expression is upregulated in certain hepatocellular carcinomas, glioblastomas, melanomas, testicular, pancreatic and thyroid and breast cancers and has been associated with resistance to chemotherapy (see Lau W M et al. 2013 PLoS One 8:5; Bageritz J et al. 2014 Mol Cell Oncology 1:3; Bageritz J et al. 2014 Cell Death, Differentiation 21:929-940; Umar A et al. 2009 Mol Cell Proteomics 8:1278-1294). ENPP1 upregulation and variants of ENPP1 are also associated with insulin resistance and type 2 diabetes (Meyre D et al. 2005 Nat Genet 37:863-867; Maddux B A et al. 1995 Nature 373:448-451; Rey D et al. 2012 Mol Biol Rep 39:7687-7693) and enzyme activity of ENPP1 was reported to be required for the inhibition of insulin receptor signaling (Chin C N et al. 2009 Eur J Pharmacol 606:17-24).

Cyclic GMP-AMP synthase (cGAS) is a pattern recognition receptor that synthesizes the endogenous messenger molecule cGAMP from ATP and GTP in response to the presence of DNA derived from viruses, bacteria, damaged mitochondria or cancer cells. The cGAMP molecule then binds to the stimulator of interferon genes (STING) protein, which initiates a signaling response that activates innate immunity and results in the production of type I interferon, antiviral and immune-stimulatory cytokines (Sun L et al. 2013 Science 339:786-791; Wu J et al. 2013 Science 339:826-830; Gao D et al. 2013 Science 341:903-906; Li X et al. 2013 Science 341:1390-1394; Schoggins J W et al. 2014 Nature 505:691-695; Wassermann R et al. 2015 Cell Host Microbe 17:799-810; Watson R O et al. 2015 Cell Host Microbe 17:811-819; Collins A et al. 2015 Cell Host Microbe 17:820-828; West A et al. 2015 Nature 520:533-557; Woo S R et al. 2014 Immunity 41:830-842; Deng L et al. 2014 Immunity 41:843-852; Chen Q et al. 2016 Nat Immunol 17:1142-1148). The cGAS enzyme, cGAMP messenger and STING are is also involved in host defense against RNA viruses and the immune control of tumor development (Aguirre S et al. 2012 PLoS Pathog 8: e1002934; Barber G N 2015 Nat Rev Immunol 15:760-770). ENPP1 has been identified as the enzyme that naturally hydrolyzes cGAMP and therefore counteracts the innate immune response against infectious agents, damaged cells and cancer cells (Li L et al. 2014 Nat Chem Biol 10:1043-1048). The efficacy of non-hydrolyzable cGAMP analogs in inducing functional immune responses is higher than that of natural, hydrolysable cGAMP (Li L et al. 2014 Nat Chem Biol 10:1043-1048; Corrales L et al. 2015 Cell Rep 11:1018-1030). Virus infection has been demonstrated to be facilitated by ENPP1 overexpression and is attenuated by silencing of ENPP1 (Wang J et al. 2018 Mol Immunol 95:56-63).

Inhibitors of cGAMP hydrolysis may therefore be used to increase the effectiveness of immune responses against cancer cells and tumors and against infections by RNA or DNA viruses or bacteria. Inhibitors of ENPP1 and of cGAMP or nucleoside triphosphate hydrolysis may also be used for the treatment of inflammatory diseases that are associated with elevated nucleotidase levels, reduced nucleoside triphosphate, reduced cGAMP or reduced nucleoside monophosphate ester levels or diseases associated with elevated nucleoside or nucleoside monophosphate levels. For these reasons, ENPP1 is an attractive therapeutic target for the treatment of diseases.

The present disclosure addresses these needs and provides related advantages as well.

### SUMMARY

In a first aspect, provided is a compound of Formula (IA):

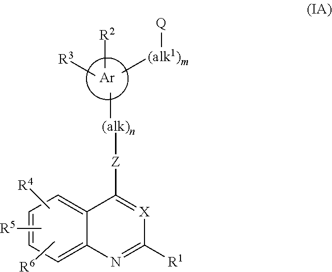

wherein:

X is N or CH;

Z is NH, O, S, SO, or SO₂;

alk is alkylene optionally substituted with one, two, or three halo;

m and n are independently 0 or 1;

(i) —Ar-(alk¹)ₘ-Q is wherein Ar is aryl, heteroaryl, cycloalkyl, or heterocyclyl;
alk¹ is alkylene wherein one carbon atom in the alkylene chain is optionally replaced by oxygen and further wherein alkylene is optionally substituted with one, two, or three halo; and
Q is —B(OH)₂ or —P(O)(R^{a})(R^{b}) wherein R^{a} and R^{b} are independently selected from hydroxy, alkoxy,-Oaryl (where aryl is optionally substituted with one to three substituents independently selected from alkyl, alkenyl, alkoxy, halo, haloalkyl, amino, alkylamino, dialkylamino, cyano, or nitro), —O—(CH₂)OCOR^{c} (where R^{c} is alkyl), —O-(alk²)OR^{d} (where alk² is alkylene and R^{d} is alkyl), —S—(CH₂)₂SCOR^{e} (where R^{e} is alkyl), or —NR^{g}—(CHR)OCOR^{f} (where R is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl either alone or as part of phenylalkyl is optionally substituted with one to three substituents independently selected from alkyl, alkoxy, halo, hydroxy, cyano or nitro), R^{f} is alkyl or benzyl, and R^{g} is hydrogen or R^{g} together with R forms —(CH₂)₃—); or R^{a} and R^{b} together with the phosphorus atom to which they are attached form a ring of formula (c):

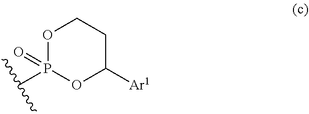

wherein Ar¹ is phenyl or six membered heteroaryl optionally substituted with one to three halo; provided that, when Q is —P(O)(R^{a})(R^{b}) or —B(OH)₂, then at least one of n and m is 1; or

(ii) —Ar-(alk¹)ₘ-Q is a ring of formula (a):

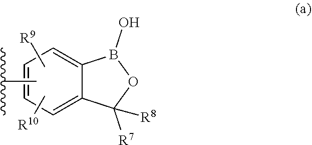

R¹ is hydrogen, alkyl, alkoxy, halo, haloalkyl, haloalkoxy, amino, alkylamino, dialkylamino, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, diaminoalkyl, diaminoalkoxy, diaminoalkylamino or cyano;

R², R³, R⁹, and R¹⁰ are independently hydrogen, alkyl, alkoxy, halo, haloalkyl, or haloalkoxy, or cyano;

R⁷ and R⁸ are independently hydrogen or alkyl; and

one of R⁴, R⁵, and R⁶ is hydrogen, alkyl, hydroxy, alkoxy, halo, haloalkyl, haloalkoxy, cyano, amino, alkylamino, or dialkylamino; and the remaining two of R⁴, R⁵, and R⁶ are independently hydrogen, alkyl, alkoxy, hydroxy, halo, haloalkyl, haloalkoxy, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano); or

a pharmaceutically acceptable salt thereof.

In a first aspect, provided is a compound of Formula (I):

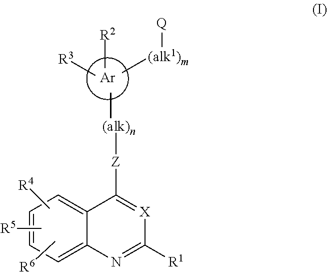

wherein:

X is N or CH;

Z is bond, NH, O, S, SO, or SO₂;

alk is alkylene optionally substituted with one, two, or three halo;

m and n are independently 0 or 1;

(i) —Ar-(alk¹)ₘ-Q is where Ar is aryl, heteroaryl, cycloalkyl, or heterocyclyl;
alk¹ is alkylene optionally substituted with one, two, or three halo; and
Q is —OSO₂NH₂, —NHSO₂NH₂, —P(O)(OH)₂, or —B(OH)₂; provided that, when Q is —P(O)(OH)₂ or —B(OH)₂, then least one of n and m is 1; or

(ii) —Ar-(alk¹)ₘ-Q is a ring of formula (a) or (b):

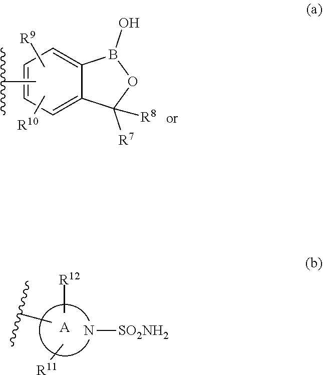

wherein ring A is piperidinyl or pyrrolidinyl,
provided that, when Z is a bond, then n is 1;

R¹, R², R³, R⁹, R¹⁰, R¹¹ and R¹² are independently hydrogen, alkyl, alkoxy, halo, haloalkyl, haloalkoxy, or cyano;

R⁷ and R⁸ are independently hydrogen or alkyl; and

one of R⁴, R⁵, and R⁶ is hydrogen, alkyl, hydroxy, alkoxy, halo, haloalkyl, haloalkoxy, cyano, amino, alkylamino, or dialkylamino; and the remaining two of R⁴, R⁵, and R⁶ are independently hydrogen, alkyl, alkoxy, hydroxy, halo, haloalkyl, haloalkoxy, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano); or

a pharmaceutically acceptable salt thereof.

In a second aspect, provided is a pharmaceutical composition comprising a compound of the present disclosure, e.g., Formula (IA) or (I) (or any of the embodiments thereof described herein) or a pharmaceutically acceptable salt thereof and a pharmaceutically acceptable excipient.

In a third aspect, provided are methods of treating a disease or condition modulated at least in part by ENPP1 in a patient, preferably in a patient recognized as needing such a treatment, comprising administering to the patient a compound of Formula (IA) or (I) (or any of the embodiments thereof described herein) or a pharmaceutically acceptable salt thereof in a therapeutically effective amount. In one embodiment, the disease is cancer such as hepatocellular carcinomas, glioblastomas, melanomas, testicular, pancreatic, thyroid and breast cancer. In another embodiment, the disease is an inflammatory disease e.g., calcific aortic valve disease and calcium pyrophosphate dihydrate. In yet another embodiment the disease metabolic disease e.g., type 2 diabetes or a viral infection.

In a fourth aspect, provided is a compound of Formula (IA) or (I) (or any embodiments thereof described herein) or a pharmaceutically acceptable salt thereof for use as a medicament. In one embodiment, the medicament is for use in the treatment of cancer such as hepatocellular carcinomas, glioblastomas, melanomas, testicular, pancreatic, thyroid and breast cancer. In another embodiment, the medicament is for use in the treatment of an inflammatory disease e.g., calcific aortic valve disease and calcium pyrophosphate dihydrate. In yet another embodiment, the medicament is for use in the treatment of a metabolic disease e.g., type 2 diabetes or a viral infection.

In a fifth aspect provided is the use of a compound of Formula (IA) or (I) or a pharmaceutically acceptable salt thereof (and any embodiments thereof disclosed herein) in the manufacture of a medicament for treating a disease in a patient in which the activity of ENPP1 contributes to the pathology and/or symptoms of the disease. In one embodiment, the disease is cancer such as hepatocellular carcinomas, glioblastomas, melanomas, testicular, pancreatic, thyroid and breast cancer. In another embodiment, the disease is an inflammatory disease e.g., calcific aortic valve disease and calcium pyrophosphate dihydrate. In yet another embodiment, the disease metabolic disease e.g., type 2 diabetes or a viral disease.

In any of the aforementioned aspects involving the treatment of cancer, are further embodiments comprising administering the compound of Formula (IA) or (I) or a pharmaceutically acceptable salt thereof (or any embodiments thereof disclosed herein) in combination with at least one additional anticancer. When combination therapy is used, the agents can be administered simultaneously or sequentially.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Amino” means a —NH₂.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen or alkyl as defined above, e.g., aminomethyl, aminoethyl, methylaminomethyl, and the like.

“Aminoalkylamino” means a —NR^{a}R^{b} radical where R^{a} is hydrogen or alkyl and R^{b} is aminoalkyl as defined above, e.g., aminoethylamino, dimethylaminoethylamino, diethylaminoethylamino, dimethylaminopropylamino, diethylaminopropylamino, and the like.

“Aminoalkyloxy” or “aminoalkoxy” means a —OR^{a} radical where R^{a} is aminoalkyl as defined above, e.g., aminoethyloxy, dimethylaminoethyloxy, diethylaminoethyloxy, dimethylaminopropyloxy, diethylaminopropyloxy, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxyalkylamino” means a —NRR′ radical where R is hydrogen or alkyl and R′ is alkoxyalkyl as defined above, e.g., methoxyethylamino, ethoxyethylamino, propoxypropylamino, ethoxypropylamino, and the like.

“Alkoxyalkyloxy” or “alkoxyalkoxy” means a —O—R radical where R is alkoxyalkyl as defined above, e.g., methoxyethoxy, ethoxyethoxy, and the like.

“Aryl” means a monovalent monocyclic or bicyclic aromatic hydrocarbon radical of 6 to 10 ring atoms e.g., phenyl or naphthyl.

“Phenyloxy” means a —OR radical where R is phenyl.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Cycloalkyloxy” means a —OR radical where R is cycloalkyl (including specific heterocyclyl rings) as defined above e.g., cyclopropyloxy, and the like.

“Carboxy” means —COOH.

“Dialkylamino” means a —NRR′ radical where R and R′ are alkyl as defined above, e.g., dimethylamino, methylethylamino, and the like.

“Diaminoalkyl” means a linear monovalent hydrocarbon radical of one to six carbon atoms or a branched monovalent hydrocarbon radical of three to six carbons substituted with two —NR′R″ where R′ and R″ are independently hydrogen or alkyl as defined above, e.g., diaminoethyl, 1,3-diaminopropyl, 2-amino-3-methylaminopropyl, and the like.

“Diaminoalkylamino” means a —NR^{a}R^{b} radical where R^{a} is hydrogen or alkyl and R^{b} is diaminoalkyl as defined above, e.g., diaminoethylamino, 1,3-diaminopropylamino, 2-amino-3-methylaminopropylamino, and the like.

“Diaminoalkyloxy” means a —OR^{a} radical where R^{a} is diaminoalkyl as defined above, e.g., 2-diaminoethyloxy, 1,3-diaminopropyloxy, 2-amino-3-methylaminopropyloxy, and the like.

“Halo” means fluoro, chloro, bromo, or iodo, preferably fluoro or chloro.

“Haloalkyl” means alkyl radical as defined above, which is substituted with one or more halogen atoms, such as one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkylamino” means a —NR^{a}R^{b} radical where R^{a} is hydrogen or alkyl and R^{b} is hydroxyalkyl as defined above, e.g., hydroxyethylamino, hydroxypropylamino, and the like.

“Hydroxyalkyloxy” or “hydroxyalkoxy” means a —OR^{a} radical where R^{a} is hydroxyoalkyl as defined above, e.g., hydroxyethyloxy, hydroxypropyloxy, and the like.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

“Heterocyclylalkyl” or “heterocycloalkyl” means a -(alkylene)-R radical where R is heterocyclyl ring (including specific heterocyclyl rings) as defined above e.g., tetraydrofuranylmethyl, piperazinylmethyl, morpholinylethyl, and the like.

“Heterocyclylamino” means a —NRR′ radical where R is hydrogen or alkyl and R′ is heterocyclyl (including specific heterocyclyl rings) as defined above.

“Heterocyclylalkylamino” or “heterocycloalkylamino” means a —NRR′ radical where R is hydrogen or alkyl and R′ is heterocyclylalkyl ring (including specific heterocyclyl rings) as defined above e.g., tetraydrofuranylmethylamino, piperazinylethylamino, morpholinylethylamino, piperidinylmethylamino, and the like.

“Heterocyclyloxy” means a —OR radical where R is heterocyclyl (including specific heterocyclyl rings) as defined above.

“Heterocyclylalkyloxy” or “heterocycloalkyloxy” means a —OR radical where R is heterocyclylalkyl ring (including specific heterocyclyl rings) as defined above e.g., tetraydrofuranylmethyloxy, piperazinylethyloxy, morpholinylethyloxy, piperidinylmethyloxy, and the like.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heteroaryloxy” means a —OR radical where R is heteroaryl (including specific heteroaryl rings) as defined above.

The present disclosure also includes protected derivatives of compounds of the present disclosure (I). For example, when compounds of the present disclosure contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of the present disclosure can be prepared by methods well known in the art.

The present disclosure also includes polymorphic forms and deuterated forms of the compound of the present disclosure and/or a pharmaceutically acceptable salt thereof.

A “pharmaceutically acceptable salt” of a compound means a salt that is pharmaceutically acceptable and that possesses the desired pharmacological activity of the parent compound. Such salts include: acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference in its entirety.

The compounds of the present disclosure may have asymmetric centers. Compounds of the present disclosure containing an asymmetrically substituted atom may be isolated in optically active or racemic forms. It is well known in the art how to prepare optically active forms, such as by resolution of materials. All chiral, diastereomeric, all mixtures of chiral or diasteromeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding (S) stereoisomer as an impurity i.e., the (S) stereoisomer in less than about 5%, preferably 2% by wt and then it is denoted as a mixture of R and S isomers, the amounts of R or S isomer in the mixture is greater than about 5%, preferably 2% w/w.

Certain compounds of the present disclosure can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all hydrates of a compound of the present disclosure are within the scope of this disclosure.

The compounds of the present disclosure may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into compounds of the present invention, such as a compound of Formula (IA) or (I) (and any embodiment thereof disclosed herein including specific compounds) include isotopes of hydrogen, carbon, nitrogen, oxygen, phosphorus, sulfur, fluorine, chlorine, and iodine such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with .sup.3H and .sup.14C) can be useful in compound or substrate tissue distribution assays. Tritiated (i.e., .sup.3H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “patient” is generally synonymous with the term “subject” and includes all mammals including humans. Examples of patients include humans, livestock such as cows, goats, sheep, pigs, and rabbits, and companion animals such as dogs, cats, and horses. Preferably, the patient is a human.

The terms “inhibiting” and “reducing,” or any variation of these terms in relation of EPPI, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of EPPI activity compared to normal.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of the present disclosure and/or a pharmaceutically acceptable salt thereof that, when administered to a patient for treating a disease, is sufficient to effect such treatment for the disease. The “therapeutically effective amount” will vary depending on the compound, the disease and its severity and the age, weight, etc., of the mammal to be treated.

Representative compound of Formula (I) are disclosed in Table 1 below:

| Cpd # | Structure | Name |
|---|---|---|
| 1 | 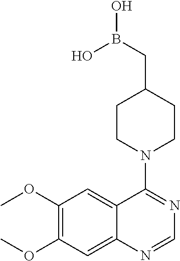 [SMILES: COc1cc2ncnc(N3CCC(CB(O)O)CC3)c2cc1OC] | ((1-(6,7-dimethoxyquinazolin-4-yl)piperidin-4- yl)methyl)boronic acid |
| 2 | 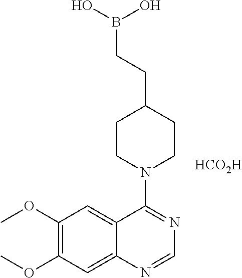 | (2-(1-(6,7-dimethoxyquinazolin-4-yl)piperidin-4- yl)ethyl)boronic acid formic acid salt |
| 3 | 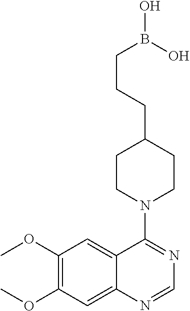 [SMILES: COc1cc2ncnc(N3CCC(CCCB(O)O)CC3)c2cc1OC] | (3-(1-(6,7-dimethoxyquinazolin-4-yl)piperidin-4- yl)propyl)boronic acid |
| 4 | 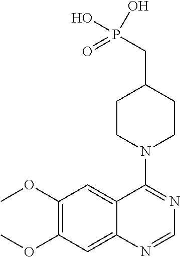 [SMILES: COc1cc2ncnc(N3CCC(CP(=O)(O)O)CC3)c2cc1OC] | ((1-(6,7-dimethoxyquinazolin-4-yl)piperidin-4- yl)methyl)phosphonic acid |
| 5 | 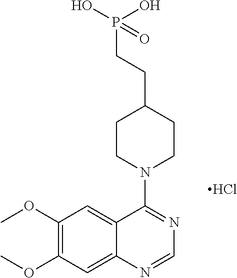 [SMILES: COc1cc2ncnc(N3CCC(CCP(=O)(O)O)CC3)c2cc1OC ; Cl] | (2-(1-(6,7-dimethoxyquinazolin-4-yl)piperidin-4- yl)ethyl)phosphonic acid hydrochloride |
| 6 | 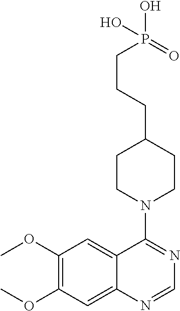 [SMILES: COc1cc2ncnc(N3CCC(CCCP(=O)(O)O)CC3)c2cc1OC] | (3-(1-(6,7-dimethoxyquinazolin-4-yl)piperidin-4- yl)propyl)phosphonic acid |
| 7 | 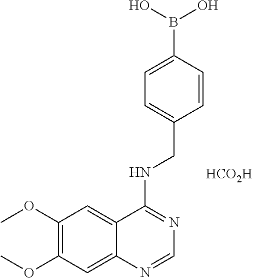 [SMILES: COc1cc2ncnc(NCc3ccc(B(O)O)cc3)c2cc1OC ; "HCO2H"] | (4-(((6,7-dimethoxyquinazolin-4- yl)amino)methyl)phenyl)boronic acid formic acid salt |
| 8 | 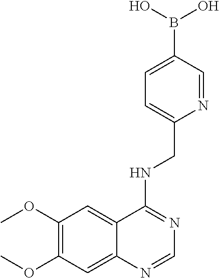 [SMILES: COc1cc2ncnc(NCc3ccc(B(O)O)cn3)c2cc1OC] | (6-(((6,7-dimethoxyquinazolin-4- yl)amino)methyl)pyridin-3-yl)boronic acid |
| 9 | 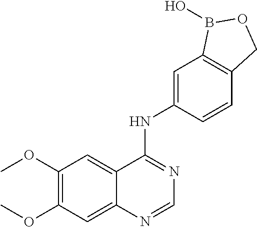 [SMILES: COc1cc2ncnc(Nc3ccc4c(c3)B(O)OC4)c2cc1OC] | 6-((6,7-dimethoxyquinazolin-4-yl)amino)-1,3- dihydro-2,1-benzoxaborol-1-ol |
| 10 | 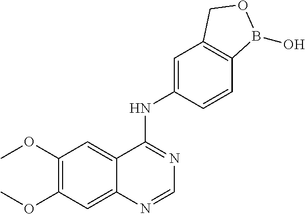 [SMILES: COc1cc2ncnc(Nc3ccc4c(c3)COB4O)c2cc1OC] | 5-((6,7-dimethoxyquinazolin-4-yl)amino)-1,3- dihydro-2,1-benzoxaborol-1-ol |
| 11 | 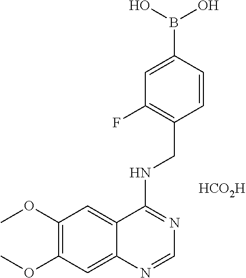 [SMILES: COc1cc2ncnc(NCc3ccc(B(O)O)cc3F)c2cc1OC ; "HCO2H"] | 4-(((6,7-dimethoxyquinazolin-4-yl)amino)methyl)- 3-fluorophenylboronic acid formic acid salt |
| 12 | 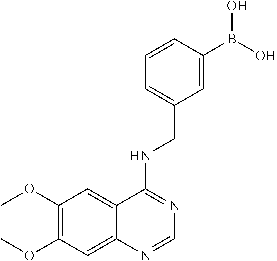 [SMILES: COc1cc2ncnc(NCc3cccc(B(O)O)c3)c2cc1OC] | (3-(((6,7-dimethoxyquinazolin-4- yl)amino)methyl)phenyl)boronic acid |
| 13 | 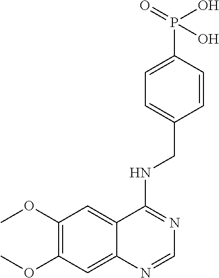 [SMILES: COc1cc2ncnc(NCc3ccc(P(=O)(O)O)cc3)c2cc1OC] | (4-(((6,7-dimethoxyquinazolin-4- yl)amino)methyl)phenyl)phosphonic acid |
| 14 | 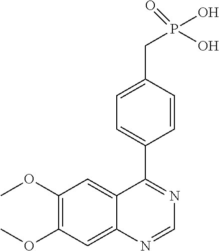 [SMILES: COc1cc2ncnc(-c3ccc(CP(=O)(O)O)cc3)c2cc1OC] | ((4-(6,7-dimethoxyquinazolin-4- yl)phenyl)methyl)phosphonic acid |
| 15 | 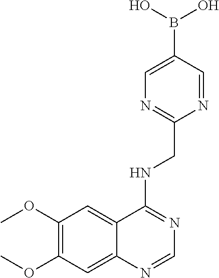 [SMILES: COc1cc2ncnc(NCc3ncc(B(O)O)cn3)c2cc1OC] | 2-((6,7-dimethoxyquinazolin-4- ylamino)methyl)pyrimidin-5-ylboronic acid |
| 16 | 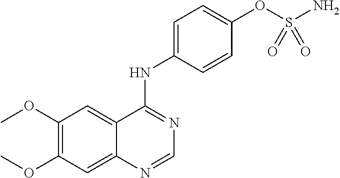 [SMILES: COc1cc2ncnc(Nc3ccc(OS(N)(=O)=O)cc3)c2cc1OC] | 4-((6,7-dimethoxyquinazolin-4-yl)amino)phenyl sulfamate |
| 17 | 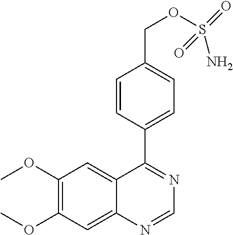 [SMILES: COc1cc2ncnc(-c3ccc(COS(N)(=O)=O)cc3)c2cc1OC] | 4-(6,7-dimethoxyquinazolin-4-yl)benzyl sulfamate |
| 18 | 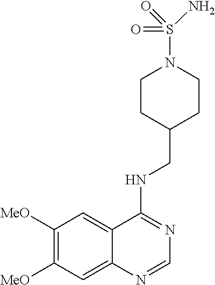 [SMILES: COc1cc2ncnc(NCC3CCN(S(N)(=O)=O)CC3)c2cc1OC] | 4-(((6,7-dimethoxyquinazolin-4- yl)amino)methyl)piperidine-1-sulfonamide |
| 19 | 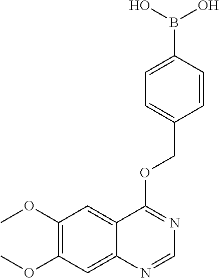 [SMILES: COc1cc2ncnc(OCc3ccc(B(O)O)cc3)c2cc1OC] | (4-(((6,7-dimethoxyquinazolin-4- yl)oxy)methyl)phenyl)boronic acid |
| 20 | 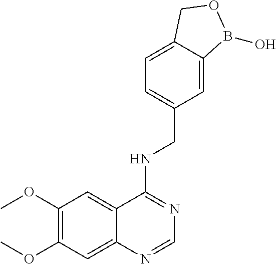 [SMILES: COc1cc2ncnc(NCc3ccc4c(c3)B(O)OC4)c2cc1OC] | 6-(((6,7-dimethoxyquinazolin-4-yl)oxy)methyl)- 3H-2,1-benzoxaborol-1-ol |
| 21 | 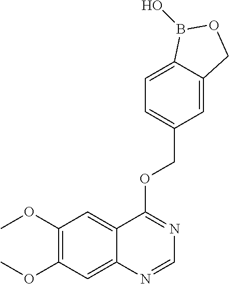 [SMILES: COc1cc2ncnc(OCc3ccc4c(c3)COB4O)c2cc1OC] | 5-(((6,7-dimethoxyquinazolin-4-yl)oxy)methyl)- 3H-2,1-benzoxaborol-1-lol |
| 22 | 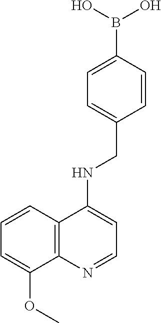 [SMILES: COc1cccc2c(NCc3ccc(B(O)O)cc3)ccnc12] | (4-(((8-methoxyqunolin-4- yl)amino)methyl)phenyl)boronic acid |
| 23 | 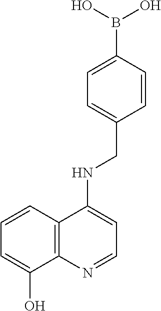 [SMILES: OB(O)c1ccc(CNc2ccnc3c(O)cccc23)cc1] | (4-(((8-hydroxyquinolin-4- yl)amino)methyl)phenyl)boronic acid |
| 24 | 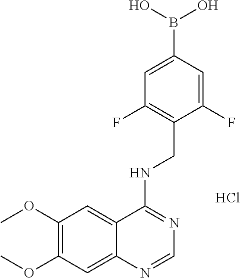 [SMILES: COc1cc2ncnc(NCc3c(F)cc(B(O)O)cc3F)c2cc1OC ; Cl] | 4-(((6,7-dimethoxyquinazolin-4-yl)amino)methyl)- 3,5-difluorophenylboronic acid hydrochloride |
| 25 | 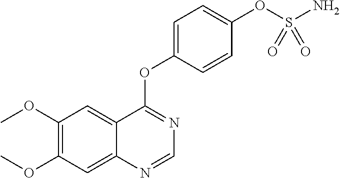 [SMILES: COc1cc2ncnc(Oc3ccc(OS(N)(=O)=O)cc3)c2cc1OC] | 4-((6,7-dimethoxyquinazolin-4-yl)oxy)phenyl sulfamate |
| 26 | 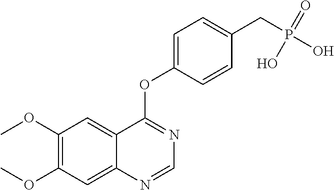 [SMILES: COc1cc2ncnc(Oc3ccc(CP(=O)(O)O)cc3)c2cc1OC] | 4-(6,7-dimethoxyquinazolin-4- yloxy)benzylphosphonic acid |
| 27 | 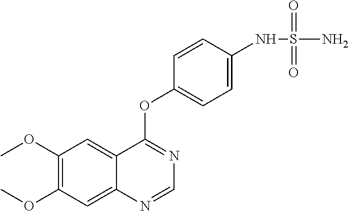 [SMILES: COc1cc2ncnc(Oc3ccc(NS(N)(=O)=O)cc3)c2cc1OC] | 4-((6,7-dimethoxyquinazolin-4-yl)oxy)phenyl sulfamide |
| 28 | 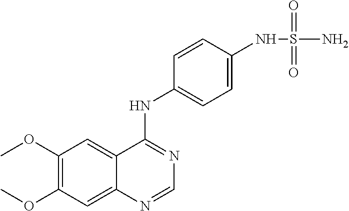 [SMILES: COc1cc2ncnc(Nc3ccc(NS(N)(=O)=O)cc3)c2cc1OC] | 4-((6,7-dimethoxyquinazolin-4-yl)amino)phenyl sulfamide |
| 29 | 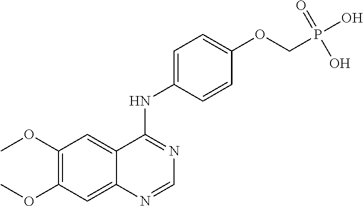 [SMILES: COc1cc2ncnc(Nc3ccc(OCP(=O)(O)O)cc3)c2cc1OC] | 4-(6,7-dimethoxyquinazolin-4- ylamino)phenoxymethylphosphonic acid |
| 30 | 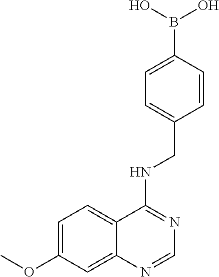 [SMILES: COc1ccc2c(NCc3ccc(B(O)O)cc3)ncnc2c1] | 4-(((7-methoxyquinazolin-4- yl)amino)methyl)phenylboronic acid |
| 31 | 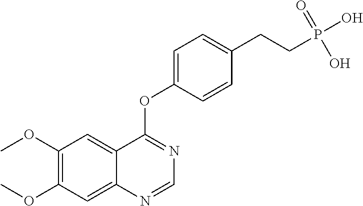 [SMILES: COc1cc2ncnc(Oc3ccc(CCP(=O)(O)O)cc3)c2cc1OC] | 4-(6,7-dimethoxyquinazolin-4- ylamino)phenethylphosphonic acid |
| 32 | 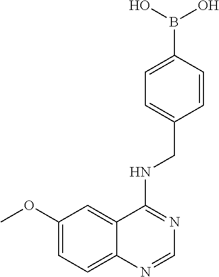 [SMILES: COc1ccc2ncnc(NCc3ccc(B(O)O)cc3)c2c1] | 4-(((6-methoxyquinazolin-4- yl)amino)methyl)phenylboronic acid |
| 33 | 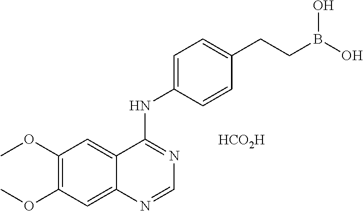 [SMILES: COc1cc2ncnc(Nc3ccc(CCB(O)O)cc3)c2cc1OC ; "HCO2H"] | 2-(4-((6,7-Dimethoxyquinazolin-4- yl)amino)phenyl)ethylboronic acid formic acid salt |
| 34 | 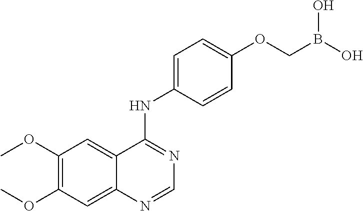 [SMILES: COc1cc2ncnc(Nc3ccc(OCB(O)O)cc3)c2cc1OC] | (4-(6,7-dimethoxyquinazolin-4- ylamino)phenyl)methylboronic acid |
| 35 | 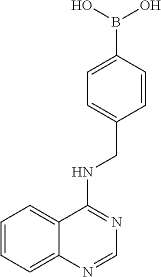 [SMILES: OB(O)c1ccc(CNc2ncnc3ccccc23)cc1] | 4-((quinazolin-4-ylamino)methyl)phenylboronic acid |
| 36 | 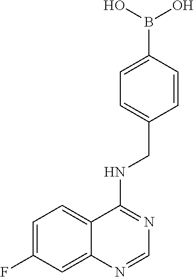 [SMILES: OB(O)c1ccc(CNc2ncnc3cc(F)ccc23)cc1] | 4-(((7-fluoroquinazolin-4- yl)amino)methyl)phenylboronic acid |
| 37 | 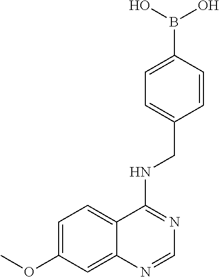 [SMILES: COc1ccc2c(NCc3ccc(B(O)O)cc3)ncnc2c1] | 4-((7-methoxyquinolin-4- ylamino)methyl)phenylboronic acid |
| 38 | 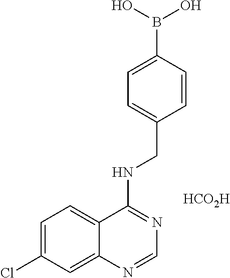 [SMILES: OB(O)c1ccc(CNc2ncnc3cc(Cl)ccc23)cc1 ; "HCO2H"] | 4-(((7-chloroquinazolin-4- yl)amino)methyl)phenylboronic acid formic acid salt |
| 39 | 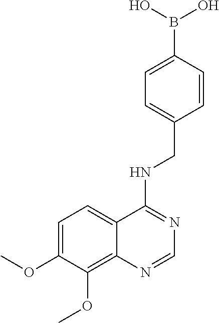 [SMILES: COc1ccc2c(NCc3ccc(B(O)O)cc3)ncnc2c1OC] | 4-(((7,8-dimethoxyquinazolin-4- yl)amino)methyl)phenylboronic acid |
| 40 | 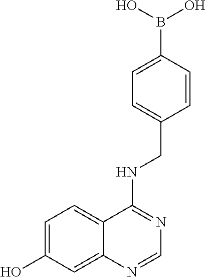 [SMILES: OB(O)c1ccc(CNc2ncnc3cc(O)ccc23)cc1] | 4-(((7-hydroxyquinazolin-4- yl)amino)methyl)penylboronic acid |
| 41 | 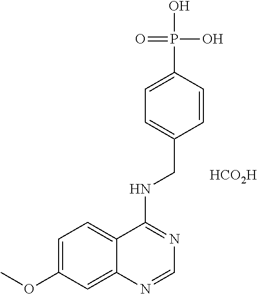 [SMILES: COc1ccc2c(NCc3ccc(P(=O)(O)O)cc3)ncnc2c1 ; "HCO2H"] | 4-(((7-methoxyquinazolin-4- yl)amino)methyl)phenylphosphonic acid formic acid salt |
| 42 | 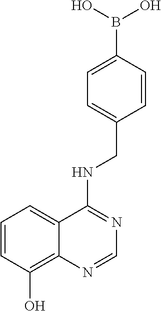 [SMILES: OB(O)c1ccc(CNc2ncnc3c(O)cccc23)cc1] | 4-(((8-hydroxyquinazolin-4- yl)amino)methyl)phenylboronic acid |
| 43 | 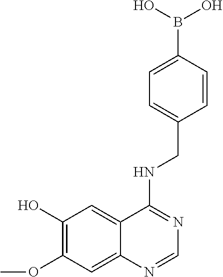 [SMILES: COc1cc2ncnc(NCc3ccc(B(O)O)cc3)c2cc1O] | 4-(((6-hydroxy-7-methoxyquinazolin-4- yl)amino)methyl)phenylboronic acid |
| 44 | 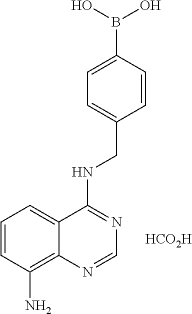 [SMILES: Nc1cccc2c(NCc3ccc(B(O)O)cc3)ncnc12 ; "HCO2H"] | 4-(((8-aminoquinazolin-4- yl)amino)methyl)phenylboronic acid formic acid salt |
| 45 | 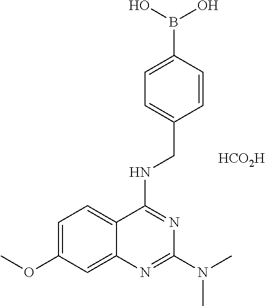 | 4-(((2-(dimethylamino)-7-methoxyquinazolin-4- yl)amino)methyl)phenylboronic acid formic acid salt |
| 46 | 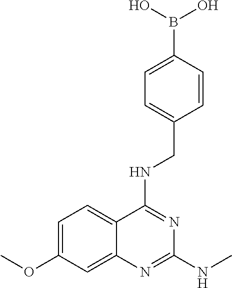 | 4-(((7-methoxy-2-(methylamino)quinazolin-4- yl)amino)methyl)phenylboronic acid |
| 47 | 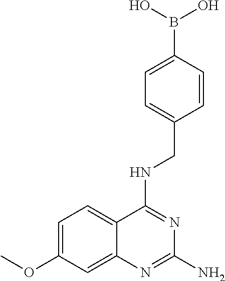 [SMILES: COc1ccc2c(NCc3ccc(B(O)O)cc3)nc(N)nc2c1] | 4-(((2-amino-7-methoxyquinazolin-4- yl)amino)methyl)phenylboronic acid |
| 48 | 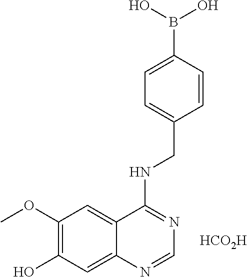 [SMILES: COc1cc2c(NCc3ccc(B(O)O)cc3)ncnc2cc1O ; "HCO2H"] | 4-(((7-hydroxy-6-methoxyquinazolin-4- yl)amino)methyl)phenylboronic acid formic acid salt |

### EMBODIMENTS

### Embodiment A1

In embodiment A1, the compounds of Formula (IA) or a pharmaceutically acceptable salt thereof as defined in the Summary above.

### Embodiment A

In embodiment A, the compounds of Formula (I) or a pharmaceutically acceptable salt thereof as defined in the Summary above.

### Embodiment B1

In embodiment B1, the compounds of embodiment A1 or a pharmaceutically acceptable salt thereof has a structure of formula (Ib1), or (Ic1):

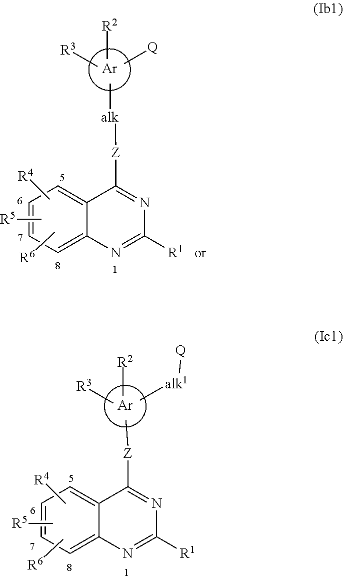

wherein Z is NH or O.

(B1i) In subembodiment (Bi) of embodiment B1, the compound or a pharmaceutically acceptable salt thereof has structure (Ib1).

(B1ii) In subembodiment (Bii) of embodiment B1, the compound or a pharmaceutically acceptable salt thereof has structure (Ic1).

(B1iii) In subembodiment (B1iii) the compounds of embodiments A1, B1, (B1i) and (B1ii) or a pharmaceutically acceptable salt thereof are those wherein Z is NH.

(B1iv) In subembodiment (B1iv), the compounds of embodiments A1, B1, (B1i) and (B1ii) or a pharmaceutically acceptable salt thereof are those wherein Z is O.

### Embodiment B

In embodiment B, the compounds of embodiment A or a pharmaceutically acceptable salt thereof has a structure of formula (Ia), (Ib), or (Ic):

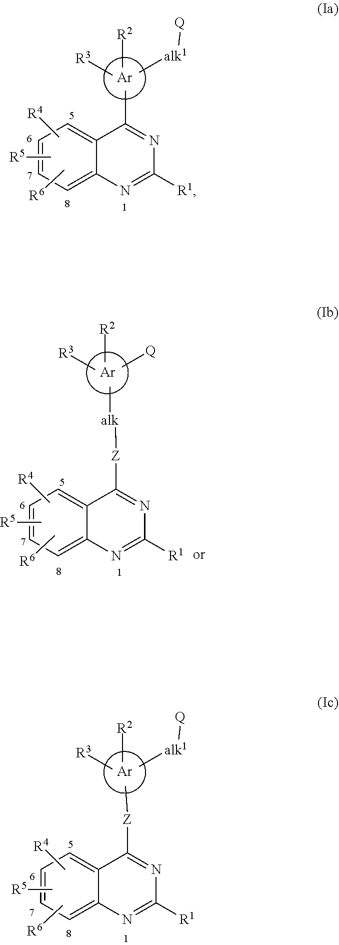

wherein Z is NH or O.

(Bi) In subembodiment (Bi) of embodiment B, the compound or a pharmaceutically acceptable salt thereof has structure (Ia).

(Bii) In subembodiment (Bii) of embodiment B, the compound or a pharmaceutically acceptable salt thereof has structure (Ib).

(Biii) In subembodiment (Biii) of embodiment B, the compound or a pharmaceutically acceptable salt thereof has structure (Ic).

(Biv) In subembodiment (Biv) the compounds of embodiments A, B, (Bii) and (Biii) or a pharmaceutically acceptable salt thereof are those wherein Z is NH.

(By) In subembodiment (By), the compounds of embodiments A, B, (Bii) and (Biii) or a pharmaceutically acceptable salt thereof are those wherein Z is O.

### Embodiment C

In embodiment C, the compounds of any one of Embodiments A1, A, B1, and B, and subembodiments contained within embodiment B1 and B, or a pharmaceutically acceptable salt thereof, are those wherein Ar is aryl or heteroaryl.

(Ci). In subembodiment Ci of embodiment C, the compounds of embodiment C or a pharmaceutically acceptable salt thereof are those wherein Ar is phenyl. In one subembodiment of subembodiment Ci, the compounds of subembodiment Ci or a pharmaceutically acceptable salt thereof is wherein Q is attached to carbon of the phenyl ring that is meta to the carbon attaching the phenyl ring to the remainder of the compound of Formula (IA) or (I). In another subembodiment of subembodiment Ci, the compounds of subembodiment Ci or a pharmaceutically acceptable salt thereof is wherein Q is attached to carbon on the phenyl ring that is para to the carbon attaching the phenyl ring to the remainder of the compound of Formula (IA), (I), (Ib1), (Ib), (Ic1) or (Ic).

(Cii). In subembodiment Cii of embodiment C, the compounds of embodiment C are those wherein Ar is heteroaryl. In one subembodiment of subembodiment Cii, the compounds of subembodiment Cii or a pharmaceutically acceptable salt thereof is wherein Ar is pyridinyl, pyrimidinyl, pyridazinyl, thienyl, furanyl, thiazolyl, oxazolyl, isoxazolyl, pyrazolyl, triazolyl, oxadiazolyl, or imidazolyl. In another subembodiment of embodiment Cii, the compounds of subembodiment Cii or a pharmaceutically acceptable salt thereof is wherein Ar is a six-membered ring such as pyridinyl, pyrimidinyl, or pyridazinyl wherein Q is attached to carbon on the pyridinyl, pyrimidinyl, or pyridazinyl ring that is meta to the carbon attaching the pyridinyl, pyrimidinyl, or pyridazinyl ring to remaining compound of Formula (I), (Ib1), (Ib), (Ic1), or (Ic).

Within subembodiment Cii, in yet another group of compounds Ar is benzofuranyl, quinolinyl, quinazolinyl, benzimidazolyl, indazolyl, benzotriazolyl, or benzoxazolyl.

### Embodiment D

In embodiment D, the compounds of Embodiments A1, A, B1, and B and subembodiments contained within embodiment B1 and B, are those wherein Ar is heterocyclyl.

(Di). In subembodiment Di of embodiment D, the compounds of subembodiment Di or a pharmaceutically acceptable salt thereof is wherein Ar is pyrrolidinyl, piperidinyl, homopiperidinyl, or piperazinyl, preferably Ar is piperidinyl.

(Dii). In subembodiment Dii of embodiment D, the compounds of subembodiment Di or a pharmaceutically acceptable salt thereof is wherein Ar is piperidinyl, homopiperidinyl, or piperazinyl wherein Q is attached to the ring atom of piperidinyl, homopiperidinyl, or piperazinyl that is meta or para, preferably para, to the ring atom attaching the aforementioned rings to the remaining compound of Formula (I), (Ib1), (1b), (Ic1) or (1c).

### Embodiment E

In embodiment E, the compounds of anyone of Embodiments A1, A, formulae (Ib1) and (Ib), and formulae (Ic1) and (Ic) in embodiments B1i, B1ii, B1iii, B1iv, Bii, Biii, Biv and Bv are those wherein (a) —Ar-Q in (Ib1) and (Ib) and Ar-alk¹-Q in (Ic1) and (Ic) is a ring of formula (a) and (b) —Ar-alk¹-Q in (Ic) is a ring of formula (b):

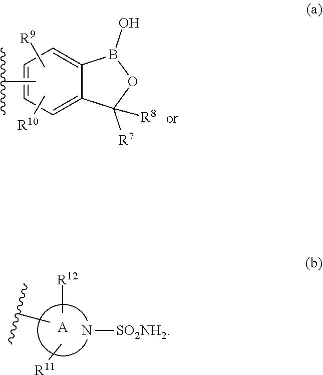

(Ei) In subembodiment Ei of embodiment E, the compounds of embodiment E or a pharmaceutically acceptable salt thereof are those wherein —Ar-Q in (Ib1) and (Ib) and Ar-alk¹-Q in (Id) and (Ic) is a ring of formula (a) wherein R⁷ and R⁸ are independently hydrogen or methyl.

(Eii) In subembodiment Eii of embodiment E, the compounds of embodiment E or a pharmaceutically acceptable salt thereof are those wherein —Ar-Q in (Ib1) and (Ib) and —Ar-alk¹-Q in (Ic1) and (Ic) is a ring of formula (a) wherein R⁷ and R⁸ are hydrogen.

(Eiii) In subembodiment Eiii of embodiment E, the compounds of embodiment E or a pharmaceutically acceptable salt thereof are those wherein —Ar-Q in (Ib1) and (Ib) and —Ar-alk¹-Q in (Id) and (Ic) is a ring of formula (a) wherein one of Wand R^{g} is hydrogen and other methyl, or both R⁷ and R⁸ are methyl.

(Eiv). In subembodiment Eiv of embodiment E, the compounds of embodiment E or a pharmaceutically acceptable salt thereof are those wherein —Ar-Q in (Ib) and —Ar-alk¹-Q in (Ic) is a ring of formula (b) wherein ring A is piperidinyl or pyrrolidinyl.

### Embodiment F

In embodiment F, the compounds of any one of embodiments A1, A, B1, B, C, and D and subembodiments contained therein or a pharmaceutically acceptable salt thereof are those wherein Q is —P(O)(OH)₂, —B(OH)₂ or —OSO₂NH₂.

(Fi). In subembodiment Fi of embodiment F, the compounds in embodiment F or a pharmaceutically acceptable salt thereof are those wherein Q is —P(O)(OH)₂

(Fii). In subembodiment Fii, the compounds in embodiment F or a pharmaceutically acceptable salt thereof those wherein Q is —B(OH)₂.

(Fiii). In subembodiment Fiii, the compounds in embodiments A1, B1, C, and D and subembodiments contained therein or a pharmaceutically acceptable salt thereof those wherein Q is —P(O)(R^{a})(R^{b}). Within subembodiment (Fiii), in a first group of compounds, Q is R^{a} and R^{b} are independently selected from hydroxy, alkoxy,-Oaryl (where aryl is optionally substituted with one to three substituents independently selected from alkyl, halo, haloalkyl, cyano, or nitro), —O—(CH₂)OCOR^{c} (where R^{c} is alkyl), —O-(alk²)OR^{d} (where alk² is alkylene and R^{d} is alkyl), and —S—(CH₂)₂SCOR^{e} (where R^{e} is alkyl). Preferably, R^{a} and R^{b} are independently selected from alkoxy, —Oaryl (where aryl is optionally substituted with one to three substituents independently selected from alkyl, halo, haloalkyl, cyano, or nitro), —O—(CH₂)OCOR^{c} (where R^{c} is alkyl), and —O-(alk²)OR^{d} (where alk² is alkylene and R^{d} is alkyl, such as methyl, isopropyl, n-propyl, isobutyl, n-butyl). Preferably, R^{a} and R^{b} are independently hydroxy, alkoxy,-Ophenyl (where phenyl is optionally substituted with one to three substituents independently selected from alkoxy, halo, haloalkyl, cyano, or nitro), —O—(CH₂)OCOR^{c} (where R^{c} is alkyl), or —NH—(CHR)OCOR^{f} (where R is alkyl, R^{f} is alkyl such as methyl, isopropyl, n-propyl, isobutyl, n-butyl or benzyl), preferably hydroxy or alkoxy.

Within subembodiment (Fiii), in a second group of compounds, Q is IV is selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, or nitro), —O—(CH₂)OCOR^{c} (where R^{c} is alkyl), —O-(alk²)OR^{d} (where alk² is alkylene and R^{d} is alkyl), and —S—(CH₂)₂SCOR^{e} (where R^{e} is alkyl), and R^{b} is selected from —NR^{g}—(CHR)OCOR^{f} (where R is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl either alone or as part of phenylalkyl is optionally substituted with one to three substituents independently selected from alkyl, alkoxy, halo, hydroxy, cyano or nitro), R^{f} is alkyl or benzyl, and R^{g} is hydrogen or together with R forms —(CH₂)₃—), Preferably, R^{a} is selected from alkoxy and —Oaryl (where aryl is optionally substituted with one to three substituents independently selected from halo, cyano, or nitro) and R^{b} is selected from —NR^{g}—(CHR)OCOR^{f} (where R is alkyl such as methyl, isopropyl, n-propyl, isobutyl, n-butyl).

Within subembodiment (Fiii), in a third group of compounds, Q is where r R^{a} and R^{b} together with the phosphorus atom to which they are attached form a ring of formula (i):

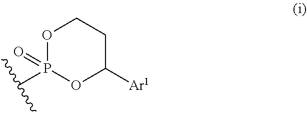

wherein Ar¹ is phenyl or six membered heteroaryl optionally substituted with one to three halo. Preferably, Ar¹ is phenyl substituted one to three halo or pyridinyl.

### Embodiment G1

In embodiment G1, the compounds of any one of embodiments A1, B1, C, D, and F and subembodiments contained therein or a pharmaceutically acceptable salt thereof are those wherein one of n and m is 1 and the other of n and m is 0. In a first subembodiment of embodiment G1, alk or alk1 is independently methyl, ethyl, or propyl, preferably methyl when Ar is a six membered ring. In a second subembodiment of embodiment G1, n is 0, m is 1, and alk1 is —O—CH₂— or —O—(CH₂)₂— when Ar is a six membered ring wherein O in —O—CH₂— and —O—(CH₂)₂— is attached to Ar.

### Embodiment G

In embodiment G, the compounds of any one of embodiments A, B, C, D, and F and subembodiments contained therein or a pharmaceutically acceptable salt thereof are those wherein alk and alk¹ are independently methyl, ethyl, or propyl, preferably methyl when Ar is a six membered ring.

### Embodiment H

In embodiment H, the compounds of embodiment A has a structure of formula (Id):

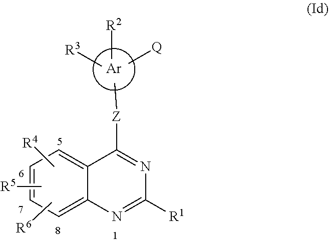

wherein:
Z is NH or O; Q is —OSO₂NH₂ and Ar is aryl or heteroaryl.

(Hi). In subembodiment Hi of embodiment H, the compounds of embodiment H or a pharmaceutically acceptable salt thereof are those wherein Ar is phenyl. In one subembodiment of embodiment Hi, the compounds of embodiment H or a pharmaceutically acceptable salt thereof are those wherein Q is attached to carbon on the phenyl ring that is meta to the carbon attaching the phenyl ring to the remainder of the compound of Formula (I). In another subembodiment of embodiment Hi, the compounds of embodiment H or a pharmaceutically acceptable salt thereof are those wherein Q is attached to carbon on the phenyl ring that is para to the carbon attaching the phenyl ring to the remainder of the compound of Formula (I).

(Hii). In subembodiment Hii of embodiment H, the compounds of embodiment H or a pharmaceutically acceptable salt thereof are those wherein Ar is heteroaryl. With subembodiment Hiii, in one group of compounds or a pharmaceutically acceptable salt thereof Ar is pyridinyl, pyrimidinyl, thiazolyl, oxazolyl, isoxazolyl, pyrazolyl, triazolyl, oxadiazolyl or pyridazinyl. In another subembodiment of embodiment Hii, Ar is pyridinyl, pyrimidinyl, thiazolyl, oxazolyl, isoxazolyl, pyrazolyl, triazolyl, oxadiazolyl or pyridazinyl wherein the Q is attached to carbon atom of the pyridinyl, pyrimidinyl, thiazolyl, oxazolyl, isoxazolyl, pyrazolyl, triazolyl, oxadiazolyl or pyridazinyl ring that is meta or para to the carbon attaching the pyridinyl, pyrimidinyl, thiazolyl, oxazolyl, isoxazolyl, pyrazolyl, triazolyl, oxadiazolyl or pyridazinyl ring to remaining compound of Formula (I).

With subembodiment Hii, in yet another group of compounds Ar is benzofuranyl, quinolinyl, quinazolinyl, benzimidazolyl, indazolyl, benzotriazolyl, or benzoxazolyl.

### Embodiment I

(Ii) In (Ii) of embodiment I, the compounds of embodiment H and subembodiments contained within embodiment H or a pharmaceutically acceptable thereof are those wherein Z is NH.

(Iii) In (Iii) of embodiment I the compounds of embodiment H and subembodiments contained within embodiment H or a pharmaceutically acceptable thereof are those wherein Z is O.

### Embodiment J1

(i) In embodiment (i) of embodiment J1, the compounds of any one of embodiments A1, B1, C, D, E, F, and G1 and subembodiments contained therein or a pharmaceutically acceptable thereof are those wherein R¹ is hydrogen or alkyl, preferably hydrogen or methyl, more preferably hydrogen.

(ii) In embodiment (ii) of embodiment J1, the compounds of any one of embodiments A1, B1, C, D, E, F, and G1 and subembodiments contained therein or a pharmaceutically acceptable thereof are those wherein R¹ is halo, haloalkyl, or haloalkoxy.

(iii) In embodiment (iii) of embodiment J1, the compounds of any one of embodiments A1, B1, C, D, E, F, and G1 and subembodiments contained therein or a pharmaceutically acceptable thereof are those wherein R¹ is amino, alkylamino, or dialkylamino, preferably amino, methylamino or dimethylamino.

(iv) In embodiment (iv) of embodiment J1, the compounds of any one of embodiments A1, B1, C, D, E, F, and G1 and subembodiments contained therein or a pharmaceutically acceptable thereof are those wherein R¹ is hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, diaminoalkyl, diaminoalkoxy, diaminoalkylamino, or cyano.

### Embodiment J

In embodiment, the compounds of any one of embodiments A, B, C, D, E, F, G, H, and I and subembodiments contained therein or a pharmaceutically acceptable thereof are those wherein R¹ is hydrogen, methyl, methoxy, fluoro, trifluoromethyl, or trifluoromethoxy. In a subembodiment of embodiment J, R¹ is hydrogen, methyl, methoxy, fluoro, trifluoromethyl, or trifluoromethoxy, preferably R¹ is hydrogen or methyl, more preferably R¹ is hydrogen.

### Embodiment K

In embodiment K, the compounds of any one of embodiments A1, A, B1, B, C, D, E, F, G1, G, H, I, J1 and J and subembodiments contained therein or a pharmaceutically acceptable thereof are those wherein R², R³, R⁹, R¹⁰, R¹¹ and R¹² are independently hydrogen, methyl, ethyl, methoxy, fluoro, trifluoromethyl, trifluoromethoxy, or cyano, preferably hydrogen.

### Embodiment L

In embodiment L, the compounds of any one of embodiments A1, A, B1, B, C, D, E, F, G1, G, H, I, J1, J and K and subembodiments contained therein or a pharmaceutically acceptable thereof are those wherein R⁴ is hydrogen, alkyl, hydroxy, alkoxy, halo, haloalkyl, haloalkoxy, or cyano.

(Li) In sub embodiment Li of embodiment L, R⁴ is hydrogen, methyl, hydroxy, methoxy, ethoxy, fluoro, chloro, trifluoromethyl, cyano, or trifluoromethyl; preferably R⁴ is hydrogen, methoxy or ethoxy, more preferably R⁴ is hydrogen.

### Embodiment M

(Mi) In embodiment Mi, the compounds of any one of embodiments A1, A, B1, B, C, D, E, F, G1, G, H, I, J1, J, K and L and subembodiments contained therein or a pharmaceutically acceptable thereof are those wherein R⁵ and R⁶ are independently hydrogen, alkyl, alkoxy, hydroxy, halo, haloalkyl, or haloalkoxy; preferably R⁵ and R⁶ are independently hydrogen, alkoxy, or hydroxy, more preferably R⁵ and R⁶ are independently alkoxy such as methoxy, ethoxy, or propoxy and are attached to C6 and C7 carbons of the bicyclic ring. It is understood that the nitrogen atom para to ring carbon attached to —Z-(alk)ₙ-Ar-(alk¹)ₘ-Q group of the bicyclic ring is position 1. Certain ring numberings for the bicyclic ring are also shown in subformulae (Ib1) and (Ic1).

(Mii) In embodiment Mii, the compounds of any one of embodiments A1, A, B1, AB, C, D, E, F, G1, G, H, I, J1, J, K and L and subembodiments contained therein or a pharmaceutically acceptable thereof are those wherein

R⁵ is hydrogen, alkyl, alkoxy, hydroxy, halo, haloalkyl, or haloalkoxy; and

R⁶ is hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano).

In a first subembodiment of embodiment Mii, R⁵ is hydrogen, methoxy, ethoxy, or hydroxy, preferably R⁵ is methoxy or ethoxy; and R⁶ is 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropyl amino, 2-aminoethylamino, 2-methyl aminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, or methoxy) and one of R⁵ and R⁶ is attached to C6 and the other of R⁵ and R⁶ is attached to C7 carbons of the bicyclic ring.

(Miii) In embodiment Mi, the compounds of any one of embodiments A1, A, B1, B, C, D, E, F, G1, G, H, I, J1, J, K and L and subembodiments contained therein or a pharmaceutically acceptable thereof are those wherein R⁵ and R⁶ are independently hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl of phenyloxy and heteroaryl of heteroaryloxy are optionally substituted with one, two, or three substituents where two of the optional substituents are independently selected from alkyl, hydroxy, alkoxy, halo, haloalkyl, haloalkoxy, and cyano).

In a first subembodiment of embodiment Miii, R⁵ and R⁶ are independently 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethyl amino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethyl amino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, or methoxy) and R⁵ and R⁶ are attached to C6 and C7 carbons of the bicyclic ring.

(Miv) In embodiment Miv, the compounds of any one of embodiments A1, A, B1, B, C, D, E, F, G1, G, H, I, J1, J, K and L and subembodiments contained therein or a pharmaceutically acceptable thereof are those wherein R⁵ is hydrogen and R⁶ is hydrogen, alkyl, alkoxy, hydroxy, halo, haloalkyl, or haloalkoxy; preferably R⁶ is hydrogen, alkoxy, or hydroxy, more preferably R⁶ is independently alkoxy such as methoxy, ethoxy, or propoxy and is attached to C7 carbons of the bicyclic ring.

### Embodiment N

In embodiment N, the compounds of embodiment A1 and A those wherein X is CH. With embodiment N, the compound or a pharmaceutically acceptable thereof are those wherein Z, alk, n, m, R¹, R², R³, R⁴, R⁵, R⁶, R⁷, R⁸ R⁹, R¹⁰, R¹¹ and R¹² are as defined in embodiment B1 and B-M above, and subembodiments contained therein.

### General Synthetic Scheme

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) where Z is bond, Ar is aryl or heteroaryl, n is 0, m is 1, Q is —OSO₂NH₂, —NHSO₂NH₂, —P(O)(OH)₂, or —B(OH)₂ and other groups are as defined in the Summary can be prepared as illustrated and described in Scheme 1 below.

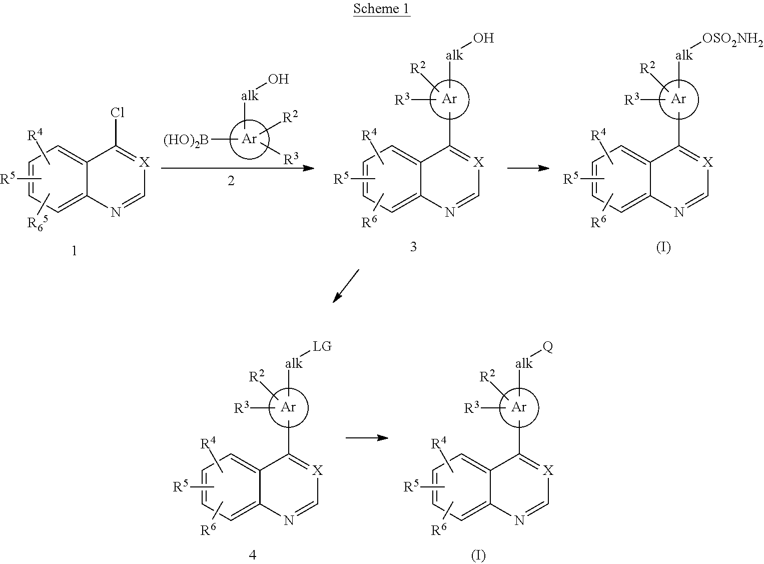

Substituting the chlorine atom in a compound of formula 1 where Z is N or CH, by reaction with a boronic acids of the formula 2 where Ar is aryl or heteroaryl under Suzuki reaction (Suzuki, A Journal of Organometallic Chemistry. 576: 147-168, and references cited therein) conditions provides an alcohol compound of formula 3. The reaction is carried out under palladium or nickel catalyzed conditions using a base such as lithium, sodium, potassium or cesium carbonate; lithium, sodium or potassium tert-butoxide; lithium, sodium or potassium hydroxide; phosphate bases such as tripotassium phosphate; or any other organic or inorganic base, in solvents comprised of a mixture of water and organic solvents such as 1,4-dioxane, tetrahydrofuran (THF), diethyl ether, toluene, ethanol or methanol, dimethylformamide (DMF) and the like, either at room temperature or heating. Compounds of formula 1 such as 4-chloro-6,7-dimethoxyquinazoline and 4-chloro-8-methoxyquinoline are commercially available.

Compounds of the Formula (I) where Q is —OSO₂NH₂ can be prepared by treatment of compounds of the formula 3 with sulfamoyl chloride in solvents such as N,N-diemthylacetamide, DMF, methylene chloride, and the like, either at room temperature or with heating in the presence or absence of a base such as triethylamine (TEA), diisopropylethylamine, imidazole, and the like.

Compounds of the Formula (I) may also be obtained by converting the hydroxyl group of the compounds of formula 3 into leaving groups, and the leaving groups displaced by nucleophiles. Conversion of the hydroxyl of formula 3 to a leaving group in compounds of formula 4 that is halide may be accomplished by means of the Appel reaction (Appel, R Angewandte Chemie International Edition in English. 14:801-811) by treatment of compound 3 with a halogenating agent such as N-bromosuccinimide, carbon tetrachloride, carbon tetrabromide, bromine, methyl iodide or iodine, in the presence of triphenylphosphine. The halo group in compounds of the formula 4 may be displaced by a variety of nucleophiles to provide a compound of Formula (I). For example, treatment of compound 4 with triethyl phosphite via heating in the absence or presence of aprotic organic solvents such as DMF or THF, followed by hydrolysis of the resulting triethylphosphonate provides a compound of Formula (I) where Q is —P(O)(OH)₂. Triethylphosphonate may be hydrolyzed in the presence of bromo- or chloro-trimethylsilane in dichloromethane, or hydrogen chloride in water, or trimethylsilyl iodide in dichloromethane either at room temperature or with heating. Compounds of Formula (I) where Q is —B(OH)₂ can be prepared by treatment of compound 4 with 4,4,5,5-tetramethyl-2-(4,4,5,5-tetramethyl-1,3,2-dioxaborolan-2-yl)-1,3,2-dioxaborolane, followed by hydrolysis of resulting 4-(4-((4,4,5,5-tetramethyl-1,3,2-dioxaborolanyl group by methods well known in the art.

Compounds of Formula (I) where Z is NH or O, Ar is aryl or heteroaryl, Q is —OSO₂NH₂, —NHSO₂NH₂, —P(O)(OH)₂, or —B(OH)₂ and other groups are as defined in the Summary can be prepared as illustrated and described in Scheme 2 below.

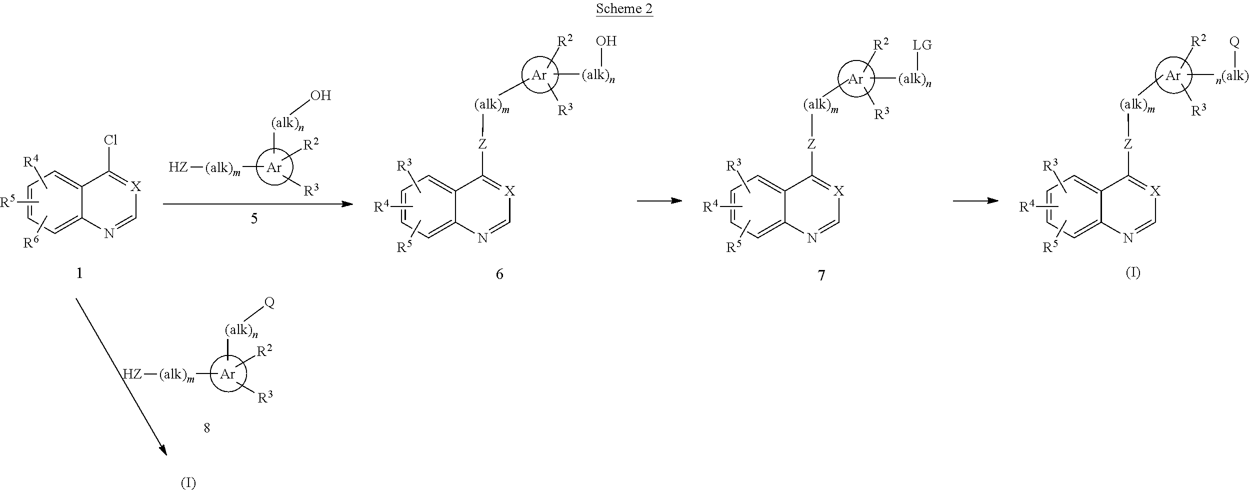

Treatment of a compound of formula 1 with a compound of formula 5 where Z is N or O and Ar, m, n, R² and R³ are as defined in the Summary of a precursor group thereof, in the presence of carbonate, hydroxide, or alkoxide (e.g. tert-butoxide) bases, either with heating or at room temperature, provides a compound of formula 6. Compounds of the formula 5 are either commercially available or may be prepared by methods well known in the art. Compounds of formula 6 can then be converted to compounds of Formula (I) as described in Scheme 1 above.

Alternatively, compounds of the Formula (I) may be prepared by displacement of the chloro in compounds of the formula 1 by compounds of the formula 8. Compounds of the formula 8 are either commercially available or can be readily prepared by methods well known in the art. The reaction is carried out by treating a mixture of compounds of the formulas 1 and 8 with carbonate, hydroxide, or alkoxide (e.g. tert-butoxide) bases, or other organic or inorganic bases, in solvents such as acetonitrile, DMF, or THF and the like, either at room temperature or with heating. Compounds of formula 8 such as (4-(aminomethyl)phenyl)boronic acid, (6-(aminomethyl)pyridin-3-yl)boronic acid, 5-aminobenzo(c)(1,2)oxaborol-1(3H)-ol, 6-aminobenzo(c)(1,2)oxaborol-1(3H)-ol, (4-(aminomethyl)-3-fluorophenyl)boronic acid, and (3-(aminomethyl)phenyl)boronic acid are commercially available.

Proceeding as above but replacing compound 5 with a compound of formula

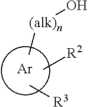

where Ar is pyrrolidine or piperidine provides compounds of the Formula 6 where Z is bond, m is 0 and Ar is pyrrolidinyl or piperidinyl ring, which can then be covered to a compound of Formula (I) as described above.

### Testing

The EPP1 inhibitory activity of the compounds of the present disclosure can be tested using the in vitro and in vivo assays described in Biological Examples 1 and 2 below.

### Administration and Pharmaceutical Composition

In general, the compounds of this disclosure will be administered in a therapeutically effective amount by any of the accepted modes of administration for agents that serve similar utilities. Therapeutically effective amounts of compounds this disclosure may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this disclosure in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

The compounds of this disclosure may be used in combination with one or more other drugs in the treatment of diseases or conditions for which compounds of this disclosure or the other drugs may have utility. Such other drug(s) may be administered, by a route and in an amount commonly used therefore, contemporaneously or sequentially with a compound of the present disclosure. When a compound of this disclosure is used contemporaneously with one or more other drugs, a pharmaceutical composition in unit dosage form containing such other drugs and the compound of the present disclosure is preferred. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly. Accordingly, the pharmaceutical compositions of the present disclosure also include those that contain one or more other drugs, in addition to a compound of the present disclosure.

The above combinations include combinations of a compound of this disclosure not only with one other drug, but also with two or more other active drugs. Likewise, a compound of this disclosure may be used in combination with other drugs that are used in the prevention, treatment, control, amelioration, or reduction of risk of the diseases or conditions for which a compound of this disclosure is useful. Such other drugs may be administered, by a route and in an amount commonly used therefore, contemporaneously or sequentially with a compound of the present disclosure. When a compound of this disclosure is used contemporaneously with one or more other drugs, a pharmaceutical composition containing such other drugs in addition to the compound of this disclosure can be used. Accordingly, the pharmaceutical compositions of the present disclosure also include those that also contain one or more other active ingredients, in addition to a compound of this disclosure. The weight ratio of the compound of this disclosure to the second active ingredient may be varied and will depend upon the effective dose of each ingredient. Generally, an effective dose of each will be used.

Where the subject in need is suffering from or at risk of suffering from cancer, the subject can be treated with a compound of this disclosure in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec™), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with a compound of this disclosure include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; antibodies (e.g., rituxan); MET inhibitor such as foretinib, carbozantinib, or crizotinib; VEGFR inhibitor such as sunitinib, sorafenib, regorafinib, lenvatinib, vandetanib, carbozantinib, axitinib; EGFR inhibitor such as afatinib, brivanib, carbozatinib, erlotinib, gefitinib, neratinib, lapatinib; PI3K inhibitor such as XL147, XL765, BKM120 (buparlisib), GDC-0941, BYL719, IPI145, BAY80-6946. BEX235 (dactolisib), CAL101 (idelalisib), GSK2636771, TG100-115; MTOR inhibitor such as rapamycin (sirolimus), temsirolimus, everolimus, XL388, XL765, AZD2013, PF04691502, PKI-587, BEZ235, GDC0349; MEK inhibitor such as AZD6244, trametinib, PD184352, pimasertinib, GDC-0973, AZD8330; and proteasome inhibitor such as carfilzomib, MLN9708, delanzomib, or bortezomib.

Other anti-cancer agents that can be employed in combination with a compound of this disclosure include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or Ril2), interferon alfa-2a; interferon alfa-2b; interferon alfa-n1; interferon alfa-n3; interferon beta-la; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a compound of the disclosure such as 8-(3-(4-acryloylpiperazin-1-yl)propyl)-6-(2,6-dichloro-3,5-dimethoxyphenyl)-2-(methylamino)pyrido(2,3-d)pyrimidin-7(8H)-one used to determine the anti-tumor activity in HGS and RT4 tumor models (Example 4 below: In HGS model, vehicle dosed group reached tumor size 645 dosing at day 42 after inoculation whereas for animals treated with 20/kg of compound, the tumor size was 55 mm3 showing significant antitumor activity and induced tumor regression), include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; Bfgf inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+-42-iethylstilbe cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a compound of this disclosure include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., cytarabine), purine analogs (e.g., mercaptopurine, thioguanine, pentostatin).

Examples of natural products useful in combination with a compound of this disclosure include but are not limited to vinca alkaloids (e.g., vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination a compound of this disclosure) include, but are not limited to, nitrogen mustards (e.g., mechloroethamine, cyclophosphamide, chlorambucil, melphalan, etc.), ethylenimine and methylmelamines (e.g., hexamethylmelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, cytarabine), purine analogs (e.g., mercaptopurine, thioguanine, pentostatin.

Examples of hormones and antagonists useful in combination a compound of this disclosure include, but are not limited to, adrenocorticosteroids (e.g., prednisone), progestins (e.g., hydroxyprogesterone caproate, megestrol acetate, medroxyprogesterone acetate), estrogens (e.g., -43-iethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an irreversible Btk inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Further examples of anti-cancer agents for use in combination with a compound of this disclosure include immune checkpoint inhibitors. Exemplary immune checkpoint inhibitors include inhibitors (smack molecules or biologics) against immune checkpoint molecules such as CD27, CD28, CD40, CD122, CD96, CD73, CD39, CD47, OX40, GITR, CSF1R, JAK, PI3K delta, PI3K gamma, TAM kinase, arginase, CD137 (also known as 4-1BB), ICOS, A2AR, A2BR, HIF-2α, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MEDI0562 or, INCAGN01949, GSK2831781, GSK-3174998, MOXR-0916, PF-04518600 or LAG525. In some embodiments, the OX40L fusion protein is MEDI6383

### EXAMPLES

The following preparations of compounds of Formula (I) are given to enable those skilled in the art to more clearly understand and to practice the present disclosure. They should not be considered as limiting the scope of the disclosure, but merely as being illustrative and representative thereof.

All solvents used were commercially available and were used without further purification. Reactions were typically run using anhydrous solvents under an inert atmosphere of nitrogen.

¹H spectra were recorded at 400 MHz or 300 MHz for proton on a Bruker 400 NMR Spectrometer equipped with a Bruker 400 BBO probe or Bruker BBFO ULTRASHIELD™ 300 AVANCE III, respectively. All deuterated solvents contained typically 0.03% to 0.05% v/v tetramethylsilane, which was used as the reference signal (set at d 0.00 for both ¹H and ¹³C).

LCMS analyses were performed on a SHIMADZU LCMS consisting of an UFLC 20-AD and LCMS 2020 MS detector. The Diode Array Detector was scanned from 190-400 nm. The mass spectrometer was equipped with an electrospray ion source (ESI) operated in a positive or negative mode. The mass spectrometer was scanned between m/z 90-900 with a scan time from 0.5 to 3.0 s.

HPLC analyses were performed on a SHIMADZU UFLC with two LC20 AD pump and a SPD-M20A Photodiiode Array Detector. The column used was an)(Bridge C18, 3.5 μm, 4.6×100 mm. A linear gradient was applied, starting at 90% A (A: 0.05% TFA in water) and ending at 95% B (B: 0.05% TFA in MeCN) over 10 min with a total run time of 15 min. The column temperature was at 40° C. with the flow rate of 1.5 mL/min. The Diode Array Detector was scanned from 200-400 nm.

Thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of 12 to 10 g silica gel and thoroughly mixing), ninhydrin (available commercially from Aldrich), or Magic Stain (generated by thoroughly mixing 25 g (NH₄)₆Mo₇O₂₄.4H₂O, 5 g (NH₄)₂Ce(IV)(NO₃)₆ in 450 mL water and 50 mL concentrated H₂SO₄) to visualize the compound. Flash chromatography was performed using 40-63 μm (230-400 mesh) silica gel from Silicycle following analogous techniques to those disclosed in Still, W. C.; Kahn, M.; and Mitra, M. Journal of Organic Chemistry, 1978, 43, 2923. Typical solvents used for flash chromatography or thin layer chromatography were mixtures of chloroform/methanol, dichloromethane/methanol, ethyl acetate/methanol and hexanes/ethyl acetate.

### SYNTHETIC EXAMPLES

### Example 1

### Synthesis of ((1-(6,7-dimethoxyquinazolin-4-yl)piperidin-4-yl)methyl)boronic Acid

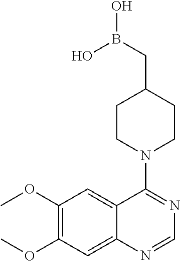 [SMILES: COc1cc2ncnc(N3CCC(CB(O)O)CC3)c2cc1OC]

### Step 1: (1-(6,7-dimethoxyquinazolin-4-yl)piperidin-4-yl)methanol

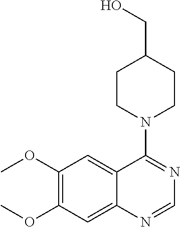 [SMILES: COc1cc2ncnc(N3CCC(CO)CC3)c2cc1OC]

To a stirred solution of 4-chloro-6,7-dimethoxyquinazoline (1.00 g, 4.452 mmol, 1.00 equiv) in acetonitrile (40 mL) were added (piperidin-4-yl)methanol (0.77 g, 6.677 mmol, 1.50 equiv) and potassium carbonate (1.23 g, 8.903 mmol, 2.00 equiv). After stirring at 80° C. for 16 h, the reaction mixture was concentrated under vacuum to remove the solvent. The residue was diluted with water and extracted with ethyl acetate. The combined organic layers were washed with saturated sodium chloride solution, dried over anhydrous sodium sulfate and filtered. The filtrate was concentrated under reduced pressure. The residue was purified by silica gel column chromatography (DCM/MeOH (20:1)) to afford 1.10 g (81%) of (1-(6,7-dimethoxyquinazolin-4-yl)piperidin-4-yl)methanol as an off-white solid.

### Step 2: 4-(4-(bromomethyl)piperidin-1-yl)-6,7-dimethoxyquinazoline

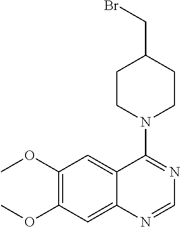 [SMILES: COc1cc2ncnc(N3CCC(CBr)CC3)c2cc1OC]

To a stirred solution of (1-(6,7-dimethoxyquinazolin-4-yl)piperidin-4-yl)methanol (600 mg, 1.978 mmol, 1.00 equiv) in DCM (25 mL) were added PPh₃ (1.56 g, 5.934 mmol, 3.00 equiv) and NBS (1.06 g, 5.934 mmol, 3.00 equiv) dropwise at 0° C. under argon atmosphere. The resulting mixture was stirred for 5 h at room temperature and then concentrated under reduced pressure. The residue was purified by silica gel column chromatography (DCM/MeOH (95:5)) to afford 450 mg (62%) of 4-(4-(bromomethyl)-piperidin-1-yl)-6,7-dimethoxyquinazoline as a brown solid.

### Step 3: 6,7-dimethoxy-4-(4-((4,4,5,5-tetramethyl-1,3,2-dioxaborolan-2-yl)methyl)piperidin-1-yl)quinazoline

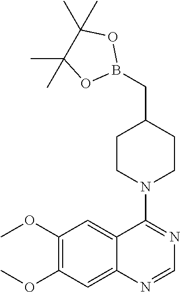 [SMILES: COc1cc2ncnc(N3CCC(CB4OC(C)(C)C(C)(C)O4)CC3)c2cc1OC]

To a stirred solution of 4-(4-(bromomethyl)piperidin-1-yl)-6,7-dimethoxyquinazoline (300 mg, 0.819 mmol, 1.00 equiv) in DMF (20 mL) were added 4,4,5,5-tetramethyl-2-(4,4,5,5-tetramethyl-1,3,2-dioxaborolan-2-yl)-1,3,2-dioxaborolane (416 mg, 1.638 mmol, 2.00 equiv), CuI (16 mg, 0.082 mmol, 0.1 equiv), PPh₃ (26 mg, 0.098 mmol, 0.12 equiv) and LiOMe (133 mg, 2.457 mmol, 3 equiv) at room temperature under argon atmosphere. After stirring at 35° C. for 18 h, the reaction mixture was diluted with water and extracted with EA. The combined organic layers were washed with saturated sodium chloride solution, dried over anhydrous sodium sulfate and filtered. The filtrate was concentrated under reduced pressure. The residue was purified by CombiFlash with following conditions: Column: C18, 120 g; Mobile Phase A: water, Mobile Phase B: acetonitrile; Flow rate: 60 mL/min; Gradient: 40% B to 70% B in 30 min, 220 & 254 nm. The fractions containing the desired product were combined and lyophilized to give 110 mg (32%) of 6,7-dimethoxy-4-(4-((4,4,5,5-tetramethyl-1,3,2-dioxaborolan-2-yl)methyl)piperidin-1-yl)quinazoline as a brown solid.

### Step 4: ((1-(6,7-dimethoxyquinazolin-4-yl)piperidin-4-yl)methyl)boronic Acid

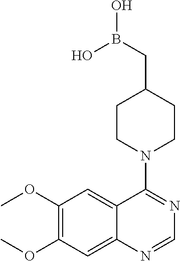 [SMILES: COc1cc2ncnc(N3CCC(CB(O)O)CC3)c2cc1OC]

To a stirred solution of 6,7-dimethoxy-4-(4-((4,4,5,5-tetramethyl-1,3,2-dioxaborolan-2-yl)methyl)piperidin-1-yl)quinazoline (110 mg, 0.266 mmol, 1.00 equiv) in water (2 mL) and THF (8 mL) was added NaIO₄ (171 mg, 0.798 mmol, 3.00 equiv). After stirring at room temperature for 5 min, 2 NHCl (0.5 mL) was added. The resulting mixture was stirred overnight and quenched with MeOH (2 mL). The mixture was concentrated under reduced pressure. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 10% B to 40% B in 7 min, 220 & 254 nm. The fractions containing the desired product were combined and lyophilized to give 27.2 mg of ((1-(6,7-dimethoxyquinazolin-4-yl)piperidin-4-yl)methyl)boronic acid as an off-white solid. MS (ESI, pos. ion) m/z: 332.3 (M+1). ¹H-NMR: (300 MHz, DMSO-d₆, ppm) δ 8.51 (s, 1H), 7.49 (s, 2H), 7.19 (s, 1H), 7.10 (s, 1H), 4.14 (d, J=12.9 Hz, 2H), 3.96-3.90 (m, 6H), 3.02 (t, J=12.0 Hz, 2H), 1.81-1.72 (m, 3H), 1.38-1.26 (m, 2H), 0.67-0.65 (m, 2H).

### Example 2

### Synthesis of (2-(1-(6,7-dimethoxyquinazolin-4-yl)piperidin-4-yl)ethyl)boronic Acid Formic Acid Salt

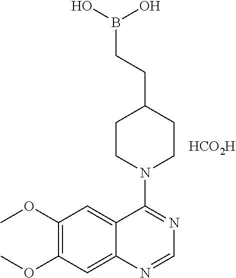

### Step 1: 2-(1-(6,7-dimethoxyquinazolin-4-yl)piperidin-4-yl)ethanol

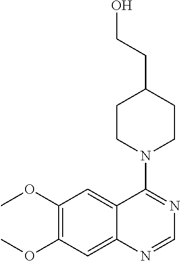 [SMILES: COc1cc2ncnc(N3CCC(CCO)CC3)c2cc1OC]

The title compound was synthesized by the same method as described in Step 1, Example 1, except 2-(piperidin-4-yl)ethan-1-ol (431.37 mg, 3.339 mmol, 1.50 equiv) was used in place of (piperidin-4-yl)methanol. Yield: 600 mg (80%).

### Step 2: 4-(4-(2-bromoethyl)piperidin-1-yl)-6,7-dimethoxyquinazoline

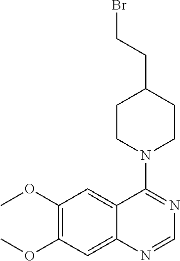 [SMILES: COc1cc2ncnc(N3CCC(CCBr)CC3)c2cc1OC]

The title compound was synthesized by the same method as described at Step 2, Example 1, except 2-(1-(6,7-dimethoxyquinazolin-4-yl)piperidin-4-yl)ethanol (650 mg, 1.95 mmol) was used in place of (1-(6,7-dimethoxyquinazolin-4-yl)piperidin-4-yl)methanol. Yield: 530 mg (68%).

### Step 3: 6,7-dimethoxy-4-(4-(2-(4,4,5,5-tetramethyl-1,3,2-dioxaborolan-2-yl)ethyl)piperidin-1-yl)quinazoline

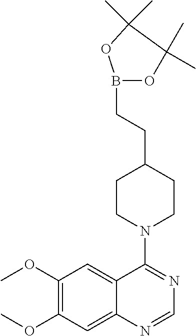

The title compound was synthesized by the same method as described at Step 3, in Example 1, except 4-(4-(2-bromoethyl)piperidin-1-yl)-6,7-dimethoxyquinazoline (300 mg, 0.75 mmol) was used instead of 4-(4-(bromomethyl)piperidin-1-yl)-6,7-dimethoxyquinazoline. Yield: 110 mg (34%). MS (ESI, pos. ion) m/z: 428.4 (M+1).

### Step 4: (2-(1-(6,7-dimethoxyquinazolin-4-yl)piperidin-4-yl)ethyl)boronic Acid Formic Acid Salt

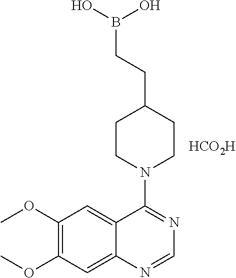

This compound was synthesized by the same method as described at Step 4 in Example 1 except 6,7-dimethoxy-4-(4-(2-(4,4,5,5-tetramethyl-1,3,2-dioxaborolan-2-yl)ethyl)piperidin-1-yl)quinazoline (110 mg, 0.26 mmol) was used instead of 6,7-dimethoxy-4-(4-((4,4,5,5-tetramethyl-1,3,2-dioxaborolan-2-yl)methyl)piperidin-1-yl)quinazoline. Yield: 9.2 mg (10%). MS (ESI, pos. ion) m/z: 346.1 (M+1). ¹H-NMR: (400 MHz, DMSO-d₆, ppm) δ 8.50 (s, 1H), 8.15 (s, 1.5H), 7.42 (brs, 2H), 7.19 (s, 1H), 7.10 (s, 1H), 4.15 (d, J=12.8 Hz, 2H), 3.92-3.89 (m, 6H), 3.06-2.95 (m, 2H), 1.83-1.80 (m, 2H), 1.38-1.29 (m, 5H), 0.65-0.61 (m, 2H).

### Example 3

### Synthesis of (3-(1-(6,7-dimethoxyquinazolin-4-yl)piperidin-4-yl)propyl)boronic Acid

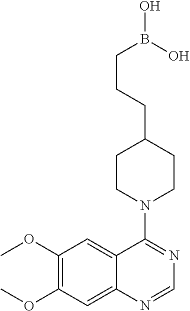 [SMILES: COc1cc2ncnc(N3CCC(CCCB(O)O)CC3)c2cc1OC]

### Step 1: 3-(1-(6,7-dimethoxyquinazolin-4-yl)piperidin-4-yl)propan-1-ol

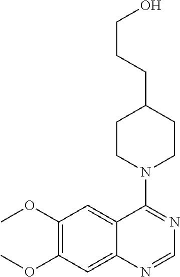 [SMILES: COc1cc2ncnc(N3CCC(CCCO)CC3)c2cc1OC]

The title compound was synthesized by the same method as described at Step 1, in Example 1, except 3-(piperidin-4-yl)propan-1-ol (478.2 mg, 3.339 mmol, 1.50 equiv) was used in place of (piperidin-4-yl)methanol. Yield: 650 mg (88%).

### Step 2: 4-(4-(3-bromopropyl)piperidin-1-yl)-6,7-dimethoxyquinazoline

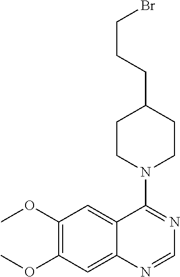 [SMILES: COc1cc2ncnc(N3CCC(CCCBr)CC3)c2cc1OC]

The title compound was synthesized by the same method as described at Step 2, in Example 1, except 3-(1-(6,7-dimethoxyquinazolin-4-yl)piperidin-4-yl)propan-1-ol (650 mg, 1.96 mmol) was used in place of (1-(6,7-dimethoxyquinazolin-4-yl)piperidin-4-yl)methanol. Yield: 450 mg (58%).

### Step 3: 6,7-dimethoxy-4-(4-(3-(4,4,5,5-tetramethyl-1,3,2-dioxaborolan-2-yl)propyl)piperidin-1-yl)quinazoline

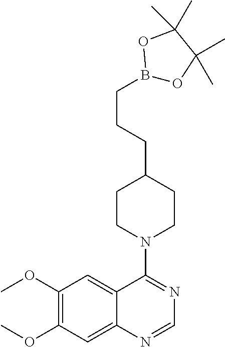

The title compound was synthesized by the same method as described at Step 3 in Example 1 except 4-(4-(3-bromopropyl)piperidin-1-yl)-6,7-dimethoxyquinazoline (300 mg, 0.76 mmol) was used instead of 4-(4-(bromomethyl)piperidin-1-yl)-6,7-dimethoxyquinazoline. Yield: 120 mg (33%).

### Step 4: (3-(1-(6,7-dimethoxyquinazolin-4-yl)piperidin-4-yl)propyl)boronic Acid

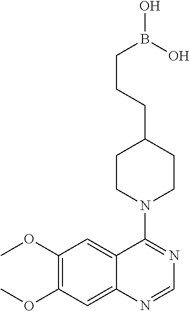 [SMILES: COc1cc2ncnc(N3CCC(CCCB(O)O)CC3)c2cc1OC]

The title compound was synthesized by the same method as described at Step 4, in Example 1, except 6,7-dimethoxy-4-(4-(3-(4,4,5,5-tetramethyl-1,3,2-dioxaborolan-2-yl)propyl)piperidin-1-yl)quinazoline (100 mg, 0.21 mmol) was used instead of 6,7-dimethoxy-4-(4-((4,4,5,5-tetramethyl-1,3,2-dioxaborolan-2-yl)methyl)piperidin-1-yl)quinazoline. Yield: 35.5 mg (47%). MS (ESI, pos. ion) m/z: 360.1 (M+1). ¹H-NMR: (300 MHz, DMSO-d₆, ppm) δ 8.51 (s, 1H), 7.39 (brs, 2H), 7.19 (s, 1H), 7.10 (s, 1H), 4.16-4.12 (m, 2H), 3.92-3.90 (m, 6H), 3.05-2.97 (m, 2H), 1.78-1.82 (m, 2H), 1.54-1.25 (m, 7H), 0.61-0.53 (m, 2H).

### Example 4

### Synthesis of ((1-(6,7-dimethoxyquinazolin-4-yl)piperidin-4-yl)methyl)phosphonic Acid

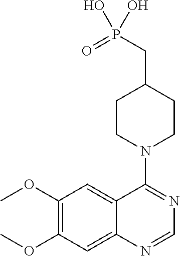 [SMILES: COc1cc2ncnc(N3CCC(CP(=O)(O)O)CC3)c2cc1OC]

### Step 1: diethyl ((1-(6,7-dimethoxyquinazolin-4-yl)piperidin-4-yl)methyl)phosphonate

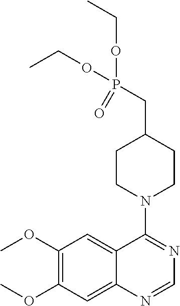 [SMILES: CCOP(=O)(CC1CCN(c2ncnc3cc(OC)c(OC)cc23)CC1)OCC]

A suspension of 4-(4-(bromomethyl)piperidin-1-yl)-6,7-dimethoxyquinazoline (300 mg, 0.819 mmol, 1.00 equiv) in triethyl phosphite (6 mL) was stirred overnight at 140° C. After cooled to room temperature, the reaction mixture was concentrated under reduced pressure. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 10% B to 35% B in 7 min, 220 & 254 nm. The fractions containing the desired product were combined and lyophilized to give 50 mg (11%) of diethyl ((1-(6,7-dimethoxyquinazolin-4-yl)piperidin-4-yl)methyl)phosphonate as a light yellow oil.

### Step 2: ((1-(6,7-dimethoxyquinazolin-4-yl)piperidin-4-yl)methyl)phosphonic Acid

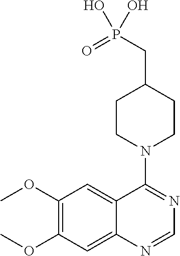 [SMILES: COc1cc2ncnc(N3CCC(CP(=O)(O)O)CC3)c2cc1OC]

To a stirred solution of diethyl ((1-(6,7-dimethoxyquinazolin-4-yl)piperidin-4-yl)methyl)-phosphonate (50 mg, 0.092 mmol, 1.00 equiv, 78%) in DCM (3 mL) was added bromotrimethylsilane (0.5 mL) dropwise. The resulting mixture was stirred overnight at room temperature, quenched with methanol (2 mL) and concentrated under vacuum. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 5% B to 20% B in 7 min, 220 & 254 nm. The fractions containing the desired product were combined and lyophilized to give 11.1 mg (17%) of ((1-(6,7-dimethoxyquinazolin-4-yl)piperidin-4-yl)methyl)phosphonic acid as a white solid. MS (ESI, pos. ion) m/z: 368.0 (M+1). ¹H-NMR (400 MHz, DMSO-d₆, ppm) δ 8.42 (s, 1H), 8.13 (s, 1H), 7.50 (s, 1H), 7.13 (s, 1H), 7.08 (s, 1H), 4.17-4.13 (m, 2H), 3.89-3.85 (m, 6H), 3.12-3.06 (m, 2H), 1.98-1.90 (m, 2H), 1.65-1.50 (m, 3H), 1.40-1.36 (m, 2H).

### Example 5

### Synthesis of (2-(1-(6,7-dimethoxyquinazolin-4-yl)piperidin-4-yl)ethyl)phosphonic Acid Hydrochloride

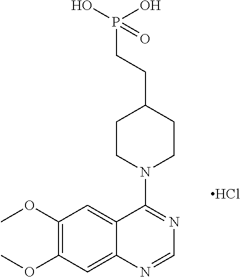 [SMILES: COc1cc2ncnc(N3CCC(CCP(=O)(O)O)CC3)c2cc1OC ; Cl]

### Step 1: diethyl (2-(1-(6,7-dimethoxyquinazolin-4-yl)piperidin-4-yl)ethyl)phosphonate

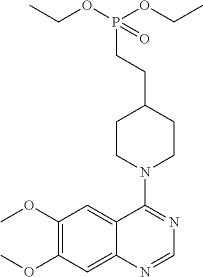 [SMILES: CCOP(=O)(CCC1CCN(c2ncnc3cc(OC)c(OC)cc23)CC1)OCC]

The title compound was synthesized by the same method as described at Step 1, in Example 4, except 4-(4-(2-bromoethyl)piperidin-1-yl)-6,7-dimethoxyquinazoline (150 mg, 0.394 mmol) was used instead of 4-(4-(bromomethyl)piperidin-1-yl)-6,7-dimethoxyquinazoline. Yield: 100 mg (30%). MS (ESI, pos. ion) m/z: 438.1 (M+1).

### Step 2: (2-(1-(6,7-dimethoxyquinazolin-4-yl)piperidin-4-yl)ethyl)phosphonic Acid Hydrochloride

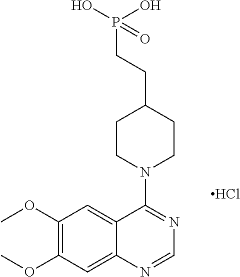 [SMILES: COc1cc2ncnc(N3CCC(CCP(=O)(O)O)CC3)c2cc1OC ; Cl]

The title compound was synthesized by the same method as described at Step 2, in Example 4, except diethyl (2-(1-(6,7-dimethoxyquinazolin-4-yl)piperidin-4-yl)ethyl)phosphonate (150 mg, 0.394 mmol) was used instead of diethyl ((1-(6,7-dimethoxyquinazolin-4-yl)piperidin-4-yl)methyl)-phosphonate. For increasing the water solubility, the crude product was converted to HCl salt by adding 0.5 mL of 1 N hydrochloric acid before purifying by prep-HPLC. Yield: 35.6 mg (24%), MS (ESI, pos. ion) m/z: 382.1 (M+1). ¹H-NMR (300 MHz, DMSO-d₆, ppm) δ 8.50 (s, 1H), 8.14 (s, 1H), 7.35 (s, 1H), 7.19 (s, 1H), 7.10 (s, 1H), 6.51 (s, 1H), 4.16-4.12 (m, 2H), 3.92-3.90 (m, 6H), 3.00-2.91 (m, 2H), 1.84-1.80 (m, 2H), 1.60-1.40 (m, 5H), 1.35-1.24 (m, 2H).

### Example 6

### Synthesis of (3-(1-(6,7-dimethoxyquinazolin-4-yl)piperidin-4-yl)propyl)phosphonic Acid

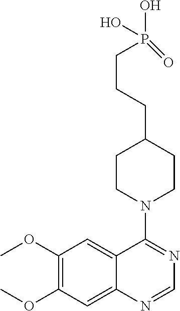 [SMILES: COc1cc2ncnc(N3CCC(CCCP(=O)(O)O)CC3)c2cc1OC]

### Step 1: diethyl 3-(1-(6,7-dimethoxyquinazolin-4-yl)piperidin-4-yl)propylphosphonate

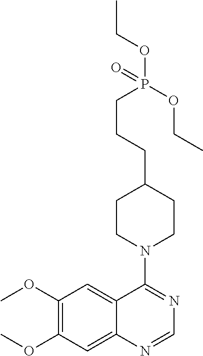 [SMILES: CCOP(=O)(CCCC1CCN(c2ncnc3cc(OC)c(OC)cc23)CC1)OCC]

The compound was synthesized by the same method as described at Step 1, in Example 4. except 4-(4-(3-bromopropyl)piperidin-1-yl)-6,7-dimethoxyquinazoline (200 mg, 0.507 mmol) was used instead of 4-(4-(bromomethyl)piperidin-1-yl)-6,7-dimethoxyquinazoline. Yield: 130 mg (52%).

### Step 2: 3-(1-(6,7-dimethoxyquinazolin-4-yl)piperidin-4-yl)propylphosphonic Acid

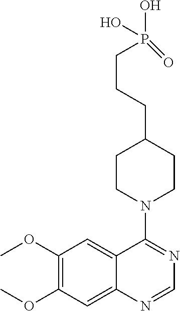 [SMILES: COc1cc2ncnc(N3CCC(CCCP(=O)(O)O)CC3)c2cc1OC]

The title compound was synthesized by the same method as described at Step 2, of Example, except diethyl 3-(1-(6,7-dimethoxyquinazolin-4-yl)piperidin-4-yl)propylphosphonate (130 mg, 0.288 mmol) was used instead of diethyl ((1-(6,7-dimethoxyquinazolin-4-yl)piperidin-4-yl)methyl)-phosphonate. Yield: 57.1 mg (16%). MS (ESI, pos. ion) m/z: 396.3 (M+1). ¹H-NMR (300 MHz, DMSO-d₆, ppm): δ 8.52 (s, 1H), 7.19 (s, 1H), 7.11 (s, 1H), 4.19-4.14 (m, 2H), 3.92-3.90 (m, 6H), 3.08-2.99 (m, 2H), 1.82-1.77 (m, 2H), 1.62-1.38 (m, 5H), 1.35-1.23 (m, 4H). ³¹P-NMR (121 MHz, DMSO-d₆, ppm): δ 26.283 (s, 1P).

### Example 7

### Synthesis of (4-(((6,7-dimethoxyquinazolin-4-yl)amino)methyl)phenyl)boronic Acid Formic Acid Salt

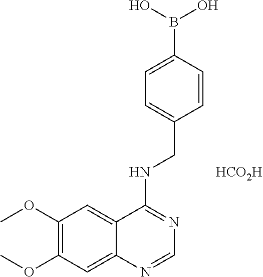 [SMILES: COc1cc2ncnc(NCc3ccc(B(O)O)cc3)c2cc1OC ; "HCO2H"]

To a stirred solution of 4-chloro-6,7-dimethoxyquinazoline (100 mg, 0.445 mmol, 1.00 equiv) in acetonitrile (5.00 mL) were added (4-(aminomethyl)phenyl)boronic acid (101 mg, 0.668 mmol, 1.50 equiv) and potassium carbonate (123 mg, 0.890 mmol, 2.00 equiv). After stirring overnight at 80° C., the reaction mixture was concentrated under vacuum to remove the solvent. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 10% B to 30% B in 8 min, 220 & 254 nm. The fractions containing the desired product were combined and lyophilized to give 80 mg (52%) of (4-(((6,7-dimethoxyquinazolin-4-yl)amino)methyl)phenyl)boronic acid formic acid salt as a white solid. MS (ESI, pos. ion) m/z: 340.3 (M+1). ¹H-NMR (400 MHz, DMSO-d₆, ppm): δ 8.75 (s, 1H), 8.39 (s, 1H), 8.13 (s, 0.69H), 7.97 (s, 2H), 7.75-7.72 (m, 3H), 7.32 (d, J=7.2 Hz, 2H), 7.12 (s, 1H), 4.81 (d, J=4.4 Hz, 2H), 3.91-3.89 (m, 6H).

### Example 8

### Synthesis of (6-(((6,7-dimethoxyquinazolin-4-yl)amino)methyl)pyridin-3-yl)boronic Acid

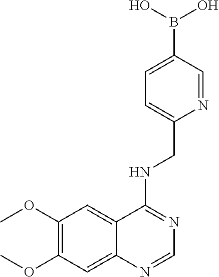 [SMILES: COc1cc2ncnc(NCc3ccc(B(O)O)cn3)c2cc1OC]

To a mixture of 4-chloro-6,7-dimethoxyquinazoline (60 mg, 0.267 mmol, 1.00 equiv) and (6-(aminomethyl)pyridin-3-yl)boronic acid hydrochloride (75 mg, 0.401 mmol, 1.50 equiv) in acetonitrile (2.50 mL) was added DIEA (0.14 mL, 1.080 mmol, 3.00 equiv) at room temperature. The resulting mixture was stirred at 80° C. for 16 h and concentrated under vacuum. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 3% B to 20% B in 7 min, 220 & 254 nm. The fractions containing the desired product were combined and lyophilized to give 8.4 mg (9%) of (6-(((6,7-dimethoxyquinazolin-4-yl)amino)methyl)pyridin-3-yl)boronic acid as an off-white solid. MS (ESI, pos. ion) m/z: 341.1 (M+1). ¹H-NMR (300 MHz, DMSO-d₆, ppm) δ 8.82 (s, 1H), 8.28 (s, 1H), 8.17 (s, 1H), 8.04-8.00 (m, 1H), 7.70 (s, 1H), 7.27 (d, J=7.8 Hz, 1H), 7.11 (s, 1H), 4.84 (s, 2H), 3.90 (s, 6H).

### Example 9

### Synthesis of 6-((6,7-dimethoxyquinazolin-4-yl)amino)-1,3-dihydro-2,1-benzoxaborol-1-ol

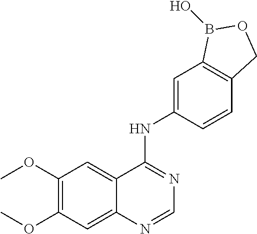 [SMILES: COc1cc2ncnc(Nc3ccc4c(c3)B(O)OC4)c2cc1OC]

To a stirred suspension of 4-chloro-6,7-dimethoxyquinazoline (100 mg, 0.445 mmol, 1.00 equiv) in isopropanol (3.00 mL) was added 6-amino-1,3-dihydro-2,1-benzoxaborol-1-ol hydrochloride (107 mg, 0.579 mmol, 1.30 equiv) at room temperature. After stirred at 85° C. for 2 h, the reaction mixture was cooled to room temperature and filtered. The filter cake was washed with methanol (5 mL) to give 115.8 mg (%) of 6-((6,7-dimethoxyquinazolin-4-yl)amino)-1,3-dihydro-2,1-benzoxaborol-1-ol as a light yellow solid. MS (ESI, pos. ion) m/z: 338.3 (M+1). ¹H-NMR (300 MHz, DMSO-d₆, ppm) δ 11.55 (s, 1H), 9.36 (brs, 1H), 8.80 (s, 1H), 8.36 (s, 1H), 7.96 (s, 1H), 7.75-7.71 (m, 1H), 7.53 (d, J=8.1 Hz, 1H), 7.38 (s, 1H), 5.05 (s, 2H), 4.03-4.00 (m, 6H).

### Example 10

### Synthesis of 5-((6,7-dimethoxyquinazolin-4-yl)amino)-1,3-dihydro-2,1-benzoxaborol-1-ol

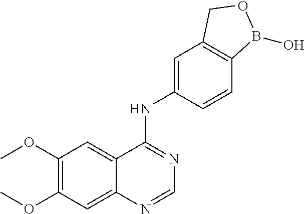 [SMILES: COc1cc2ncnc(Nc3ccc4c(c3)COB4O)c2cc1OC]

The title compound was synthesized by the same method as described in Example 9 except 5-amino-1,3-dihydro-2,1-benzoxaborol-1-ol (99.47 mg, 0.668 mmol, 1.50 equiv) was used instead of 6-amino-1,3-dihydro-2,1-benzoxaborol-1-ol hydrochloride. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 5% B to 15% B in 10 min, 220 & 254 nm. The fractions containing the desired product were combined and lyophilized to give 21.1 mg (14%) of 5-((6,7-dimethoxyquinazolin-4-yl)amino)-1,3-dihydro-2,1-benzoxaborol-1-ol as a light yellow solid. MS (ESI, pos. ion) m/z: 338.3 (M+1). ¹H-NMR: (300 MHz, DMSO-d₆, ppm) δ 9.58 (s, 1H), 8.51 (s, 1H), 8.21 (s, 1H), 8.00 (s, 1H), 7.88 (s, 1H), 7.73-7.71 (m, 2H), 7.21 (s, 1H), 5.02 (s, 2H), 3.97-3.94 (m, 6H).

### Example 11

### Synthesis of 4-(((6,7-dimethoxyquinazolin-4-yl)amino)methyl)-3-fluorophenylboronic Acid Formic Acid Salt

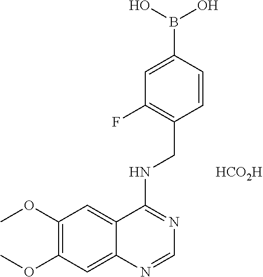 [SMILES: COc1cc2ncnc(NCc3ccc(B(O)O)cc3F)c2cc1OC ; "HCO2H"]

To a stirred mixture of 4-chloro-6,7-dimethoxyquinazoline (150 mg, 0.668 mmol, 1.00 equiv) and 4-(aminomethyl)-3-fluorophenylboronic acid hydrochloride (164 mg, 0.801 mmol, 1.20 equiv) in DMSO (5.00 mL, 70.393 mmol, 105.42 equiv) was added DIEA (0.50 mL, 2.871 mmol, 4.30 equiv) dropwise at room temperature under nitrogen atmosphere. After stirring at 100° C. for 1 h, the reaction mixture was cooled to room temperature and purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 22% B to 42% B in 8 min, 220 & 254 nm. The fractions containing the desired product were combined and lyophilized to give 99.3 mg (36%) of 4-(((6,7-dimethoxyquinazolin-4-yl)amino)methyl)-3-fluorophenylboronic acid formic acid salt as a white solid. MS (ESI, pos. ion) m/z: 358.0 (M+1). ¹H-NMR (400 MHz, DMSO-d₆, ppm) δ 12.66 (brs, 1H), 8.47 (s, 1H), 8.34 (s, 1H), 8.16-8.13 (m, 3H), 7.69 (s, 1H), 7.54-7.51 (m, 2H), 7.33 (t, J=7.6 Hz, 1H), 7.12 (s, 1H), 4.81 (d, J=5.6 Hz, 2H), 3.90-3.87 (m, 6H). ¹⁹F-NMR (376 MHz, DMSO-d₆, ppm) δ −120.60 (s, 1F).

### Example 12

### Synthesis of (3-(((6,7-dimethoxyquinazolin-4-yl)amino)methyl)phenyl)boronic Acid

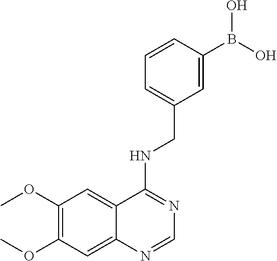 [SMILES: COc1cc2ncnc(NCc3cccc(B(O)O)c3)c2cc1OC]

To a stirred suspension of 4-chloro-6,7-dimethoxyquinazoline (100.00 mg, 0.445 mmol, 1.00 equiv) in isopropanol (4.00 mL) was added (3-(aminomethyl)phenyl)boronic acid hydrochloride (125.15 mg, 0.668 mmol, 1.50 equiv) at room temperature. The resulting mixture was irradiated for 2 h at 120° C. in microwave reactor. After cooled to room temperature, the reaction mixture was concentrated under reduced pressure. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 10% B to 40% B in 7 min, 220 & 254 nm. The fractions containing the desired product were combined and lyophilized to give 10.8 mg (7%) of (3-(((6,7-dimethoxyquinazolin-4-yl)amino)methyl)phenyl)boronic acid as a yellow solid. MS (ESI, pos. ion) m/z: 340.2 (M+1). ¹H-NMR (300 MHz, DMSO-d₆, ppm) δ 8.46-8.44 (m, 1H), 8.33 (s, 1H), 8.25-8.07 (m, 2H), 7.80 (s, 1H), 7.75-7.66 (m, 2H), 7.45-7.38 (m, 1H), 7.31-7.20 (m, 1H), 7.14 (s, 1H), 4.78 (d, J=5.7 Hz, 2H), 3.90-3.85 (m, 6H).

### Example 13

### Synthesis of (4-(((6,7-dimethoxyquinazolin-4-yl)amino)methyl)phenyl)phosphonic Acid

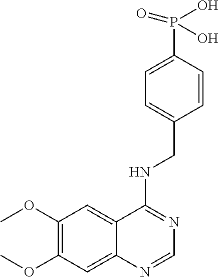 [SMILES: COc1cc2ncnc(NCc3ccc(P(=O)(O)O)cc3)c2cc1OC]

### Step 1: N-((4-bromophenyl)methyl)-6,7-dimethoxyquinazolin-4-amine

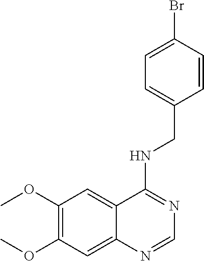 [SMILES: COc1cc2ncnc(NCc3ccc(Br)cc3)c2cc1OC]

To a solution of 4-chloro-6,7-dimethoxyquinazoline (1.0 g, 4.452 mmol, 1.00 equiv) and 1-(4-bromophenyl)methanamine (0.99 g, 5.342 mmol, 1.20 equiv) in acetonitrile (50 mL) was added potassium carbonate (1.85 g, 13.355 mmol, 3.00 equiv). After stirring at 80° C. for 16 h, the reaction mixture was cooled to room temperature, diluted with ethyl acetate (100 mL), washed with water and brine, dried over anhydrous sodium sulfate and filtered. The filtrate was concentrated under reduced pressure. The residue was purified by silica gel column chromatography (petroleum ether/ethyl acetate (1:20)) to afford 1.5 g (81%) of N-((4-bromophenyl)methyl)-6,7-dimethoxyquinazolin-4-amine as an white solid.

### Step 2: diethyl (4-(((6,7-dimethoxyquinazolin-4-yl)amino)methyl)phenyl)phosphonate

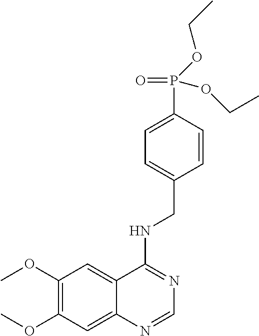 [SMILES: CCOP(=O)(OCC)c1ccc(CNc2ncnc3cc(OC)c(OC)cc23)cc1]

To a stirred solution of N-((4-bromophenyl)methyl)-6,7-dimethoxyquinazolin-4-amine (200 mg, 0.534 mmol, 1.00 equiv) and Pd(AcO)₂ (12.00 mg, 0.053 mmol, 0.10 equiv) in diethyl phosphonate (3 mL) was added triethylamine (541 mg, 5.344 mmol, 10.00 equiv). The resulting mixture was irradiated for 30 min at 120° C. in microwave reactor. After cooling the reaction mixture to room temperature, the reaction mixture was concentrated under reduced pressure. The crude product was purified by prep-TLC (petroleum ether/ethyl acetate (1:1)) to afford 100 mg (33%) of diethyl (4-(((6,7-dimethoxyquinazolin-4-yl)amino)methyl)phenyl)phosphonate as a light yellow oil.

### Step 3: 4-((6,7-dimethoxyquinazolin-4-ylamino)methyl)phenylphosphonic Acid

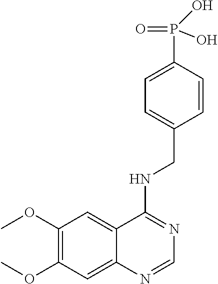 [SMILES: COc1cc2ncnc(NCc3ccc(P(=O)(O)O)cc3)c2cc1OC]

To a stirred solution of diethyl (4-(((6,7-dimethoxyquinazolin-4-yl)amino)methyl)phenyl)-phosphonate (100 mg, 0.178 mmol, 1.00 equiv, 77%) in DCM (3.00 mL) was added bromotrimethylsilane (1 mL) dropwise. After stirring overnight at room temperature, the reaction mixture was quenched with methanol (2 mL) and concentrated under vacuum. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 5% B to 20% B in 7 min, 220 & 254 nm. The fractions containing the desired product were combined and lyophilized to give 14.5 mg (16%) of (4-(((6,7-dimethoxyquinazolin-4-yl)amino)methyl)phenyl)phosphonic acid as a white solid. MS (ESI, pos. ion) m/z: 376.2 (M+1). ¹H-NMR (300 MHz, DMSO-d₆, ppm) δ 8.65 (s, 1H), 8.34 (s, 1H), 7.69-7.58 (m, 3H), 7.44-7.40 (m, 2H), 7.11 (s, 1H), 4.82-4.80 (m, 2H), 3.90-3.88 (m, 6H).

Proceeding analogously as described in Example 13 above, (4-(((7-methoxyquinolin-4-yl)amino)-methyl)phenyl)phosphonic acid was prepared.

### Example 14

### Synthesis of ((4-(6,7-dimethoxyquinazolin-4-yl)phenyl)methyl)phosphonic Acid

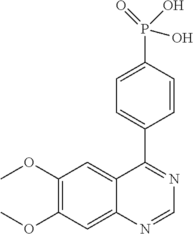 [SMILES: COc1cc2ncnc(-c3ccc(P(=O)(O)O)cc3)c2cc1OC]

### Step 1: (4-(6,7-dimethoxyquinazolin-4-yl)phenyl)methanol

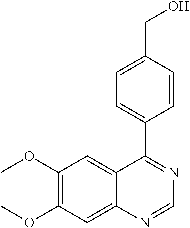 [SMILES: COc1cc2ncnc(-c3ccc(CO)cc3)c2cc1OC]

To a stirred mixture of 4-chloro-6,7-dimethoxyquinazoline (2.00 g, 8.903 mmol, 1.00 equiv), (4-(hydroxymethyl)phenyl)boronic acid (1.35 g, 8.903 mmol, 1.00 equiv) and sodium carbonate (2.83 g, 26.709 mmol, 3.00 equiv) in water (6 mL) and dioxane (24 mL) was added tetrakis(triphenylphosphine)-palladium (1.03 g, 0.890 mmol, 0.10 equiv) under nitrogen atmosphere. The resulting mixture was stirred at 100° C. for 2 h. After cooling the reaction mixture to room temperature, it was diluted with ethyl acetate and filtered through a Celite. The filtrate was washed with water and brine, dried over anhydrous sodium sulfate and filtered. The filtrate was concentrated under reduced pressure. The residue was purified by silica gel column chromatography (petroleum ether/ethyl acetate (4:96)) to afford 1.37 g (50%) of (4-(6,7-dimethoxyquinazolin-4-yl)phenyl)methanol as a light yellow solid.

### Step 2: 4-(4-(bromomethyl)phenyl)-6,7-dimethoxyquinazoline

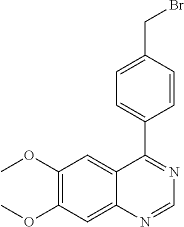 [SMILES: COc1cc2ncnc(-c3ccc(CBr)cc3)c2cc1OC]

To a solution of (4-(6,7-dimethoxyquinazolin-4-yl)phenyl)methanol (1.67 g, 5.636 mmol, 1.00 equiv) and NBS (3.01 g, 16.907 mmol, 3.00 equiv) in DCM (30.00 mL) was added PPh₃ (2.96 g, 11.271 mmol, 2.00 equiv) under nitrogen atmosphere. After stirring at room temperature for 16 h, the reaction mixture was concentrated under vacuum. The residue was purified by prep-TLC (petroleum ether/ethyl acetate (1:1)) to afford 800 mg (34%) of 4-(4-(bromomethyl)phenyl)-6,7-dimethoxyquinazoline as a light yellow solid.

### Step 3: diethyl ((4-(6,7-dimethoxyquinazolin-4-yl)phenyl)methyl)phosphonate

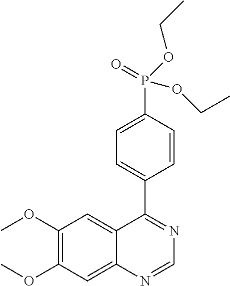 [SMILES: CCOP(=O)(OCC)c1ccc(-c2ncnc3cc(OC)c(OC)cc23)cc1]

A solution of 4-(4-(bromomethyl)phenyl)-6,7-dimethoxyquinazoline (500 mg, 0.585 mmol, 1 equiv, 42% purity) in triethyl phosphite (10 mL, 11.905 mmol, 101.82 equiv) was stirred at 140° C. for 16 h. After cooling the reaction mixture to room temperature, it was concentrated under vacuum. The residue was purified by prep-TLC (petroleum ether/ethyl acetate (1:1)) to afford 40 mg (16%) of diethyl ((4-(6,7-dimethoxyquinazolin-4-yl)phenyl)methyl)phosphonate as an off-white solid. MS (ESI, pos. ion) m/z: 417.3 (M+1).

### Step 4: ((4-(6,7-dimethoxyquinazolin-4-yl)phenyl)methyl)phosphonic Acid

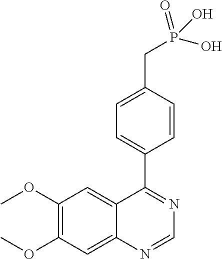 [SMILES: COc1cc2ncnc(-c3ccc(CP(=O)(O)O)cc3)c2cc1OC]

To a solution of diethyl ((4-(6,7-dimethoxyquinazolin-4-yl)phenyl)methyl)phosphonate (40 mg, 0.096 mmol, 1.00 equiv) in DCM (2.00 mL) was added bromotrimethylsilane (147 mg, 0.961 mmol, 10.00 equiv) at 0° C. under nitrogen atmosphere. After stirred at room temperature for 16 h, the reaction mixture was concentrated under vacuum. The crude product was purified by prep-HPLC with the following conditions Column: XBridge Prep C18 OBD Column 30×150 mm, 5 um; Mobile Phase A: Water (0.1% FA), Mobile Phase B: acetonitrile; Flow rate: 60 mL/min; Gradient: 16% B to 30% B in 7 min, 220 & 254 nm. The fractions containing the desired product were combined and lyophilized to give 3.6 mg (10%) of ((4-(6,7-dimethoxyquinazolin-4-yl)phenyl)methyl)phosphonic acid as a light-yellow solid. MS (ESI, pos. ion) m/z: 360.8 (M+1). ¹H-NMR (400 MHz, DMSO-d₆, ppm) δ 9.10 (s, 1H), 7.77-7.71 (m, 2H), 7.50 (d, J=6.8 Hz, 2H), 7.42 (s, 1H), 7.35 (s, 1H), 4.01 (s, 3H), 3.84 (s, 3H), 3.14-3.05 (m, 2H).

### Example 15

### Synthesis of 2-((6,7-dimethoxyquinazolin-4-ylamino)methyl)pyrimidin-5-ylboronic Acid

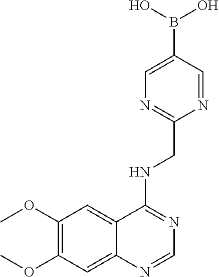 [SMILES: COc1cc2ncnc(NCc3ncc(B(O)O)cn3)c2cc1OC]

### Step 1: (2-(aminomethyl)pyrimidin-5-yl)boronic Acid Hydrochloride

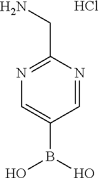 [SMILES: Cl ; NCc1ncc(B(O)O)cn1]

To a solution of 5-(4,4,5,5-tetramethyl-1,3,2-dioxaborolan-2-yl)pyrimidine-2-carbonitrile in MeOH (10 mL) was added Pd/C (10%, 40 mg) and 0.5 mL concentrated hydrochloric acid under nitrogen atmosphere. The resulting mixture was stirred overnight at room temperature under hydrogen atmosphere using a hydrogen balloon. The reaction mixture was filtered through a Celite pad. The filtrate was concentrated under reduced pressure to give 200 mg (crude) of (2-(aminomethyl)pyrimidin-5-yl)boronic acid hydrochloride, which was used for the next step directly without further purification.

### Step 2: 2-((6,7-dimethoxyquinazolin-4-ylamino)methyl)pyrimidin-5-ylboronic Acid

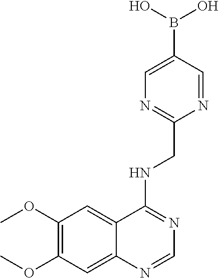 [SMILES: COc1cc2ncnc(NCc3ncc(B(O)O)cn3)c2cc1OC]

To a stirred suspension of (2-(aminomethyl)pyrimidin-5-yl)boronic acid hydrochloride (crude) (200 mg, 1.056 mmol, 1 equiv) in isopropanol (6 mL) was added 4-chloro-6,7-dimethoxyquinazoline (237 mg, 1.056 mmol, 1.00 equiv) at room temperature. The resulting mixture was stirred at 80° C. for 2 h. After cooling the reaction mixture to room temperature, it was concentrated under vacuum. The crude product was purified by prep-HPLC with the following conditions Column: XBridge Shield RP18 OBD Column 19×150 mm, 5 um; Mobile Phase A: Water (0.1% FA), Mobile Phase B: acetonitrile; Flow rate: 20 mL/min; Gradient: 5% B to 15% B in 8 min, 220 & 254 nm. The fractions containing the desired product were combined and lyophilized to give 61.7 mg (16%) of 2-((6,7-dimethoxyquinazolin-4-ylamino)methyl)pyrimidin-5-ylboronic acid as an off-white solid. MS (ESI, pos. ion) m/z: 342.3 (M+1). ¹H-NMR (400 MHz, D₂O/DMSO-d₆, ppm) δ 8.93 (s, 2H), 8.24 (s, 1H), 8.19 (s, 1H), 7.69 (s, 1H), 7.13 (s, 1H), 4.96 (s, 2H), 3.93 (s, 6H).

### Example 16

### Synthesis of 4-((6,7-dimethoxyquinazolin-4-yl)amino)phenyl Sulfamate

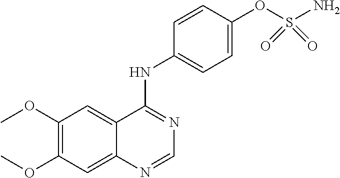 [SMILES: COc1cc2ncnc(Nc3ccc(OS(N)(=O)=O)cc3)c2cc1OC]

### Step 1: 4-((6,7-dimethoxyquinazolin-4-yl)amino)phenol

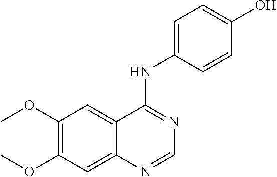 [SMILES: COc1cc2ncnc(Nc3ccc(O)cc3)c2cc1OC]

A mixture of 4-chloro-6,7-dimethoxyquinazoline (110 mg, 0.490 mmol, 1.00 equiv) and aminophenol (80 mg, 0.735 mmol, 1.50 equiv) in isopropanol (5 mL, 65.421 mmol, 133.60 equiv) was stirred at 85° C. for 2 h. After cooling the reaction mixture to room temperature, it was filtered and the filter cake was washed with MeOH (10 mL×2). The filtrate was concentrated under reduced pressure to afford 130 mg (89%) of 4-((6,7-dimethoxyquinazolin-4-yl)amino)phenol as an off-white solid.

### Step 2: 4-((6,7-dimethoxyquinazolin-4-yl)amino)phenyl Sulfamate

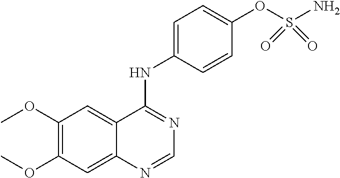 [SMILES: COc1cc2ncnc(Nc3ccc(OS(N)(=O)=O)cc3)c2cc1OC]

To a stirred mixture of 4-((6,7-dimethoxyquinazolin-4-yl)amino)phenol (110 mg, 0.370 mmol, 1.00 equiv) in N,N-dimethylacetamide (4 mL, 43.021 mmol, 116.28 equiv) was added sulfamoyl chloride (86 mg, 0.740 mmol, 2.00 equiv) at room temperature under nitrogen atmosphere. After stirring at room temperature for 1 h, the reaction mixture was filtered to give 3.5 mL of a clear solution, which was purified by prep-HPLC with the following conditions Column: XBridge Shield RP18 OBD Column 19×150 mm, 5 um; Mobile Phase A: Water (10 mmol/L NH₄HCO₃), Mobile Phase B: acetonitrile; Flow rate: 20 mL/min; Gradient: 28% B to 38% B in 7 min, 220 & 254 nm. The fractions containing the desired product were combined and lyophilized to give 85 mg (61%) of 4-((6,7-dimethoxyquinazolin-4-yl)amino)phenyl sulfamate as an off-white solid. MS (ESI, pos. ion) m/z: 377.0 (M+1). ¹H-NMR: (400 MHz, DMSO-d₆, ppm) δ 9.57 (s, 1H), 8.46 (s, 1H), 8.00 (s, 2H), 7.85-7.83 (m, 3H), 7.31-7.28 (m, 2H), 7.20 (s, 1H), 3.97-3.93 (m, 6H).

### Example 17

### Synthesis of 4-(6,7-dimethoxyquinazolin-4-yl)benzyl Sulfamate

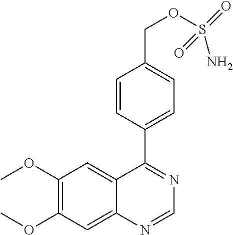 [SMILES: COc1cc2ncnc(-c3ccc(COS(N)(=O)=O)cc3)c2cc1OC]

### Step 1: (4-(6,7-dimethoxyquinazolin-4-yl)phenyl)methanol

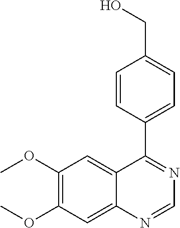 [SMILES: COc1cc2ncnc(-c3ccc(CO)cc3)c2cc1OC]

To a stirred mixture of 4-chloro-6,7-dimethoxyquinazoline (0.30 g, 1.335 mmol, 1.00 equiv) and (4-(hydroxymethyl)phenyl)boronic acid (0.20 g, 1.335 mmol, 1.00 equiv) in water (6 mL) and dioxane (24 mL) was added tetrakis(triphenylphosphine)palladium (0.15 g, 0.134 mmol, 0.10 equiv) under nitrogen atmosphere. The resulting mixture was purged with nitrogen for 5 minutes and stirred at 100° C. for 2 h. After cooled to room temperature, the reaction mixture was diluted with ethyl acetate (100 mL) and filtered through a Celite. The filtrate was washed with water (100 mL×3) and brine (100 mL×2), dried over anhydrous sodium sulfate and filtered. The filtrate was concentrated under reduced pressure. The residue was purified by silica gel column chromatography (petroleum ether/ethyl acetate (4:96)) to afford 300 mg (44%) of (4-(6,7-dimethoxyquinazolin-4-yl)phenyl)methanol as a light yellow solid.

### Step 2: 4-(6,7-dimethoxyquinazolin-4-yl)benzyl Sulfamate

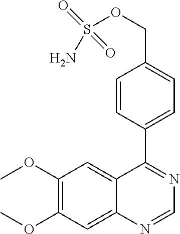 [SMILES: COc1cc2ncnc(-c3ccc(COS(N)(=O)=O)cc3)c2cc1OC]

The title compound was synthesized by the same method as described at Step 2, Example 16, except (4-(6,7-dimethoxyquinazolin-4-yl)phenyl)methanol (120 mg, 0.40 mmol) was used instead of 4-((6,7-dimethoxyquinazolin-4-yl)amino)phenol. Yield: 70.7 mg (46%). MS (ESI, pos. ion) m/z: 376.2 (M+1). ¹H-NMR (DMSO-d₆, ppm): δ 9.15 (s, 1H), 7.90 (d, J=8.1 Hz, 2H), 7.73 (s, 2H), 7.66 (d, J=8.1 Hz, 2H), 7.45 (s, 1H), 7.32 (s, 1H), 5.22 (s, 2H), 4.02 (s, 3H), 3.85 (s, 3H).

Proceeding analogously as described in Example 17 above, ((4-((6,7-dimethoxyquinazolin-4-yl)amino)phenoxy)methyl)boronic acid was prepared

### Example 18

### Synthesis of 4-(((6,7-dimethoxyquinazolin-4-yl)amino)methyl)piperidine-1-sulfonamide

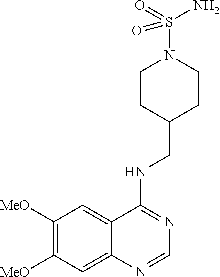 [SMILES: COc1cc2ncnc(NCC3CCN(S(N)(=O)=O)CC3)c2cc1OC]

### Step 1: tert-butyl N-((1-((((tert-butoxy)carbonyl)amino)sulfonyl)piperidin-4-yl)methyl)carbamate

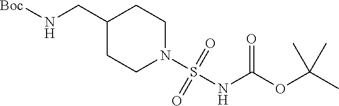 [SMILES: CC(C)(C)OC(=O)NCC1CCN(S(=O)(=O)NC(=O)OC(C)(C)C)CC1]

To a stirred solution of tert-butyl N-((piperidin-4-yl)methyl)carbamate (760 mg, 3.546 mmol, 1 equiv) and 1-((((tert-butoxy)carbonyl)azanediyl)sulfonyl)-4-(dimethylamino)pyridin-1-ium (1068.71 mg, 3.546 mmol, 1 equiv) in DCM (10 mL) was added DIEA (916.67 mg, 7.093 mmol, 2 equiv) at room temperature. After stirring for 2 h, the resulting mixture was filtered, and the filter cake was washed with dichloromethane (10 mL). The filtrate was washed with water, brine, dried over anhydrous sodium sulfate and filtered. The filtrate was concentrated under reduced pressure to afford 900 mg of tert-butyl N-((1-((((tert-butoxy)carbonyl)amino)sulfonyl)piperidin-4-yl)methyl)carbamate.

### Step 2: 4-(aminomethyl)piperidine-1-sulfonamide Hydrochloride

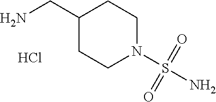 [SMILES: NCC1CCN(S(N)(=O)=O)CC1 ; Cl]

To a stirred solution of tert-butyl N-((1-((((tert-butoxy)carbonyl)amino)sulfonyl)piperidin-4-yl)methyl)carbamate (900 mg, 2.287 mmol, 1 equiv) in 1,4-dioxane (5 mL) was added 4 M HCl (gas) in 1,4-dioxane (10 mL) at room temperature. After stirring for 1 h at 50° C., the resulting mixture was allowed to cool down to room temperature, and diluted with diethyl ether. The mixture was concentrated under reduced pressure to afford 600 mg of 4-(aminomethyl)piperidine-1-sulfonamide hydrochloride.

### Step 3: 4-(((6,7-dimethoxyquinazolin-4-yl)amino)methyl)piperidine-1-sulfonamide

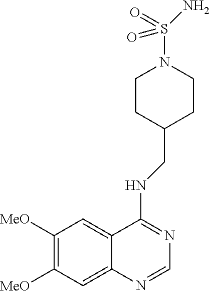 [SMILES: COc1cc2ncnc(NCC3CCN(S(N)(=O)=O)CC3)c2cc1OC]

To a stirred solution of 4-(aminomethyl)piperidine-1-sulfonamide hydrochloride (400 mg, 1.741 mmol, 1 equiv) and 4-chloro-6,7-dimethoxyquinazoline (391.15 mg, 1.741 mmol, 1 equiv) in DMF (5 mL) was added K₂CO₃ (721.95 mg, 5.224 mmol, 3 equiv) at room temperature. After stirring overnight, the reaction mixture was filtered. The filtrate was concentrated under reduced pressure. The residue was purified by reversed-phase flash chromatography with the following conditions: Column: SunFire C18 OBD Prep Column, 19×250 mm, 5 um; Mobile Phase A: Water (0.1% FA), Mobile Phase B: ACN; Flow rate: 20 mL/min; Gradient: 15% B to 40% B in 11 min; 254/220 nm; Rt: 9.12 min to afford 49 mg of 4-(((6,7-dimethoxyquinazolin-4-yl)amino)methyl)piperidine-1-sulfonamide. LC-MS-PH-RIS-005-2-0: (ES, m/z): 382 (M+H)⁺; ¹H-NMR-PH-RIS-005-2-0: (DMSO-d6, ppm): 8.31 (s, 1H), 7.94 (t, 1H), 7.59 (s, 1H), 7.06 (s, 1H), 6.66 (s, 2H), 3.87 (s, 6H), 3.47-3.40 (m, 4H), 2.48-2.42 (m, 2H), 1.82-1.78 (m, 3H), 1.31-1.24 (m, 2H).

### Example 19

### Synthesis of (4-(((6,7-dimethoxyquinazolin-4-yl)oxy)methyl)phenyl)boronic Acid

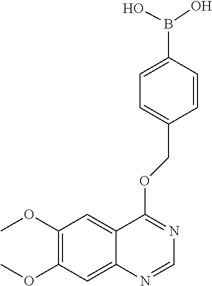 [SMILES: COc1cc2ncnc(OCc3ccc(B(O)O)cc3)c2cc1OC]

To a stirred solution of 4-chloro-6,7-dimethoxyquinazoline (100 mg, 0.445 mmol, 1.00 equiv) and 4-(hydroxymethyl)phenylboronic acid (101 mg, 0.668 mmol, 1.50 equiv) in DMF (5.00 mL) was added cesium carbonate (363 mg, 1.113 mmol, 2.5 equiv) at room temperature. The resulting mixture was irradiated for 2 h at 120° C. in a microwave reactor. After cooling the reaction mixture to room temperature, it was filtered and the filter cake was washed with EA. The filtrate was concentrated under reduced pressure. The crude product was purified by prep-HPLC under the following conditions Column: XBridge Shield RP18 OBD Column 19×150 mm, 5 um; Mobile Phase A: Water (0.1% FA), Mobile Phase B: acetonitrile; Flow rate: 20 mL/min; Gradient: 20% B to 40% B in 7 min, 220 & 254 nm. The fractions containing the desired product were combined and lyophilized to give 35.5 mg (15%) of (4-(((6,7-dimethoxyquinazolin-4-yl)oxy)methyl)phenyl)boronic acid as a white solid. MS (ESI, pos. ion) m/z: 341.0 (M+1). ¹H-NMR (DMSO-d₆, ppm): 8.65 (s, 1H), 8.15 (s, 2H), 7.83-7.81 (d, 2H), 7.51-7.49 (d, 2H), 7.37-7.33 (d, 2H), 5.65 (s, 2H), 3.96-3.91 (d, 6H).

### Example 20

### Synthesis of 6-(((6,7-dimethoxyquinazolin-4-yl)oxy)methyl)-3H-2,1-benzoxaborol-1-ol

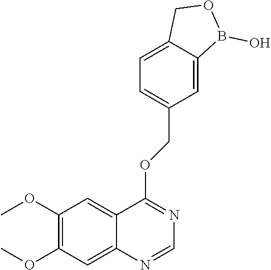 [SMILES: COc1cc2ncnc(OCc3ccc4c(c3)B(O)OC4)c2cc1OC]

### Step 1: (3-bromo-4-(hydroxymethyl)phenyl)methano

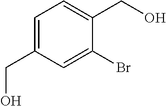 [SMILES: OCc1ccc(CO)c(Br)c1]

To a solution of 1,4-dimethyl 2-bromobenzene-1,4-dicarboxylate (2.00 g, 7.324 mmol, 1.00 equiv) in THF (30.00 mL) was added LiBH₄ (478.63 mg, 21.972 mmol, 3.00 equiv) slowly at 0° C. MeOH (5 mL) was added to the mixture. After stirring overnight at room temperature, the reaction mixture was quenched with 1 N HCl to pH=6-7. The resulting mixture was extracted with ethyl acetate and the combined organic layers were dried over anhydrous sodium sulfate and filtered. The filtrate was concentrated under reduced pressure to afford 1.2 g (76%) of (3-bromo-4-(hydroxymethyl)phenyl)methanol as an off-white solid.

### Step 2: 2-((3-bromo-4-((oxan-2-yloxy)methyl)phenyl)methoxy)oxane

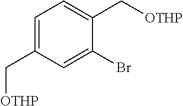

To a solution of (3-bromo-4-(hydroxymethyl)phenyl)methanol (1.20 g, 5.528 mmol, 1.00 equiv) and p-toluenesulfonic acid (47.6 mg, 0.276 mmol, 0.05 equiv) in DMF (20 mL) was added DHP (1.86 g, 22.112 mmol, 4.00 equiv). After stirring overnight at room temperature, the reaction mixture was quenched with saturated NaHCO₃ solution and extracted with EA. The combined organic layers were washed with brine, dried over anhydrous sodium sulfate and filtered. The filtrate was concentrated under reduced pressure. The residue was purified by silica gel column chromatography (MeOH/DCM (1:10)) to afford 1.2 g (56%) of 2-((3-bromo-4-((oxan-2-yloxy)methyl)phenyl)-methoxy)oxane as a colorless oil.

### Step 3: 6-(hydroxymethyl)-1,3-dihydro-2,1-benzoxaborol-1-ol

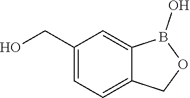 [SMILES: OCc1ccc2c(c1)B(O)OC2]

To a stirred solution of 2-((3-bromo-4-((oxan-2-yloxy)methyl)phenyl)methoxy)oxane (1.20 g, 3.114 mmol, 1.00 equiv) in THF (20.00 mL) was added n-BuLi in hexanes (0.44 mL, 6.870 mmol, 1.50 equiv) dropwise at −78° C. under argon atmosphere. After stirring at −78° C. for 30 min, B(Oi-Pr)₃ (0.88 g, 4.672 mmol, 1.50 equiv) was added. The reaction mixture was allowed to warm to room temperature and stirred overnight. Then it was quenched with 6 N HCl (15 mL) and stirred at room temperature for 6 h. The resulting mixture was extracted with EA and the combined organic layers were washed with brine, dried over anhydrous sodium sulfate and filtered. The filtrate was concentrated under reduced pressure. The crude product (120 mg) was purified by CombiFlash with the following conditions Column: C18, 120 g; Mobile Phase A: Water, Mobile Phase B: acetonitrile; Flow rate: 60 mL/min; Gradient: 10% B to 30% B in 10 min, 220 & 254 nm. The fractions containing the desired product were combined and lyophilized to give 300 mg (59%) of 6-(hydroxymethyl)-1,3-dihydro-2,1-benzoxaborol-1-ol as a white solid.

### Step 4: 6-(((6,7-dimethoxyquinazolin-4-yl)oxy)methyl)-3H-2,1-benzoxaborol-1-ol

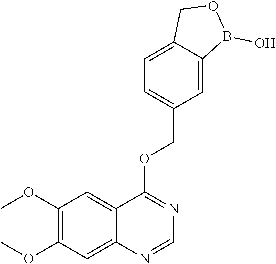 [SMILES: COc1cc2ncnc(OCc3ccc4c(c3)B(O)OC4)c2cc1OC]

A mixture of 4-chloro-6,7-dimethoxyquinazoline (100 mg, 0.445 mmol, 1.00 equiv), 6-(hydroxymethyl)-1,3-dihydro-2,1-benzoxaborol-1-ol (109 mg, 0.668 mmol, 1.50 equiv) and NaOH (35.6 mg, 0.890 mmol, 2.00 equiv) in DMF (4 mL, 0.055 mmol) was stirred at 80° C. for 1 h. After cooling the reaction mixture to room temperature, it was concentrated and purified by prep-HPLC under the following conditions Column: XBridge Shield RP18 OBD Column 19×150 mm, 5 um; Mobile Phase A: Water (0.1% FA), Mobile Phase B: acetonitrile; Flow rate: 20 mL/min; Gradient: 22% B to 32% B in 10 min, 220 & 254 nm. The fractions containing the desired product were combined and lyophilized to give 37.4 mg (23%) of 6-(((6,7-dimethoxyquinazolin-4-yl)oxy)methyl)-3H-2,1-benzoxaborol-1-ol as a white solid. MS (ESI, pos. ion) m/z: 353.0 (M+1). ¹H-NMR (300 MHz, DMSO-d₆, ppm) δ 9.22 (s, 1H), 8.66 (s, 1H), 7.88 (s, 1H), 7.68-7.65 (m, 1H), 7.46 (d, J=7.5 Hz, 1H), 7.34 (d, J=4.8 Hz, 2H), 5.70 (s, 2H), 5.01 (s, 2H), 3.96-3.91 (m, 6H).

### Example 21

### Synthesis of 5-(((6,7-dimethoxyquinazolin-4-yl)oxy)methyl)-3H-2,1-benzoxaborol-1-ol

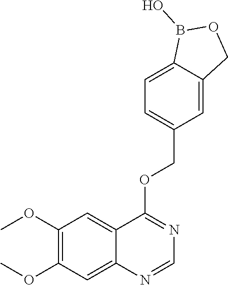 [SMILES: COc1cc2ncnc(OCc3ccc4c(c3)COB4O)c2cc1OC]

### Step 1: (4-bromo-3-(hydroxymethyl)phenyl)methanol

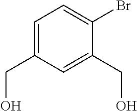 [SMILES: OCc1ccc(Br)c(CO)c1]

To a stirred solution of LiBH₄ (1.07 g, 48.974 mmol, 3 equiv) in THF (25 mL) was added TMSCl (5.32 g, 48.974 mmol, 3.00 equiv) dropwise at 0° C. under argon atmosphere. After stirring 30 min at room temperature, a solution of 4-bromobenzene-1,3-dicarboxylic acid (4.00 g, 16.325 mmol, 1.00 equiv) in THF (15 mL) was added. After stirring overnight, the reaction mixture was quenched with MeOH (25 mL) in an ice bath and extracted with EA. The combined organic layers were washed with brine, dried over anhydrous sodium sulfate and filtered. The filtrate was concentrated under reduced pressure to afford 1.5 g (40%) of (4-bromo-3-(hydroxymethyl)phenyl)methanol as a white solid.

### Step 2: 2-((2-bromo-5-((oxan-2-yloxy)methyl)phenyl)methoxy)oxane

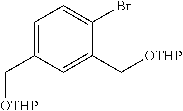

The title compound was synthesized by the same method as described at Step 2 in Example 20except (4-bromo-3-(hydroxymethyl)phenyl)methanol (1.50 g, 6.91 mmol) was used. Yield: 2.00 g (70%).

### Step 3: 5-(hydroxymethyl)-3H-2,1-benzoxaborol-1-ol

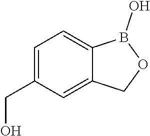 [SMILES: OCc1ccc2c(c1)COB2O]

The title compound was synthesized by the same method as described at Step 3, Example 20, except 2-((2-bromo-5-((oxan-2-yloxy)methyl)phenyl)methoxy)oxane (1.50 g, 6.91 mmol) was used. Yield: 0.6 g (93%).

### Step 4: 5-(((6,7-dimethoxyquinazolin-4-yl)oxy)methyl)-3H-2,1-benzoxaborol-1-ol

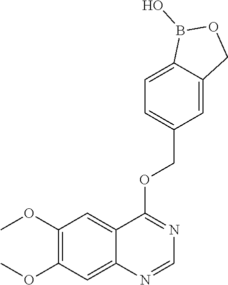 [SMILES: COc1cc2ncnc(OCc3ccc4c(c3)COB4O)c2cc1OC]

The title compound was synthesized by the same method as described at Step 4, Example 20 except 5-(hydroxymethyl)-3H-2,1-benzoxaborol-1-ol (108 mg, 0.668 mmol, 1.50 equiv) was used. Yield: 25.5 mg (16%). MS (ESI, pos. ion) m/z: 353.2 (M+1). ¹H-NMR (300 MHz, DMSO-d₆, ppm) δ 9.21 (s, 1H), 8.65 (s, 1H), 7.76 (d, J=7.5 Hz, 1H), 7.56-7.50 (m, 2H), 7.38 (s, 1H), 7.34 (s, 1H), 5.72 (s, 2H), 5.01 (s, 2H), 3.97-3.91 (m, 6H).

### Example 22

### Synthesis of (4-(((8-methoxyquinolin-4-yl)amino)methyl)phenyl)boronic Acid

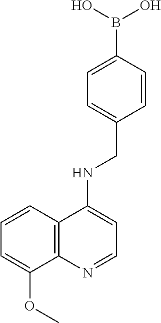 [SMILES: COc1cccc2c(NCc3ccc(B(O)O)cc3)ccnc12]

To a stirred suspension of 4-chloro-8-methoxyquinoline (100 mg, 0.516 mmol, 1.00 equiv) in isopropanol (3.00 mL) was added (4-(aminomethyl)phenyl)boronic acid hydrochloride (116 mg, 0.620 mmol, 1.20 equiv) at room temperature. The resulting mixture was irradiated for 2 h at 120° C. in a microwave reactor. After cooling the reacting mixture to room temperature, it was concentrated under reduced pressure. The crude product was purified by prep-HPLC with the following conditions Column: XBridge Prep C18 OBD Column 19×150 mm, 5 um; Mobile Phase A: Water (10 mmol/L NH₄HCO₃), Mobile Phase B: acetonitrile; Flow rate: 20 mL/min; Gradient: 15% B to 40% B in 7 min, 220 & 254 nm. The fractions containing the desired product were combined and lyophilized to give 31.9 mg (19%) of (4-(((8-methoxyquinolin-4-yl)amino)methyl)phenyl)boronic acid as a white solid. MS (ESI, pos. ion) m/z: 309.3 (M+1). ¹H-NMR (300 MHz, DMSO-d₆, ppm) δ 8.24 (d, J=5.4 Hz, 1H), 7.98-7.81 (m, 4H), 7.73 (d, J=7.8 Hz, 2H), 7.42-7.31 (m, 3H), 7.11 (d, J=7.2 Hz, 1H), 6.35 (d, J=5.4 Hz, 1H), 4.56 (d, J=5.7 Hz, 2H), 3.91 (s, 3H).

### Example 23

### Synthesis of (4-(((8-hydroxyquinolin-4-yl)amino)methyl)phenyl)boronic Acid

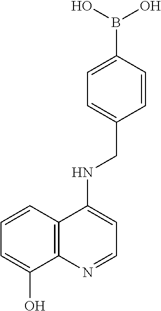 [SMILES: OB(O)c1ccc(CNc2ccnc3c(O)cccc23)cc1]

To a stirred suspension of (4-(((8-methoxyquinolin-4-yl)amino)methyl)phenyl)boronic acid (100 mg, 0.292 mmol, 1.00 equiv, 90%) in DCM (5 mL) was added BBr₃ (732 mg, 2.921 mmol, 10.00 equiv) dropwise at room temperature. After stirring overnight, the reaction mixture was concentrated under reduced pressure. The crude product was purified by prep-HPLC with the following conditions Column: XBridge Prep C18 OBD Column 19×150 mm, 5 um; Mobile Phase A: Water (0.1% FA), Mobile Phase B: acetonitrile; Flow rate: 20 mL/min; Gradient: 5% B to 22% B in 8 min, 220 & 254 nm. The fractions containing the desired product were combined and lyophilized to give 12.1 mg (14%) of (4-(((8-hydroxyquinolin-4-yl)amino)methyl)phenyl)boronic acid as an off-white solid. MS (ESI, pos. ion) m/z: 295.2 (M+1). ¹H-NMR (300 MHz, DMSO-d₆, ppm) δ 9.10-8.85 (m, 1H), 8.24 (s, 1H), 7.74-7.64 (m, 1H), 7.49-6.82 (m, 9H), 6.26-6.00 (m, 1H), 4.55-4.32 (m, 2H).

### Example 24

### Synthesis of 4-(((6,7-dimethoxyquinazolin-4-yl)amino)methyl)-3,5-difluorophenylboronic Acid Hydrochloride

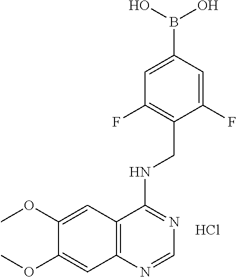 [SMILES: COc1cc2ncnc(NCc3c(F)cc(B(O)O)cc3F)c2cc1OC ; Cl]

### Step 1: (E)-N-((4-bromo-2,6-difluorophenyl)methylidene)hydroxylamine

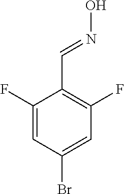 [SMILES: O/N=C/c1c(F)cc(Br)cc1F]

To a stirred solution of 4-bromo-2,6-difluorobenzaldehyde (1.00 g, 4.525 mmol, 1.00 equiv) in ethanol (20 mL) was added a solution of hydroxylamine hydrochloride (629 mg, 9.050 mmol, 2.00 equiv) in water (10 mL) and a solution of sodium carbonate (480 mg, 4.525 mmol, 1.00 equiv) in water (10 mL) dropwise at room temperature under nitrogen atmosphere. After stirring at room temperature for 1 h, the reaction mixture was concentrated under reduced pressure. The residue was treated with ethanol (10 mL). The solid was collected by filtration, washed with ethanol (5 mL) and dried under vacuum to give 950 mg (87%) of (E)-N-((4-bromo-2,6-difluorophenyl)methylidene)hydroxylamine as a white solid.

### Step 2: (E)-N-((2,6-difluoro-4-(4,4,5,5-tetramethyl-1,3,2-dioxaborolan-2-yl)phenyl)-methylidene) hydroxylamine

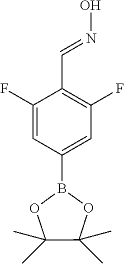 [SMILES: CC1(C)OB(c2cc(F)c(/C=N/O)c(F)c2)OC1(C)C]

To a stirred solution of (E)-N-((4-bromo-2,6-difluorophenyl)methylidene)hydroxylamine (800 mg, 3.390 mmol, 1.00 equiv) in 1,4-dioxane (20 mL) were added bis(pinacolato)diboron (2.15 g, 8.474 mmol, 2.50 equiv), potassium acetate (832 mg, 8.474 mmol, 2.50 equiv) and (1,1′-bis(diphenylphosphino)ferrocene)dichloropalladium(II) (248 mg, 0.339 mmol, 0.10 equiv) at room temperature under argon atmosphere. The resulting mixture was stirred at 90° C. for 6.5 h. After cooled to room temperature, the reaction mixture was filtered and the filtrate was concentrated under reduced pressure. The residue was purified by silica gel column chromatography (dichloromethane/methanol (10:1)) to afford 870 mg (81%) of (E)-N-((2,6-difluoro-4-(4,4,5,5-tetramethyl-1,3,2-dioxaborolan-2-yl)phenyl)methylidene)hydroxylamine as a brown solid.

### Step 3: 1-(2,6-difluoro-4-(4,4,5,5-tetramethyl-1,3,2-dioxaborolan-2-yl)phenyl)methanamine Hydrochloride

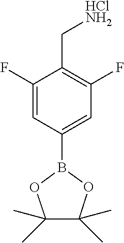

To a stirred solution of (E)-N-((2,6-difluoro-4-(4,4,5,5-tetramethyl-1,3,2-dioxaborolan-2-yl)phenyl)methylidene) hydroxylamine (950 mg, 3.020 mmol, 1.00 equiv) in ethanol (15 mL) were added 10% palladium carbon (95 mg) and concentrated hydrochloric acid (3 mL) dropwise at room temperature. The resulting mixture was vigorously stirred for 3 h at ambient temperature under a hydrogen atmosphere (2-3 atm.) and then filtered through Celite. The filtrate was concentrated under reduced pressure. The residue was treated with ethyl acetate/diethyl ether (1:2, 15 mL). The solid was collected by filtration, washed with diethyl ether (10 mL) and dried in vacuo to afford 590 mg (61%) of 1-(2,6-difluoro-4-(4,4,5,5-tetramethyl-1,3,2-dioxaborolan-2-yl)phenyl)methanamine hydrochloride as an off-white solid.

### Step 4: 4-(((6,7-dimethoxyquinazolin-4-yl)amino)methyl)-3,5-difluorophenylboronic Acid Hydrochloride

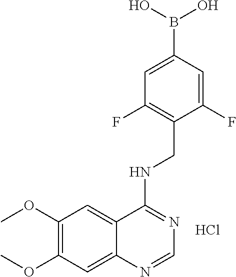 [SMILES: COc1cc2ncnc(NCc3c(F)cc(B(O)O)cc3F)c2cc1OC ; Cl]

To a stirred solution of 4-chloro-6,7-dimethoxyquinazoline (125 mg, 0.556 mmol, 1.00 equiv) and 1-(2,6-difluoro-4-(4,4,5,5-tetramethyl-1,3,2-dioxaborolan-2-yl)phenyl)methanamine hydrochloride (204 mg, 0.668 mmol, 1.20 equiv) in dimethyl sulfoxide (4 mL) was added N,N-diisopropylethylamine (0.4 mL) dropwise at room temperature under nitrogen atmosphere. The resulting mixture was stirred at 100° C. for 1 h under nitrogen atmosphere. After cooled to room temperature, the reaction mixture was filtered. The filtrate was purified by CombiFlash with the following conditions: Column: C18, 120 g; Mobile Phase A: water, Mobile Phase B: acetonitrile; Flow rate: 50 mL/min; Gradient: 5% B to 50% B in 35 min; 220 & 254 nm. The fractions containing the desired product were combined and concentrated under reduced pressure to give the crude product, which was added several drops of 1N hydrochloric acid to improve the solubility and further purified by prep-HPLC with the following conditions: Column: XBridge Prep C18 OBD Column 19×150 mm, 5 um; Mobile Phase A: Water (0.1% FA), Mobile Phase B: acetonitrile; Flow rate: 20 mL/min; Gradient: 10% B to 25% B in 7 min, 220 & 254 nm. The fractions containing the desired product were combined and lyophilized to give 78.1 mg (33%) of 4-(((6,7-dimethoxyquinazolin-4-yl)amino)methyl)-3,5-difluorophenylboronic acid hydrochloride as a white solid. MS (ESI, pos. ion) m/z: 376.0 (M+1). ¹H NMR: (400 MHz, DMSO-d₆, ppm) δ 12.70 (brs, 1H), 8.42-8.38 (m, 2H), 8.28-8.14 (m, 2H), 7.64 (s, 1H), 7.43-7.39 (m, 2H), 7.10 (s, 1H), 4.80 (s, 2H), 3.89-3.84 (m, 6H). ¹⁹F-NMR: (376 MHz, DMSO-d₆, ppm) δ −115.54 (s, 2F).

### Example 25

### Synthesis of 4-(6,7-dimethoxyquinazolin-4-yloxy)benzylphosphonic Acid

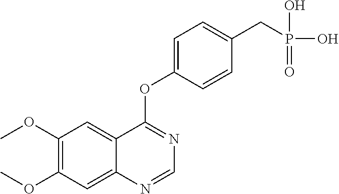 [SMILES: COc1cc2ncnc(Oc3ccc(CP(=O)(O)O)cc3)c2cc1OC]

### Step 1: 4-((6,7-dimethoxyquinazolin-4-yl)oxy)benzaldehyde

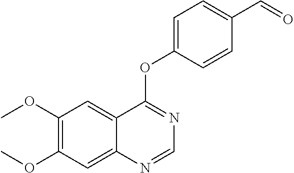 [SMILES: COc1cc2ncnc(Oc3ccc(C=O)cc3)c2cc1OC]

To a stirred mixture of 4-chloro-6,7-dimethoxyquinazoline (1.00 g, 4.452 mmol, 1.00 equiv) and p-hydroxybenzaldehyde (0.82 g, 6.677 mmol, 1.50 equiv) in N,N-dimethylformamide (15 mL) was added cesium carbonate (3.63 g, 11.129 mmol, 2.5 equiv) at room temperature. The resulting mixture was stirred overnight at 85° C. After cooled to room temperature, the solid was collected by filtration, washed with ethyl acetate and water and dried in vacuo to give 800 mg (57%) of 4-((6,7-dimethoxyquinazolin-4-yl)oxy)benzaldehyde as a white solid. ¹H-NMR: (300 MHz, DMSO-d₆, ppm) δ 10.06 (s, 1H), 8.59 (s, 1H), 8.08-8.04 (m, 2H), 7.61-7.58 (m, 3H), 7.42 (s, 1H), 4.01-3.99 (m, 6H).

### Step 2: (4-((6,7-dimethoxyquinazolin-4-yl)oxy)phenyl)methanol

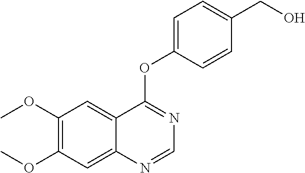 [SMILES: COc1cc2ncnc(Oc3ccc(CO)cc3)c2cc1OC]

To a stirred suspension of 4-((6,7-dimethoxyquinazolin-4-yl)oxy)benzaldehyde (800 mg, 2.527 mmol, 1.00 equiv) in methanol (10 mL) was added sodium borohydride (477 mg, 12.633 mmol, 5.00 equiv) at 0° C. After stirred at room temperature for 3 h, the solid was collected by filtration, washed with methanol and water and dried in vacuo to give 450 mg (51%) of (4-((6,7-dimethoxyquinazolin-4-yl)oxy)phenyl)methanol as a white solid. ¹H-NMR: (300 MHz, DMSO-d₆, ppm) δ 8.54 (s, 1H), 7.58 (s, 1H), 7.44-7.39 (m, 3H), 7.32-7.23 (m, 2H), 5.28-5.20 (m, 1H), 5.55 (d, J=6.0 Hz, 2H), 4.01-3.98 (m, 6H).

### Step 3: 4-(4-(bromomethyl)phenoxy)-6,7-dimethoxyquinazoline

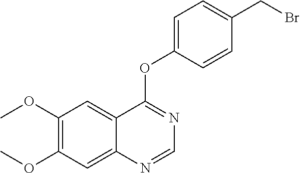 [SMILES: COc1cc2ncnc(Oc3ccc(CBr)cc3)c2cc1OC]

To a stirred mixture of (4-((6,7-dimethoxyquinazolin-4-yl)oxy)phenyl)methanol (450 mg, 90% purity, 1.412 mmol, 1.00 equiv) and triphenylphosphine (1.02 g, 3.890 mmol, 3.00 equiv) in dichloromethane (15 mL) was added N-bromosuccinimide (692 mg, 3.890 mmol, 3.00 equiv) at 0° C. After stirring at room temperature for 5 h, the reaction mixture was concentrated under reduced pressure. The residue was purified by silica gel column chromatography (petroleum ether/ethyl acetate (9:1)) to give 450 mg (71%) of 4-(4-(bromomethyl)phenoxy)-6,7-dimethoxyquinazoline as a red solid. MS (ESI, pos. ion) m/z: 375.1, 377.1 (M+1).

### Step 4: diethyl (4-((6,7-dimethoxyquinazolin-4-yl)oxy)benzyl)phosphonate

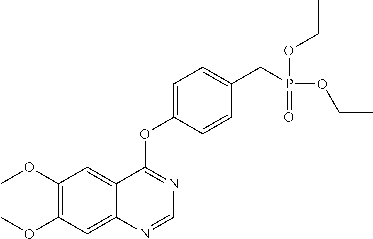 [SMILES: CCOP(=O)(Cc1ccc(Oc2ncnc3cc(OC)c(OC)cc23)cc1)OCC]

To a stirred mixture of 4-(4-(bromomethyl)phenoxy)-6,7-dimethoxyquinazoline (400 mg, 1.066 mmol, 1.00 equiv) in N,N-dimethylformamide (5 mL) was added triethyl phosphite (531 mg, 3.198 mmol, 3.00 equiv) at room temperature. The resulting mixture was stirred overnight at 140° C. After cooled to room temperature, the reaction mixture was quenched with water (10 mL) and extracted with ethyl acetate (50 mL×3). The aqueous phase was concentrated under reduced pressure. The residue was purified by silica gel column chromatography (petroleum ether/ethyl acetate (4:1)) to give 500 mg (75%) of diethyl (4-((6,7-dimethoxyquinazolin-4-yl)oxy)benzyl)phosphonate as a colorless oil. MS (ESI, pos. ion) m/z: 433.2 (M+1).

### Step 5: 4-(6,7-dimethoxyquinazolin-4-yloxy)benzylphosphonic Acid

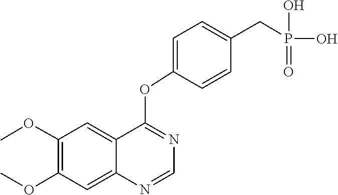 [SMILES: COc1cc2ncnc(Oc3ccc(CP(=O)(O)O)cc3)c2cc1OC]

To a stirred solution of diethyl (4-((6,7-dimethoxyquinazolin-4-yl)oxy)benzyl)phosphonate (450 mg, 78% purity, 0.812 mmol, 1.00 equiv,) in dichloromethane (20 mL) was added bromotrimethylsilane (372 mg, 2.435 mmol, 3.00 equiv) dropwise at room temperature. After stirring overnight, the reaction mixture was concentrated under reduced pressure. The residue was purified by prep-HPLC with the following conditions: Column: XBridge Shield RP18 OBD Column 19×150 mm, 5 um; Mobile Phase A: Water (0.1% FA), Mobile Phase B: acetonitrile; Flow rate: 20 mL/min; Gradient: 20% B to 30% B in 7 min, 220 & 254 nm. The fractions containing the desired product were combined and lyophilized to give 44.8 mg (14%) of 4-(6,7-dimethoxyquinazolin-4-yloxy)benzylphosphonic acid as a white solid. MS (ESI, pos. ion) m/z: 376.8 (M+1). ¹H-NMR: (300 MHz, DMSO-d₆, ppm) δ 8.56 (s, 1H), 7.56 (s, 1H), 7.40-7.30 (m, 3H), 7.22 (d, J=8.1 Hz, 2H), 4.00-3.98 (m, 6H), 3.10 (s, 1H), 3.06 (s, 1H).

### Example 26

### Synthesis of 4-(((6-methoxyquinazolin-4-yl)amino)methyl)phenylboronic Acid

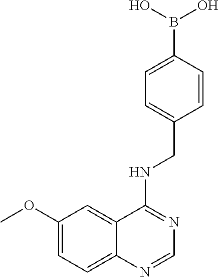 [SMILES: COc1ccc2ncnc(NCc3ccc(B(O)O)cc3)c2c1]

To a solution of 4-chloro-6-methoxyquinazoline (120 mg, 0.617 mmol, 1.00 equiv) in acetonitrile (5 mL) were added 4-(aminomethyl)phenylboronic acid hydrochloride (127 mg, 0.678 mmol, 1.1 equiv) and potassium carbonate (213 mg, 1.541 mmol, 2.50 equiv) at room temperature under nitrogen atmosphere. The resulting mixture was stirred at 80° C. for 16 h. After cooled to room temperature, the solids were filtered and washed with methanol (3 mL×3). The filtrate was concentrated under reduced pressure. The crude product was purified by prep-HPLC with the following conditions: Column: XBridge Prep C18 OBD Column, 19×150 mm, 5 um; Mobile Phase A: Water (0.1% FA), Mobile Phase B: acetonitrile; Flow rate: 25 mL/min; Gradient: 10% B to 40% B in 7 min, 220 & 254 nm. The fractions containing the desired product were combined and lyophilized to give 46.6 mg (23%) of 4-(((6-methoxyquinazolin-4-yl)amino)methyl)phenylboronic acid as a white solid. MS (ESI, pos. ion) m/z: 310.3 (M+1). ¹H-NMR: (300 MHz, DMSO-d₆, ppm) δ 8.69 (t, J=5.7 Hz, 1H), 8.36 (s, 1H), 7.98 (s, 2H), 7.76-7.63 (m, 4H), 7.44-7.31 (m, 3H), 4.81 (d, J=5.4 Hz, 2H), 3.87 (s, 3H).

### Example 27

### Synthesis of 4-(((7-fluoroquinazolin-4-yl)amino)methyl)phenylboronic Acid

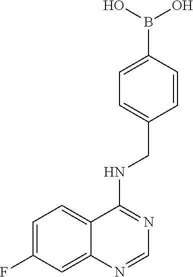 [SMILES: OB(O)c1ccc(CNc2ncnc3cc(F)ccc23)cc1]

To a solution of 4-chloro-7-fluoroquinazoline (80 mg, 0.438 mmol, 1.00 equiv) in acetonitrile (5 mL) were added 4-(aminomethyl)phenylboronic acid hydrochloride (90 mg, 0.482 mmol, 1.10 equiv) and potassium carbonate (151 mg, 1.095 mmol, 2.50 equiv) at room temperature under nitrogen atmosphere. After stirring overnight at 80° C., the reaction mixture was cooled to room temperature and concentrated under reduced pressure. The residue was purified by prep-HPLC with the following conditions: Column: XBridge Prep C18 OBD Column, 19×250 mm, 10 um; Mobile Phase A: Water (10 mmol/L ammonium bicarbonate), Mobile Phase B: acetonitrile; Flow rate: 25 mL/min; Gradient: 38% B to 42% B in 7 min, 220 & 254 nm. The fractions containing the desired product were combined and lyophilized to give 60.7 mg (46%) of 4-(((7-fluoroquinazolin-4-yl)amino)methyl)phenylboronic acid as a white solid. MS (ESI, pos. ion) m/z: 298.2 (M+1). ¹H-NMR (400 MHz, DMSO-d₆, ppm) δ 8.93 (t, J=5.6 Hz, 1H), 8.44-8.39 (m, 2H), 7.96 (s, 2H), 7.73 (d, J=8.0 Hz, 2H), 7.48-7.42 (m, 2H), 7.38-7.30 (m, 2H), 4.79 (d, J=6.0 Hz, 2H). ¹⁹F-NMR (376 MHz, DMSO-d₆, ppm) δ −106.50 (s, 1F).

### Example 28

### Synthesis of 4-(((7-methoxyquinazolin-4-yl)amino)methyl)phenylboronic Acid

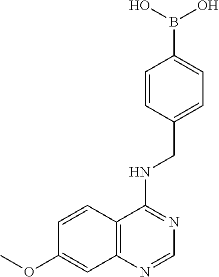 [SMILES: COc1ccc2c(NCc3ccc(B(O)O)cc3)ncnc2c1]

To a solution of 4-chloro-7-methoxyquinazoline (180 mg, 0.928 mmol, 1.00 equiv) in acetonitrile (5 mL) were added 4-(aminomethyl)phenylboronic acid hydrochloride (174 mg, 0.928 mmol, 1.00 equiv) and potassium carbonate (256 mg, 1.856 mmol, 2.00 equiv) at room temperature. After stirring at 80° C. for 3 h, the reaction mixture was filtered and then the filtrate was purified by prep-HPLC with the following conditions: Column: Xbridge Phenyl OBD Column, 19×150 mm, 5 um; Mobile Phase A: Water (0.1% FA), Mobile Phase B: acetonitrile; Flow rate: 25 mL/min; Gradient: 8% B to 15% B in 9 min, 220 & 254 nm. The fractions containing the desired product were combined and lyophilized to give 137.4 mg (48%) of 4-(((7-methoxyquinazolin-4-yl)amino)methyl)phenylboronic acid as a white solid. MS (ESI, pos. ion) m/z: 310.0 (M+1). ¹H-NMR: (300 MHz, DMSO-d₆, ppm) δ 8.64 (s, 1H), 8.38 (s, 2H), 8.22 (d, J=8.4 Hz, 1H), 7.76-7.70 (m, 2H), 7.29 (d, J=6.9 Hz, 2H), 7.13-7.05 (m, 2H), 4.78-4.75 (m, 2H), 3.89 (s, 3H).

### Example 29

### Synthesis of 4-(6,7-dimethoxyquinazolin-4-ylamino)phenethylphosphonic Acid

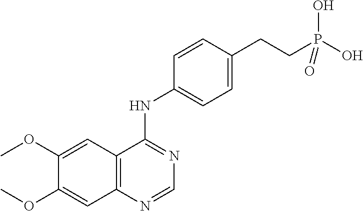 [SMILES: COc1cc2ncnc(Nc3ccc(CCP(=O)(O)O)cc3)c2cc1OC]

### Step 1: diethyl 2-(4-((6,7-dimethoxyquinazolin-4-yl)amino)phenyl)ethylphosphonate

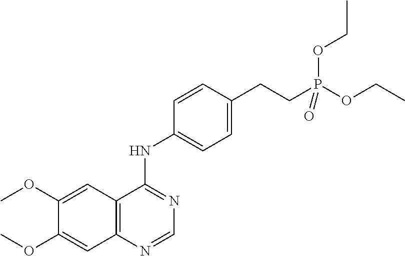 [SMILES: CCOP(=O)(CCc1ccc(Nc2ncnc3cc(OC)c(OC)cc23)cc1)OCC]

A solution of N-(4-(2-bromoethyl)phenyl)-6,7-dimethoxyquinazolin-4-amine (200 mg, 0.515 mmol, 1.00 equiv) in triethyl phosphite (3 mL) was stirred at 140° C. for 3 h. After cooled to room temperature, the reaction mixture was concentrated under reduced pressure. The residue was purified by silica gel column chromatography (petroleum ether/ethyl acetate (1:1)) to afford 100 mg (43%) of diethyl 2-(4-((6,7-dimethoxyquinazolin-4-yl)amino)phenyl)ethylphosphonate as an off-white solid. MS (ESI, pos. ion) m/z: 446.4 (M+1). ¹H-NMR (300 MHz, DMSO-d₆, ppm): δ 9.46 (s, 1H), 8.43 (s, 1H), 7.85 (s, 1H), 7.70 (d, J=8.4 Hz, 2H), 7.29 (d, J=8.4 Hz, 2H), 7.18 (s, 1H), 4.07-3.93 (m, 10H), 2.83-2.74 (m, 2H), 2.14-2.02 (m, 2H), 1.28-1.20 (m, 6H). ³¹P-NMR (121 MHz, DMSO-d₆, ppm): δ 30.596 (s, 1P).

### Step 2: 4-(6,7-dimethoxyquinazolin-4-ylamino)phenethylphosphonic Acid

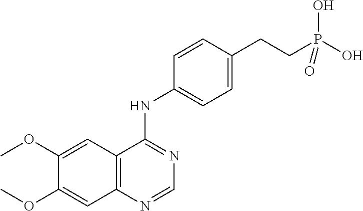 [SMILES: COc1cc2ncnc(Nc3ccc(CCP(=O)(O)O)cc3)c2cc1OC]

To a solution of diethyl 2-(4-((6,7-dimethoxyquinazolin-4-yl)amino)phenyl)ethylphosphonate (100 mg, 0.224 mmol, 1.00 equiv) in dichloromethane (3 mL) was added bromotrimethylsilane (172 mg, 1.122 mmol, 5.00 equiv) dropwise at room temperature. was After stirring at room temperature for 3 h, the reaction mixture was diluted with N,N-dimethylformamide (6 mL) and stirred at room temperature for 1 h. The precipitate was collected by filtration, washed with methanol (15 mL×2) and dried in vacuo to afford 137.4 mg (29%) of 4-(6,7-dimethoxyquinazolin-4-ylamino)phenethylphosphonic acid as an off-white solid. MS (ESI, pos. ion) m/z: 390.3 (M+1). ¹H-NMR (300 MHz, DMSO-d₆, ppm): δ 10.63 (s, 1H), 8.72 (s, 1H), 8.03 (s, 1H), 7.59 (d, J=8.4 Hz, 2H), 7.35 (d, J=8.4 Hz, 2H), 7.23 (s, 1H), 4.04 (s, 6H), 2.90-2.74 (m, 2H), 1.92-1.78 (m, 2H). ³¹P-NMR (121 MHz, DMSO-d₆, ppm): δ 24.72 (s, 1P).

### Example 30

### Synthesis of 4-((7-methoxyquinolin-4-ylamino)methyl)phenylboronic Acid

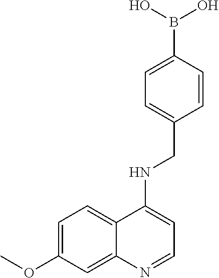 [SMILES: COc1ccc2c(NCc3ccc(B(O)O)cc3)ccnc2c1]

To a solution of 4-chloro-7-methoxyquinoline (100 mg, 0.516 mmol, 1.00 equiv) in isopropanol (3 mL) was added 4-(aminomethyl)phenylboronic acid hydrochloride (145 mg, 0.775 mmol, 1.50 equiv) at room temperature under nitrogen atmosphere. The resulting mixture was irradiated at 120° C. for 1 h in microwave reactor. After cooled to room temperature, the reaction mixture was concentrated under reduced pressure. The crude product was purified by prep-HPLC with the following conditions: Column: XBridge Shield RP18 OBD Column, 19×250 mm, 10 um; Mobile Phase A: Water (10 mmol/L ammonium bicarbonate), Mobile Phase B: acetonitrile; Flow rate: 25 mL/min; Gradient: 30% B to 40% B in 7 min, 220 & 254 nm. The fractions containing the desired product were combined and lyophilized to give 10.4 mg (6%) of 4-((7-methoxyquinolin-4-ylamino)methyl)phenylboronic acid as a white solid. MS (ESI, pos. ion) m/z: 309.3 (M+1). ¹H-NMR (400 MHz, DMSO-d₆, ppm): δ 8.22-8.18 (m, 2H), 8.02-7.84 (m, 3H), 7.73 (d, J=8.0 Hz, 2H), 7.39-7.26 (m, 2H), 7.17-7.05 (m, 2H), 6.26-6.21 (m, 1H), 4.54 (d, J=5.6 Hz, 2H), 3.87 (s, 3H).

### Example 31

### Synthesis of (4-(6,7-dimethoxyquinazolin-4-ylamino)phenoxy)methylboronic Acid

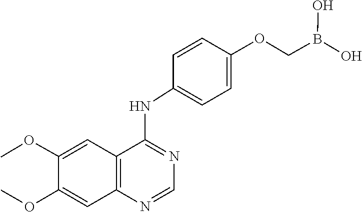 [SMILES: COc1cc2ncnc(Nc3ccc(OCB(O)O)cc3)c2cc1OC]

### Step 1: 4-(6,7-dimethoxyquinazolin-4-ylamino)phenol

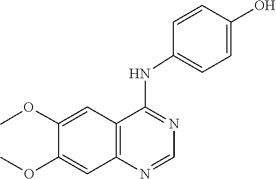 [SMILES: COc1cc2ncnc(Nc3ccc(O)cc3)c2cc1OC]

A mixture of 4-chloro-6,7-dimethoxyquinazoline (1.00 g, 4.452 mmol, 1.00 equiv) and 4-aminophenol (583 mg, 5.342 mmol, 1.20 equiv) in isopropanol (15 mL) was stirred at 85° C. for 2 h. After cooled to room temperature, the solid was collected by filtration, washed with methanol (15 mL×2) and dried in vacuo to give 1.30 g (98%) of 4-(6,7-dimethoxyquinazolin-4-ylamino)phenol as an off-white solid. ¹H-NMR (300 MHz, DMSO-d₆, ppm): δ 11.07 (s, 1H), 9.66 (s, 1H), 8.70 (s, 1H), 8.22 (s, 1H), 7.45-7.40 (m, 2H), 7.32 (s, 1H), 6.88-6.82 (m, 2H), 3.99 (s, 3H), 3.97 (s, 3H).

### Step 2: (4-(6,7-dimethoxyquinazolin-4-ylamino)phenoxy)methylboronic Acid

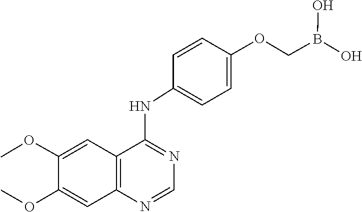 [SMILES: COc1cc2ncnc(Nc3ccc(OCB(O)O)cc3)c2cc1OC]

To a mixture of 4-(6,7-dimethoxyquinazolin-4-ylamino)phenol (100 mg, 0.336 mmol, 1.00 equiv) in dimethyl sulphoxide (3 mL) was added sodium hydride (16 mg, 2.00 equiv) at room temperature. After stirring for 15 min at room temperature, 2-(bromomethyl)-4,4,5,5-tetramethyl-1,3,2-dioxaborolane (111 mg, 0.505 mmol, 1.50 equiv) was added and the resulting mixture was stirred at 50° C. for 2 h. After cooled to room temperature, the reaction mixture was quenched with water (15 mL) and extracted with ethyl acetate (30 mL×2). The combined organic layers were washed with saturated sodium chloride solution (50 mL×2), dried over anhydrous sodium sulfate and filtered. The filtrate was concentrated under reduced pressure. The residue was purified by prep-TLC (methanol:dichloromethane (10:1)) to afford 9.4 mg (7.6%) of (4-(6,7-dimethoxyquinazolin-4-ylamino)phenoxy)methylboronic acid as a yellow solid. MS (ESI, pos. ion) m/z: 356.2 (M+1). ¹H-NMR (300 MHz, DMSO-d₆, ppm): δ 8.44-8.21 (m, 1H), 7.33-7.12 (m, 4H), 6.96-6.80 (m, 3H), 6.40-6.31 (m, 1H), 3.92-3.78 (m, 5H), 3.28-3.11 (m, 3H).

### Example 32

### Synthesis of 4-(6,7-dimethoxyquinazolin-4-ylamino)phenoxymethylphosphonic Acid

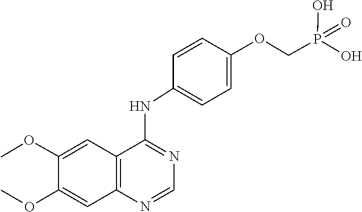 [SMILES: COc1cc2ncnc(Nc3ccc(OCP(=O)(O)O)cc3)c2cc1OC]

### Step 1: diethyl (4-(6,7-dimethoxyquinazolin-4-ylamino)phenoxy)methylphosphonate

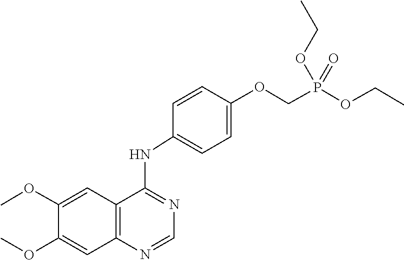 [SMILES: CCOP(=O)(COc1ccc(Nc2ncnc3cc(OC)c(OC)cc23)cc1)OCC]

To a mixture of 4-(6,7-dimethoxyquinazolin-4-ylamino)phenol (300 mg, 1.009 mmol, 1.00 equiv) in dimethyl sulphoxide (15 mL) were added cesium carbonate (658 mg, 2.018 mmol, 2.00 equiv) at room temperature. After stirring at room temperature for 1 h, diethyl bromomethylphosphonate (466 mg, 2.018 mmol, 2.00 equiv) was added and the resulting mixture was stirred overnight at 50° C. After cooled to room temperature, the reaction mixture was quenched with water (50 mL) and extracted with ethyl acetate (60 mL×2). The combined organic layers were washed with saturated sodium chloride solution (100 mL×2), dried over anhydrous sodium sulfate and filtered. The filtrate was concentrated under reduced pressure. The residue was purified by silica gel column chromatography (petroleum ether/ethyl acetate (1:1)) to afford 250 mg (55%) of diethyl 4-((6,7-dimethoxyquinazolin-4-yl)amino)phenoxymethylphosphonate as a grey solid. MS (ESI, pos. ion) m/z: 470.3 (M+Na).

### Step 2: 4-(6,7-dimethoxyquinazolin-4-ylamino)phenoxymethylphosphonic Acid

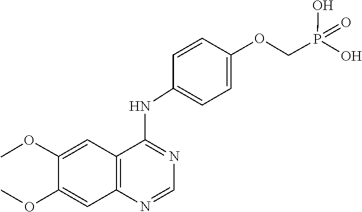 [SMILES: COc1cc2ncnc(Nc3ccc(OCP(=O)(O)O)cc3)c2cc1OC]

To a solution of diethyl 4-((6,7-dimethoxyquinazolin-4-yl)amino)phenoxymethylphosphonate (250 mg, 0.559 mmol, 1.00 equiv) in tetrahydrofuran (10 mL) was added bromotrimethylsilane (257 mg, 1.676 mmol, 3.00 equiv) dropwise at 0 C. After stirring overnight at 50° C., the reaction mixture was quenched with methanol (15 mL). The precipitate was collected by filtration, washed with methanol (15 mL×2) and dried in vacuo to give 207.4 mg (93%) of 4-(6,7-dimethoxyquinazolin-4-ylamino)phenoxymethylphosphonic acid as an off-white solid. MS (ESI, pos. ion) m/z: 392.3 (M+1). ¹H-NMR (300 MHz, DMSO-d₆, ppm): δ 11.06 (s, 1H), 8.82 (s, 1H), 8.09 (s, 1H), 7.54 (d, J=9.0 Hz, 2H), 7.23 (s, 1H), 7.15 (d, J=9.0 Hz, 2H), 4.16 (d, J=10.2 Hz, 2H), 4.01-3.85 (m, 8H). ³¹P-NMR (121 MHz, DMSO-d₆, ppm): δ 14.54 (s, 1P).

### Example 33

### Synthesis of 2-(4-((6,7-Dimethoxyquinazolin-4-yl)amino)phenyl)ethylboronic Acid Formic Acid Salt

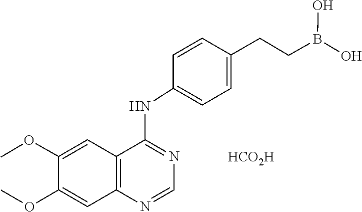 [SMILES: COc1cc2ncnc(Nc3ccc(CCB(O)O)cc3)c2cc1OC ; "HCO2H"]

### Step 1: 2-(4-((6,7-dimethoxyquinazolin-4-yl)amino)phenyl)ethanol

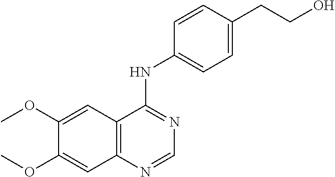 [SMILES: COc1cc2ncnc(Nc3ccc(CCO)cc3)c2cc1OC]

To a solution of 4-chloro-6,7-dimethoxyquinazoline (5.00 g, 22.258 mmol, 1.00 equiv) in isopropanol (80 mL) was added p-aminophenylethanol (3.66 g, 26.709 mmol, 1.20 equiv) at room temperature. The resulting mixture was stirred at 85° C. for 2 h. After cooled to room temperature, the solid was collected by filtration and dried in vacuo to give 7.00 g (90%) of 2-(4-((6,7-dimethoxyquinazolin-4-yl)amino)phenyl)ethanol as a yellow solid. ¹H-NMR (300 MHz, DMSO-d₆, ppm): δ 14.70 (brs, 1H), 11.34 (s, 1H), 8.79 (s, 1H), 8.29 (s, 1H), 7.57-7.54 (m, 2H), 7.34-7.31 (m, 3H), 4.01-3.99 (m, 6H), 3.64 (t, J=6.0 Hz, 2H), 2.77 (t, J=6.0 Hz, 2H).

### Step 2: N-(4-(2-bromoethyl)phenyl)-6,7-dimethoxyquinazolin-4-amine

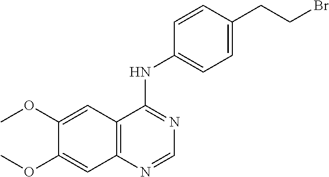 [SMILES: COc1cc2ncnc(Nc3ccc(CCBr)cc3)c2cc1OC]

To a solution of 2-(4-((6,7-dimethoxyquinazolin-4-yl)amino)phenyl)ethanol (400 mg, 1.229 mmol, 1.00 equiv, 98% purity) in dichloromethane (10 mL) were added carbon tetrabromide (815 mg, 2.459 mmol, 2.00 equiv) and triphenylphosphine (645 mg, 2.459 mmol, 2.00 equiv). After stirring for 12 h at room temperature, the reaction mixture was concentrated under reduced pressure. The residue was purified by silica gel column chromatography (dichloromethane/methanol (95:5)) to give 600 mg (75%) of N-(4-(2-bromoethyl)phenyl)-6,7-dimethoxyquinazolin-4-amine as a yellow solid. MS (ESI, pos. ion) m/z: 387.7 (M+1).

### Step 3: 6,7-dimethoxy-N-(4-(2-(4,4,5,5-tetramethyl-1,3,2-dioxaborolan-2-yl)ethyl)phenyl)quinazolin-4-amine

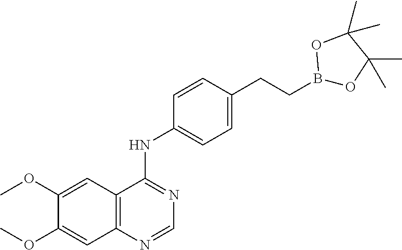

To a solution of N-(4-(2-bromoethyl)phenyl)-6,7-dimethoxyquinazolin-4-amine (500 mg, 1.160 mmol, 1.00 equiv, 90% purity) in N,N-dimethylformamide (10 mL) were added bis(pinacolato)diboron (589 mg, 0.320 mmol, 2.00 equiv), lithium methanolate (132 mg, 3.480 mmol, 3.00 equiv), triphenylphosphine (37 mg, 0.139 mmol, 0.12 equiv) and copper(I) iodide (22 mg, 0.116 mmol, 0.10 equiv) at room temperature under argon atmosphere. The resulting mixture was stirred at 35° C. for 12 h. After cooled to room temperature, the reaction mixture was filtered. The filtrate was concentrated under reduced pressure to give 300 mg (34%) of 6,7-dimethoxy-N-(4-(2-(4,4,5,5-tetramethyl-1,3,2-dioxaborolan-2-yl)ethyl)phenyl)quinazolin-4-amine as a yellow solid. MS (ESI, pos. ion) m/z: 436.4 (M+1).

### Step 4: 2-(4-((6,7-dimethoxyquinazolin-4-yl)amino)phenyl)ethylboronic Acid Formic Acid Salt

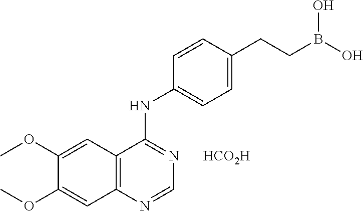 [SMILES: COc1cc2ncnc(Nc3ccc(CCB(O)O)cc3)c2cc1OC ; "HCO2H"]

To a solution of 6,7-dimethoxy-N-(4-(2-(4,4,5,5-tetramethyl-1,3,2-dioxaborolan-2-yl)ethyl)phenyl)quinazolin-4-amine (300 mg, 0.400 mmol, 1.00 equiv, 58% purity) in tetrahydrofuran (4 mL) and water (1 mL) were added sodium periodate (428 mg, 2.000 mmol, 5.00 equiv) at room temperature. After stirring for 15 min, 2M hydrochloric acid (3.2 mL) was added. The resulting mixture was stirred for 2 h at room temperature and filtered. The filtrate was purified by prep-HPLC with the following conditions: Column: XBridge Prep C18 OBD Column, 19×150 mm, 5 um; Mobile Phase A: Water (0.05% TFA), Mobile Phase B: Methanol; Flow rate: 25 mL/min; Gradient: 19% B to 27% B in 7 min, 220 & 254 nm. The fractions containing the desired product were combined and lyophilized to give 30.9 mg (19%) of 2-(4-((6,7-dimethoxyquinazolin-4-yl)amino)phenyl)ethylboronic acid formic acid salt as a yellow solid. MS (ESI, pos. ion) m/z: 354.3 (M+1). ¹H-NMR (300 MHz, DMSO-d₆, ppm): δ 14.70 (brs, 1H), 11.19 (s, 1H), 8.80 (s, 1H), 8.21 (s, 1H), 7.64-7.51 (m, 3H), 7.32-7.29 (m, 3H), 4.01 (s, 6H), 2.74-2.67 (m, 2H), 0.99-0.93 (m, 2H).

### Example 34

### Synthesis of 4-((quinazolin-4-ylamino)methyl)phenylboronic Acid

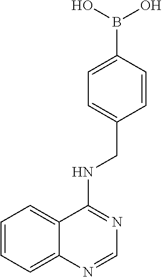 [SMILES: OB(O)c1ccc(CNc2ncnc3ccccc23)cc1]

To a solution of 4-(aminomethyl)phenylboronic acid hydrochloride (137 mg, 0.729 mmol, 1.20 equiv) in acetonitrile (5 mL) was added potassium carbonate (252 mg, 1.823 mmol, 3.00 equiv) at room temperature. After stirring for 20 min, 4-chloroquinazoline (100 mg, 0.608 mmol, 1.00 equiv) was added. The resulting mixture was stirred at 80° C. for 2 h. After cooled to room temperature, the reaction mixture was filtered through celite. The filtrate was purified by prep-HPLC with the following conditions: Column: XBridge Shield RP18 OBD Column, 19×250 mm, 10 um; Mobile Phase A: Water (0.1% FA), Mobile Phase B: Acetonitrile; Flow rate: 25 mL/min; Gradient: 10% B to 10% B in 7 min, 220 & 254 nm. The fractions containing the desired product were combined and lyophilized to give 24.5 mg (14%) of 4-((quinazolin-4-ylamino)methyl)phenylboronic acid as a white solid. MS (ESI, pos. ion) m/z: 280.2 (M+1). ¹H-NMR (300 MHz, DMSO-d₆, ppm): δ 8.87-8.79 (m, 1H), 8.45 (s, 1H), 8.32 (d, J=8.4 Hz, 1H), 7.99 (s, 2H), 7.86-7.68 (m, 4H), 7.57-7.50 (m, 1H), 7.38-7.28 (m, 2H), 4.81 (d, J=5.4 Hz, 2H).

### Example 35

### Synthesis of 4-(((7-chloroquinazolin-4-yl)amino)methyl)phenylboronic Acid Formic Acid Salt

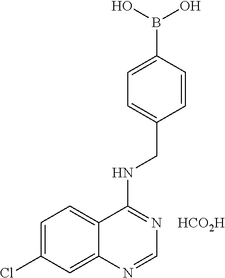 [SMILES: OB(O)c1ccc(CNc2ncnc3cc(Cl)ccc23)cc1 ; "HCO2H"]

To a stirred solution of 4,7-dichloroquinazoline (100 mg, 0.479 mmol, 1.00 equiv) in acetonitrile (10 mL) were added potassium carbonate (139 mg, 1.005 mmol, 2.00 equiv) and 4-(aminomethyl)phenylboronic acid hydrochloride (104 mg, 0.553 mmol, 1.10 equiv). After stirring overnight at 80° C., the reaction mixture was cooled to room temperature and concentrated under reduced pressure. The crude product was purified by prep-HPLC with the following conditions: Column: XBridge Prep OBD C18 Column, 19×250 mm, 5 um; Mobile Phase A: Water (0.1% FA), Mobile Phase B: acetonitrile; Flow rate: 25 mL/min; Gradient: 17% B to 17% B in 7 min, 220 & 254 nm. The fractions containing the desired product were combined and lyophilized to give 33.1 mg (18%) of 4-(((7-chloroquinazolin-4-yl)amino)methyl)phenylboronic acid formic acid salt as an off-white solid. MS (ESI, pos. ion) m/z: 314.2 (M+1). ¹H-NMR (300 MHz, DMSO-d₆, ppm): δ 9.01-8.98 (s, 1H), 8.46 (s, 1H), 8.36 (d, J=8.7 Hz, 1H), 8.15 (s, 0.31H), 8.02 (s, 2H), 7.75-7.72 (m, 3H), 7.62-7.59 (m, 1H), 7.30-7.28 (m, 2H), 4.79 (d, J=5.4 Hz, 2H).

### Example 36

### Synthesis of 4-(((8-hydroxyquinazolin-4-yl)amino)methyl)phenylboronic Acid

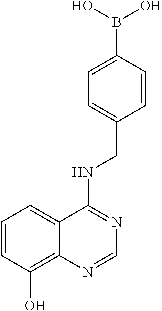 [SMILES: OB(O)c1ccc(CNc2ncnc3c(O)cccc23)cc1]

### Step 1: 4-(((8-methoxyquinazolin-4-yl)amino)methyl)phenylboronic Acid

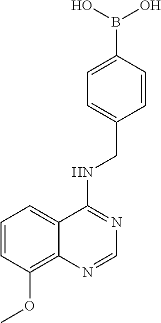 [SMILES: COc1cccc2c(NCc3ccc(B(O)O)cc3)ncnc12]

To a stirred solution of 4-chloro-8-methoxyquinazoline (100 mg, 0.514 mmol, 1.00 equiv) in acetonitrile (10 mL) were added potassium carbonate (142 mg, 1.028 mmol, 2.00 equiv) and 4-(aminomethyl)phenylboronic acid hydrochloride (116 mg, 0.617 mmol, 1.20 equiv). After stirring overnight at 80° C., the reaction mixture was diluted with methanol (10 mL). The solids were filtered and the filter cake was washed with acetonitrile (10 mL×2). The filtrate was concentrated under reduced pressure to afford 120 mg (62.71%) of 4-(((8-methoxyquinazolin-4-yl)amino)methyl)phenylboronic acid as an off-white solid. MS (ESI, pos. ion) m/z: 309.8 (M+1).

### Step 2: 4-(((8-hydroxyquinazolin-4-yl)amino)methyl)phenylboronic Acid

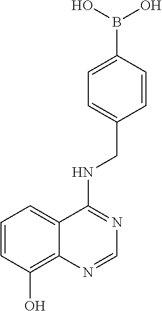 [SMILES: OB(O)c1ccc(CNc2ncnc3c(O)cccc23)cc1]

To a stirred mixture of 4-(((8-methoxyquinazolin-4-yl)amino)methyl)phenylboronic acid (80 mg, 0.259 mmol, 1.00 equiv) in dichloromethane (5 mL) was added phosphorus tribromide (701 mg, 2.588 mmol, 10.00 equiv) dropwise at room temperature. After stirring overnight, the reaction mixture was quenched by addition of methanol (5 mL) at 0° C. The resulting mixture was concentrated under vacuum. The crude product was purified by prep-HPLC with the following conditions: Column: XBridge Prep OBD C18 Column, 19×250 mm, 5 um; Mobile Phase A: Water (0.1% FA), Mobile Phase B: acetonitrile; Flow rate: 25 mL/min; Gradient: 14% B to 14% B in 7 min, 220 & 254 nm. The fractions containing the desired product were combined and lyophilized to give 33 mg (36%) of 4-(((8-hydroxyquinazolin-4-yl)amino)methyl)phenylboronic acid as an off-white solid. MS (ESI, pos. ion) m/z: 296.3 (M+1). ¹H-NMR (300 MHz, DMSO-d₆, ppm): δ 8.77 (brs, 1H), 8.45 (s, 2H), 8.30-8.25 (m, 3H), 7.73-7.71 (m, 2H), 7.38-7.24 (m, 3H), 7.17-7.10 (m, 1H), 4.80 (d, J=5.7 Hz, 2H).

### Example 37

### Synthesis of 4-(((7-methoxyquinazolin-4-yl)amino)methyl)phenylphosphonic Acid Formic Acid Salt

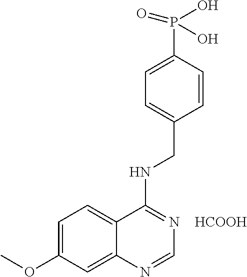 [SMILES: COc1ccc2c(NCc3ccc(P(=O)(O)O)cc3)ncnc2c1 ; "HCOOH"]

### Step 1: tert-butyl N-((4-bromophenyl)methyl)carbamate

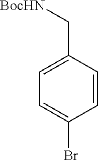 [SMILES: CC(C)(C)OC(=O)NCc1ccc(Br)cc1]

To a stirred mixture of 4-bromo-benzylamine (2.00 g, 10.750 mmol, 1.00 equiv) in water (20 mL) were added di-tert-butyl dicarbonate (2.58 g, 11.821 mmol, 1.10 equiv) and sodium hydroxide (1.29 g, 32.249 mmol, 3.00 equiv) at room temperature. After stirring at room temperature for 16 h, the reaction mixture was diluted with ethyl acetate (100 mL). The organic layer was separated and the aqueous layer was extracted with ethyl acetate (200 mL×2). The combined organic layers were washed with brine (200 mL), dried over anhydrous sodium sulfate and filtered. The filtrate was concentrated under reduced pressure to afford 2.80 (91%) of tert-butyl N-((4-bromophenyl)methyl)carbamate as a white solid. ¹H-NMR (400 MHz, DMSO-d₆, ppm): δ 7.53-7.50 (m, 2H), 7.43 (t, J=6.0 Hz, 1H), 7.21-7.07 (m, 2H), 4.09 (d, J=6.0 Hz, 2H), 1.39 (s, 9H).

### Step 2: 4-(aminomethyl)phenylphosphonic Acid Hydrochloride

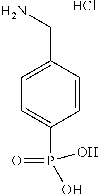 [SMILES: Cl ; NCc1ccc(P(=O)(O)O)cc1]

To a solution of tert-butyl N-((4-bromophenyl)methyl)carbamate (1.00 g, 3.494 mmol, 1.00 equiv) and diethyl phosphonate (2.41 g, 17.472 mmol, 5.00 equiv) in triethylamine (5 mL) was added tetrakis(triphenylphosphine)palladium (0.81 g, 0.699 mmol, 0.20 equiv) at room temperature. After stirring at 100° C. for 16 h under argon atmosphere, the reaction mixture was cooled to room temperature. The solid was filtered out and the filtrate was concentrated under reduced pressure. The residue was dissolved in ethyl acetate (100 mL), washed with 0.5 N hydrochloric acid aqueous solution (50 mL×2), 0.5 N sodium hydroxide aqueous solution (50 mL×2), water (100 mL) and brine (100 mL), dried over anhydrous sodium sulfate, filtered and concentrated under reduced pressure. The residue was dissolved in 2 M hydrochloric (gas) in ethyl acetate (10 mL) and stirred for 2 h at room temperature. The mixture was evaporated to dryness under reduced pressure to give 1.3 g of crude product, which was suspended in acetonitrile (10 mL) and added trimethylsilyl bromide (2.67 g, 17.472 mmol, 5.00 equiv) at 0° C. After stirring for 16 h at room temperature, the reaction mixture was concentrated under reduced pressure to removed acetonitrile. The residue was dissolved in water (10 mL) and extracted with ethyl acetate (10 mL×3). The aqueous was evaporated to dryness to give the crude product, which was triturated with acetonitrile (20 mL) for 30 minutes at room temperature, the solid was collected by filtration, washed with acetonitrile (10 mL×2), and dried under vacuum to give 0.42 g, (53.8%) 4-(aminomethyl)phenylphosphonic acid hydrochloride as a light yellow solid. ¹H-NMR (400 MHz, DMSO-d₆, ppm): δ 8.25 (brs, 3H), 7.73-7.64 (m, 2H), 7.55-7.53 (m, 2H), 4.11-4.07 (m, 2H).

### Step 3: 4-(((7-methoxyquinazolin-4-yl)amino)methyl)phenylphosphonic Acid Formic Acid Salt

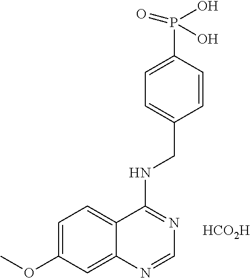 [SMILES: COc1ccc2c(NCc3ccc(P(=O)(O)O)cc3)ncnc2c1 ; "HCO2H"]

This compound was synthesized by the same method as described in Example 35 except 4-(aminomethyl)phenylphosphonic acid hydrochloride (116 mg, 0.519 mmol, 1.00 equiv) and 4-chloro-7-methoxyquinazoline (101 mg, 0.519 mmol, 1.00 equiv) were used. Yield: 20.9 mg (9.44%). MS (ESI, pos. ion) m/z: 346.2 (M+1). ¹H-NMR (300 MHz, DMSO-d₆, ppm): δ 8.55-8.15 (m, 4H), 7.65-7.62 (m, 2H), 7.44-7.40 (m, 2H), 7.14-7.10 (m, 2H), 4.82 (s, 2H), 3.93 (s, 3H). ³¹P-NMR (121 MHz, DMSO-d₆, ppm): δ −12.27 (s, 1P).

### Example 38

### Synthesis of 4-(((7-hydroxyquinazolin-4-yl)amino)methyl)phenylboronic Acid

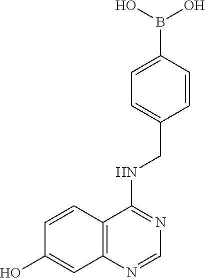 [SMILES: OB(O)c1ccc(CNc2ncnc3cc(O)ccc23)cc1]

This compound was synthesized by the same method as described in Example 35 except 4-chloroquinazolin-7-ol (100 mg, 0.554 mmol, 1.00 equiv) was used. Yield: 17.6 mg (10%). MS (ESI, pos. ion) m/z: 296.2 (M+1). ¹H-NMR (300 MHz, DMSO-d₆, ppm): δ 8.59 (t, J=5.4 Hz, 1H), 8.32 (s, 1H), 8.14 (d, J=8.7 Hz, 2H), 7.99 (brs, 2H), 7.73 (d, J=8.1 Hz, 2H), 7.29 (d, J=8.1 Hz, 2H), 7.05-6.93 (m, 2H), 4.76 (d, J=5.7 Hz, 2H).

### Example 39

### Synthesis of 4-(((2-amino-7-methoxyquinazolin-4-yl)amino)methyl)phenylboronic Acid

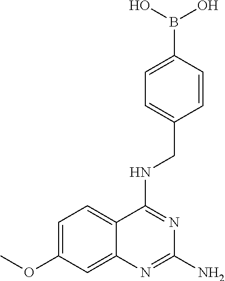 [SMILES: COc1ccc2c(NCc3ccc(B(O)O)cc3)nc(N)nc2c1]

### Step 1: 4-(((2-chloro-7-methoxyquinazolin-4-yl)amino)methyl)phenylboronic Acid

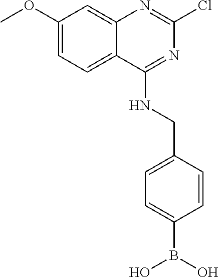 [SMILES: COc1ccc2c(NCc3ccc(B(O)O)cc3)nc(Cl)nc2c1]

To a solution of 2,4-dichloro-7-methoxyquinazoline (600 mg, 2.62 mmol, 1.00 equiv.) in acetonitrile (20 mL) were added 4-(aminomethyl)phenylboronic acid hydrochloride (589 mg, 3.14 mmol, 1.20 equiv.) and N,N-diisopropylethylamine (846 mg, 6.55 mmol, 2.50 equiv.) at 0 C. After stirring overnight at room temperature, the reaction mixture was concentrated under reduced pressure. The residue was dissolved in acetonitrile (10 mL) and diethyl ether (50 mL) and stirred overnight at room temperature. The precipitated solid was collected by filtration, washed with diethyl ether (10 mL×3) and dried to afford 680 mg (73%) of 4-(((2-chloro-7-methoxyquinazolin-4-yl)amino)methyl)phenylboronic acid as a white solid. MS (ESI, pos. ion) m/z: 344.2 (M+1).

### Step 2: 4-(((2-amino-7-methoxyquinazolin-4-yl)amino)methyl)phenylboronic Acid

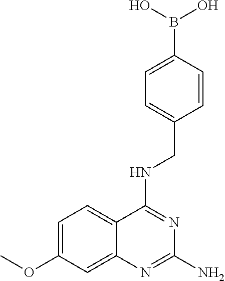 [SMILES: COc1ccc2c(NCc3ccc(B(O)O)cc3)nc(N)nc2c1]

To a suspension of 4-(((2-chloro-7-methoxyquinazolin-4-yl)amino)methyl)phenylboronic acid (200 mg, 0.58 mmol, 1.00 equiv.) in isopropanol (4 mL) were added trifluoroacetic acid (266 mg, 2.33 mmol, 4.00 equiv.) and ammonia (0.25 mL, 1.75 mmol, 7.0 M in methanol, 2.00 equiv.) slowly at room temperature, respectively. After irradiated at 120° C. for 2 h with microwave reactor, the reaction mixture was cooled to room temperature. The solids were filtered off and washed with acetonitrile (10 mL). The filtrate was concentrated under reduced pressure. The residue was purified by prep-HPLC with the following conditions: Column: XBridge Shield RP18 OBD Column, 19×250 mm, 10 um; Mobile Phase A: Water (0.1% FA), Mobile Phase B: acetonitrile; Flow rate: 25 mL/min; Gradient: 17% B to 17% B in 8 min, 220 & 254 nm. The fractions containing the desired product were combined and lyophilized to give 11.3 mg (6%) of 4-(((2-amino-7-methoxyquinazolin-4-yl)amino)methyl)phenylboronic acid as an off-white solid. MS (ESI, pos. ion) m/z: 325.0 (M+1). ¹H-NMR (400 MHz, DMSO-d₆, ppm): δ 8.45 (s, 1H), 8.19 (s, 1H), 8.05-7.95 (m, 3H), 7.73 (d, J=8.0 Hz, 2H), 7.30 (d, J=8.0 Hz, 2H), 6.67-6.60 (m, 2H), 7.40 (s, 1H), 4.73 (d, J=5.2 Hz, 2H), 3.81 (s, 3H).

### Example 40

### Synthesis of 4-(((7-methoxy-2-(methylamino)quinazolin-4-yl)amino)methyl)phenylboronic Acid

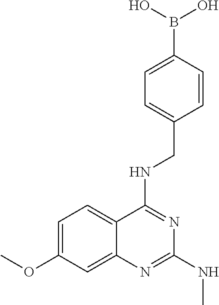

To a suspension of 4-(((2-chloro-7-methoxyquinazolin-4-yl)amino)methyl)phenylboronic acid (100 mg, 0.29 mmol, 1.00 equiv.) in 1,4-dioxane (5 mL) were added triethylamine (88 mg, 0.87 mmol, 3.00 equiv.) and methylamine hydrochloride (39 mg, 0.58 mmol, 2.00 equiv.) at room temperature. After stirring for 16 hours at 110° C., the mixture was cooled to room temperature, filtered and washed with acetonitrile (15 mL). The filtrate was concentrated under reduced pressure and the residue was purified by prep-HPLC with the following conditions: Column: XBridge Shield RP18 OBD Column, 19×250 mm, 10 um; Mobile Phase A: Water (0.1% FA), Mobile Phase B: acetonitrile; Flow rate: 25 mL/min; Gradient: 20% B to 30% B in 7 min, 220 & 254 nm. The fractions containing the desired product were combined and lyophilized to give 28.9 mg (28%) of 4-(((7-methoxy-2-(methylamino)quinazolin-4-yl)amino)methyl)phenylboronic acid as an off-white solid. MS (ESI, pos. ion) m/z: 338.0 (M+1). ¹H-NMR (400 MHz, DMSO-d₆, ppm): δ 8.16 (s, 2H), 8.00 (brs, 2H), 7.93 (d, J=8.8 Hz, 1H), 7.72 (d, J=8.0 Hz, 2H), 7.30 (d, J=8.0 Hz, 2H), 6.67-6.63 (m, 2H), 4.69 (d, J=5.6 Hz, 2H), 3.81 (s, 3H), 2.78 (d, J=4.8 Hz, 3H).

### Example 41

### Synthesis of 4-(((2-(dimethylamino)-7-methoxyquinazolin-4-yl)amino)methyl)phenylboronic Acid Formic Acid Salt

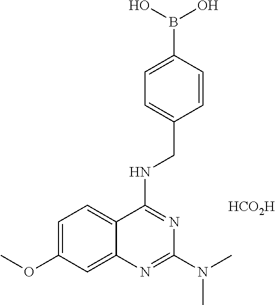

This compound was synthesized by the same method as described in Example 40 except dimethylamine hydrochloride (47 mg, 0.58 mmol, 2.00 equiv.) was used. Yield: 50.3 mg (42%). MS (ESI, pos. ion) m/z: 353.3 (M+1). ¹H-NMR (400 MHz, DMSO-d₆, ppm): δ 8.42 (brs, 1H), 8.15 (s, 1H), 7.98-7.92 (m, 3H), 7.71 (d, J=8.0 Hz, 2H), 7.32 (d, J=8.0 Hz, 2H), 6.70-6.64 (m, 2H), 4.68 (d, J=5.6 Hz, 2H), 3.81 (s, 3H), 3.07 (s, 6H).

### Example 42

### Synthesis of 4-(((7-hydroxy-6-methoxyquinazolin-4-yl)amino)methyl)phenylboronic Acid Formic Acid Salt

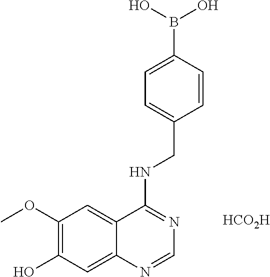 [SMILES: COc1cc2c(NCc3ccc(B(O)O)cc3)ncnc2cc1O ; "HCO2H"]

This compound was synthesized by the same method as described in Example 35 except 4-chloro-6-methoxyquinazolin-7-ol (150 mg, 0.71 mmol, 1.00 equiv.) was used. Yield: 42 mg (15%). MS (ESI, pos. ion) m/z: 326.3 (M+1). ¹H-NMR (400 MHz, DMSO-d₆, ppm): δ 10.15 (s, 1H), 8.38 (t, J=5.6 Hz, 1H), 8.25 (s, 1H), 8.15 (s, 1H), 7.98 (brs, 2H), 7.73 (d, J=7.6 Hz, 2H), 7.65 (s, 1H), 7.30 (d, J=8.0 Hz, 2H), 6.97 (s, 1H), 4.77 (d, J=5.6 Hz, 2H), 3.89 (s, 3H).

### Example 43

### Synthesis of 4-(((6-hydroxy-7-methoxyquinazolin-4-yl)amino)methyl)phenylboronic Acid

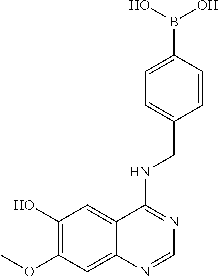 [SMILES: COc1cc2ncnc(NCc3ccc(B(O)O)cc3)c2cc1O]

This compound was synthesized by the same method as described in Example 35 except 4-chloro-7-methoxyquinazolin-6-ol (100 mg, 0.475 mmol, 1.00 equiv) was used. Yield: 36.2 mg (23%). MS (ESI, pos. ion) m/z: 326.0 (M+1). ¹H-NMR (400 MHz, DMSO-d₆, ppm): δ 9.47 (s, 1H), 8.31 (t, J=6.0 Hz, 1H), 8.25 (s, 1H), 7.96 (s, 2H), 7.71 (d, J=8.0 Hz, 2H), 7.53 (s, 1H), 7.28 (d, J=8.0 Hz, 2H), 7.10 (s, 1H), 4.73 (d, J=6.0 Hz, 2H), 3.93 (s, 3H).

### Example 44 Synthesis of 4-(((7,8-dimethoxyquinazolin-4-yl)amino)methyl)phenylboronic Acid

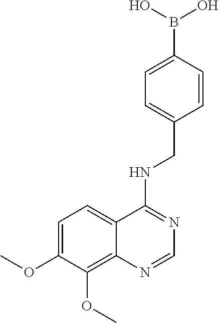 [SMILES: COc1ccc2c(NCc3ccc(B(O)O)cc3)ncnc2c1OC]

The title compound was synthesized by the same method as described in Example 35 except cesium carbonate (435 mg, 1.335 mmol, 3.00 equiv) and 4-chloro-7,8-dimethoxyquinazoline (100 mg, 0.445 mmol, 1.00 equiv) were used. Yield: 48.9 mg (32%). MS (ESI, pos. ion) m/z: 340.3 (M+1). ¹H-NMR (400 MHz, DMSO-d₆, ppm): δ 8.77 (t, J=6.0 Hz, 1H), 8.39 (s, 1H), 8.10-8.04 (m, 1H), 7.98 (s, 2H), 7.87 (d, J=8.1 Hz, 2H), 7.41-7.29 (m, 3H), 4.78 (d, J=5.7 Hz, 2H), 3.94 (s, 3H), 3.88 (s, 3H).

### Example 45

### Synthesis of 4-(((8-aminoquinazolin-4-yl)amino)methyl)phenylboronic Acid Formic Acid Salt

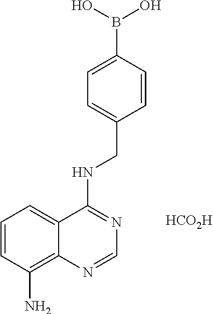 [SMILES: Nc1cccc2c(NCc3ccc(B(O)O)cc3)ncnc12 ; "HCO2H"]

### Step 1: 4-(((8-nitroquinazolin-4-yl)amino)methyl)phenylboronic Acid

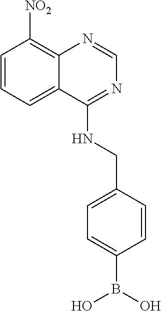 [SMILES: O=[N+]([O-])c1cccc2c(NCc3ccc(B(O)O)cc3)ncnc12]

This compound was synthesized by the same method as described in Example 35 except 4-chloro-8-nitroquinazoline (180 mg, 0.859 mmol, 1.00 equiv) was used. Yield: 195 mg (59%). MS (ESI, pos. ion) m/z: 325.2 (M+1).

### Step 2: 4-(((8-aminoquinazolin-4-yl)amino)methyl)phenylboronic Acid Formic Acid Salt

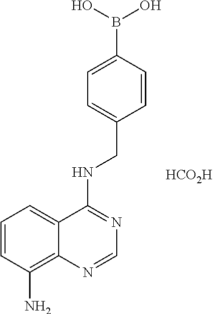 [SMILES: Nc1cccc2c(NCc3ccc(B(O)O)cc3)ncnc12 ; "HCO2H"]

To a solution of 4-(((8-nitroquinazolin-4-yl)amino)methyl)phenylboronic acid (170 mg, 1 equiv) in methanol (6 mL) was added 10% Pd/C (34 mg). The resulting mixture was stirred at room temperature for 3 h under hydrogen atmosphere (1-2 atm.). The reaction mixture was filtered through a Celite. The filtrate was purified by prep-HPLC with the following conditions: Column: XBridge Shield RP18 OBD Column, 19×250 mm, 10 um; Mobile Phase A: Water (0.1% FA), Mobile Phase B: acetonitrile; Flow rate: 25 mL/min; Gradient: 13% B to 13% B in 6 min, 220 & 254 nm. The fractions containing the desired product were combined and lyophilized to give 96.6 mg (53%) of 4-(((8-aminoquinazolin-4-yl)amino)methyl)phenylboronic acid formic acid salt. MS (ESI, pos. ion) m/z: 295.1 (M+1). ¹H-NMR (300 MHz, DMSO-d₆, ppm): δ 12.71 (brs, 1H), 8.58 (s, 1H), 8.36 (s, 1H), 8.15 (s, 1H), 7.97 (s, 2H), 7.73 (d, J=7.8 Hz, 2H), 7.43-7.18 (m, 4H), 6.90 (d, J=7.8 Hz, 1H), 5.78 (s, 2H), 4.78 (d, J=5.4 Hz, 2H).

### BIOLOGICAL EXAMPLES

### Example 1

### Measurement of pNP-TMP Hydrolysis by ENPP1

p-Nitrophenyl thymidine 5′-monophosphate (pNP-TMP) is a synthesized substrate for ENPP1. The ENPP1 enzyme activity assay with pNP-TMP substrate was conducted as follows:

First, in a 60 μl reaction, 7.5 ng purified ENPP1 was mixed with compounds of Formula (I) (test compound) ranging from 13.7 pM to 10 μM. Incubation of ENPP1 with compounds was set at 25° C. for 10 min. Reactions with DMSO only (with ENPP1 but no compound) gave the fastest reaction (MAX Activity). For each compound dilution, wells with assay buffer (50 mM Tris-HCl, pH8.8, 250 mM NaCl, 0.1 mg/ml BSA, 1% DMSO) only but no ENPP1 were included as controls for subtraction of test compound derived absorbance at 405 nm.

Second, after the 10 minutes ENPP1 and test compound incubation the assay was initiated by transferring 50 μl of the above mentioned ENPP1/test compound reaction into 50 μl of 1 mM pNP-TMP in assay buffer results in a 100 μl total reaction in clear bottom 96 well plates. Absorbance at 405 nm was recorded immediately in kinetic mode by PerkinElmer 2300 Enspire multimode plate reader.

For each inhibitor, the specific ENPP1 activity was calculated using the following equation: ENPP1 activity (pmol/min/μg)=Adjusted Vmax (OD405 nm/min)×conversion factor (pmol/OD405 nm)/amount of enzyme (μg)

Adjusted Vmax=V₀×(Km+(S))/(S). In this assay, Km=232 μM, (S)=500 μM. Adjusted Vmax=1.464×V₀.
V₀=(OD405 nm with ENPP1−OD405 nm ENPP1 blank)/minutes. OD405 nm was plotted, with blank subtracted, against time (minutes), the initial linear rate is V₀.
blank subtracted, against time (minutes), the initial linear rate is V₀.

The conversion factor (pmol/OD405 nm), was determined by plotting the amount of standard, 4-Nitrophenol (Sigma-Aldrich, Catalog #241326), against absorbance at 405 nm. The slope is the conversion factor. The percent ENPP1 activity for each sample was calculated using the following equation:

% enzyme activity=sample enzyme activity/MAX Activity×100%.

To determine the IC₅₀ for each compound, compound concentration values and percent enzyme activity values were inserted into GraphPad Prism (GraphPad Prism version 7.0 for Windows, GraphPad Software, La Jolla Calif. USA, www.graphpad.com), and Prism's Transform analysis was used to convert the x-axis values (compound concentration) to logarithms. A sigmoidal variable slope nonlinear regression analysis was performed using the following equation: Y=Bottom+(Top−Bottom)/(1+10\hat{ }((Log IC₅₀⁻X)*HillSlope)).

Kᵢ values for each compound were calculated from the observed IC₅₀ from GraphPad analysis using the Cheng-Prusoff equation: Kᵢ=IC₅₀/(1+(S)/K_{M}). (S) here is 500 μM and K_{M} is determined to be 232 μM.

Ki for a representative compound of Formula (I) in Compound Table 1 above is provided in Table 2 below:

TABLE 2
Ki (pNP-TMP)

| Group A (<1 nM) | Group B (<5 nM) | Group C (<10 nM) |
|---|---|---|
| Cpd. 17 | Cpds 5, 6, 19, 30, 41 | Cpd. 7, 11, 12, and 24 |
| in Compound Table 1 | in Compound Table 1 | in Compound Table 1 |

### Example 2

### Measurement of 2′3′-cGAMP Hydrolysis by ENPP1

ENPP1 catalyzes the hydrolysis of 2′3′-cGAMP into 5′-AMP and 5′-GMP, and hence the ENPP1 enzyme activity with 2′3′-cGAMP as substrate was monitored by measurement of the product 5′-AMP. The AMP-Glo assay kit from Promega (catalog number V5012) was used for measurement of 5′-AMP production.

First, an ENPP1 and test compound incubation was set up in assay buffer (50 mM Tris-HCl, pH8.8, 250 mM NaCl, 0.1 mg/ml BSA, 1% DMSO) with following conditions: ENPP1 concentration: 1.25 nM; test compound concentration ranging from 68 pM to 20 This incubation was carried out at 25° C. for 10 min.

Second, after the 10 minute ENPP1 and test compound incubation, prepare on a separate plate, 15 μl of the substrate 2′3′-cGAMP at 200 μM in assay buffer. Then, 15 μl of the ENPP1/Compound incubation was transferred to the 200 μM 2′3′-cGAMP solution to initiate the reaction. The 30 μl mixture was incubated for 30 min at 25° C. In all these assays a DMSO control without compound was included which gave the maximum 5′-AMP production (MAX RLU). After 30 min the reaction was stopped by heating at 90° C. for 3 min.

Third, now the Promega AMP-Glo kit was used to detect 5′-AMP production as a measurement of ENPP1 enzyme activity. To do this, 10 μl of the above mentioned 30 μl total reaction per sample was transferred into 384 well white solid assay plate for measurement of 5′-AMP production. For each well, 10 μl of AMP-Glo Reagent I was added, mixed well, and incubated for 1 hour at 25° C. At this time AMP detection solution was prepared and 20 μl was added per well, and the resulting solution was incubated for 1 hr at 25° C. Duplicates were run for each inhibitor concentration. Luminescence signal (relative luminescence units, RLU) was recorded using a PerkinElmer 2300 Enspire multimode plate reader.

The % inhibition was calculated using the following equation: % inhibition=(MAX RLU−sample RLU)/MAX RLU×100%.

IC₅₀ values of compounds were determined by loading compound concentration data and percent inhibition values into GraphPad Prism (GraphPad Prism version 7.0 for Windows, GraphPad Software, La Jolla Calif. USA, www.graphpad.com) and conducted a Sigmoidal variable slope nonlinear regression fitting.

Kᵢ values for each compound were calculated from the observed IC₅₀ from GraphPad analysis using the Cheng-Prusoff equation: Kᵢ=IC₅₀/(1+(S)/KM). (S) here is 100 μM and K_{M} is 32 μM. Ki for a representative compound of Formula (I) in Compound Table 1 above is provided in Table 3 below:

TABLE 3
Ki (2′3′-cGAMP)

| Group A (<1 nM) | Group B (<5 nM) | Group C (<10 nM) |
|---|---|---|
| Cpd. 3, 5, 6, 7, 12, | Cpd. 4, 13, 18 | Cpds. 8, 10, and 21 |
| and 19 | and 20 | in Compound Table 1 |
| in Compound Table 1 | In Compound Table 1 | |

### FORMULATION EXAMPLES

The following are representative pharmaceutical formulations containing a compound of the present disclosure.

### Tablet Formulation

The following ingredients are mixed intimately and pressed into single scored tablets.

| | Quantity per tablet |
|---|---|
| Ingredient | mg |
| compound of this disclosure | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per capsule |
|---|---|
| Ingredient | mg |
| compound of this disclosure | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 ul of spray for each application.

What is claimed:

1. A compound of Formula (IA): 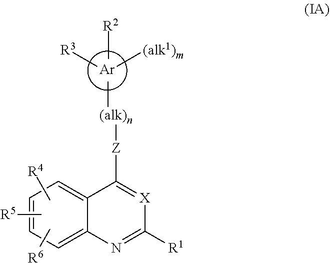
wherein:
X is N or CH;
Z is NH, O, S, SO, or SO₂;
alk is alkylene optionally substituted with one, two, or three halo;
m and n are independently 0 or 1;
(i) —Ar-(alk¹)ₘ-Q is wherein Ar is aryl, heteroaryl, cycloalkyl, or heterocyclyl;
alk¹ is alkylene wherein one carbon atom in the alkylene chain is optionally replaced by oxygen and further wherein alkylene is optionally substituted with one, two, or three halo; and
Q is —B(OH)₂ or —P(O)(R^{a})(R^{b}) wherein R^{a} and R^{b} are independently selected from hydroxy, alkoxy,-Oaryl (where aryl is optionally substituted with one to three substituents independently selected from alkyl, alkenyl, alkoxy, halo, haloalkyl, amino, alkylamino, dialkylamino, cyano, or nitro), —O—(CH₂)OCOR^{c} (where R^{c} is alkyl), —O-(alk²)OR^{d} (where alk² is alkylene and R^{d} is alkyl), —S—(CH₂)₂SCOR^{e} (where R^{e} is alkyl), or —NR^{g}—(CHR)OCOR^{f} (where R is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl either alone or as part of phenylalkyl is optionally substituted with one to three substituents independently selected from alkyl, alkoxy, halo, hydroxy, cyano or nitro), R^{f} is alkyl or benzyl, and R^{g} is hydrogen or R^{g} together with R forms —(CH₂)₃—); or R^{a} and R^{b} together with the phosphorus atom to which they are attached form a ring of formula (c): 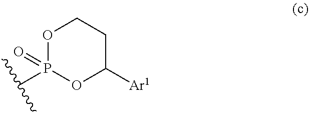
wherein Ar¹ is phenyl or six membered heteroaryl optionally substituted with one to three halo; provided that, when Q is —P(O)(R^{a})(R^{b}) or —B(OH)₂, then at least one of n and m is 1; or
(ii) —Ar-(alk¹)ₘ-Q is a ring of formula (a): 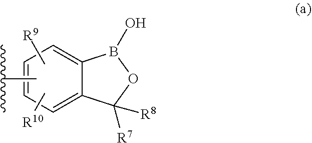
R¹ is hydrogen, alkyl, alkoxy, halo, haloalkyl, haloalkoxy, amino, alkylamino, dialkylamino, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, diaminoalkyl, diaminoalkoxy, diaminoalkylamino, or cyano;
R², R³, R⁹, and R¹⁰ are independently hydrogen, alkyl, alkoxy, halo, haloalkyl, or haloalkoxy, or cyano;
R⁷ and R⁸ are independently hydrogen or alkyl; and
one of R⁴, R⁵, and R⁶ is hydrogen, alkyl, hydroxy, alkoxy, halo, haloalkyl, haloalkoxy, cyano, amino, alkylamino, or dialkylamino; and the remaining two of R⁴, R⁵, and R⁶ are independently hydrogen, alkyl, alkoxy, hydroxy, halo, haloalkyl, haloalkoxy, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano); or
a pharmaceutically acceptable salt thereof.

2. The compound of claim 1 wherein the compound of Formula (IA) has a structure of formula (Ib1) or (Ic1): 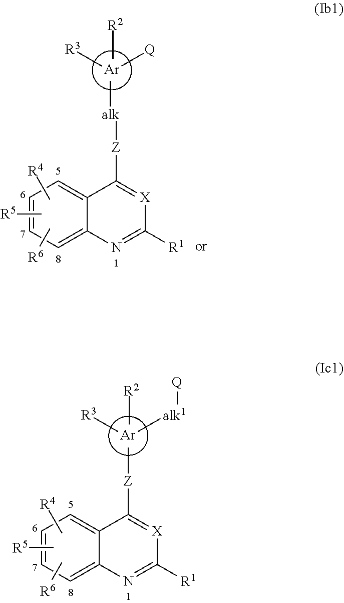
wherein Z is NH or O and Q is —B(OH)₂ or —P(O)(R^{a})(R^{b}).

3. The compound of claim 2, wherein the compound of Formula (IA) has a structure of formula (Ib1).

4. The compound of claim 2, wherein the compound of Formula (IA) has a structure of formula (Ic1).

5. A compound of claim 1, having a structure of Formula (Ib) or (Ic): 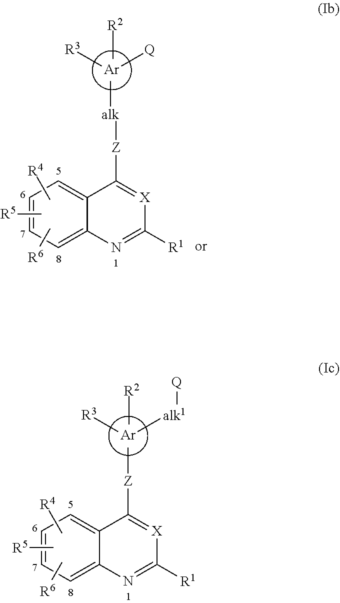
wherein
Z is NH or O;
alk is alkylene optionally substituted with one, two, or three halo;
m and n are independently 0 or 1;
—Ar-(alk¹)ₘ-Q is wherein Ar is aryl, heteroaryl, cycloalkyl, or heterocyclyl;
alk¹ is alkylene optionally substituted with one, two, or three halo; and
Q is —P(O)(OH)₂ or —B(OH)₂; provided that, when Q is —P(O)(OH)₂ or —B(OH)₂, then least one of n and m is 1;
R¹, R², and R³ are independently hydrogen, alkyl, alkoxy, halo, haloalkyl, haloalkoxy, or cyano; and
one of R⁴, R⁵, and R⁶ is hydrogen, alkyl, hydroxy, alkoxy, halo, haloalkyl, haloalkoxy, cyano, amino, alkylamino, or dialkylamino; and the remaining two of R⁴, R⁵, and R⁶ are independently hydrogen, alkyl, alkoxy, hydroxy, halo, haloalkyl, haloalkoxy, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano); or
a pharmaceutically acceptable salt thereof.

6. The compound of claim 5, wherein the compound has a structure of formula (Ib).

7. The compound of claim 5, wherein the compound has a structure of formula (Ic).

8. The compound of any one of claims 1 to 4 wherein Z is NH.

9. The compound of any one of claims 5 to 7 wherein Z is NH.

10. The compound of any one of claims 1 to 4 wherein Z is O.

11. The compound of any one of claims 5 to 7 wherein Z is O.

12. The compound of any one of claims 1 to 4, 8, and 10 wherein Ar is phenyl.

13. The compound of any one of claims 5 to 7, 9 and 11 wherein Ar is phenyl.

14. The compound of claim 12 wherein Q is attached to the carbon of the phenyl ring that is meta or para to the carbon attaching the phenyl ring to the remainder of the compound of Formula (IA), (Ib1), or (Ic1).

15. The compound of claim 13 wherein Q is attached to the carbon of the phenyl ring that is meta or para to the carbon attaching the phenyl ring to the remainder of the compound of Formula (Ib) or (Ic).

16. The compound of any one of claims 1 to 4, 8, and 10 wherein Ar is heteroaryl.

17. The compound of any one of claims 5 to 7, 9 and 11 wherein Ar is heteroaryl.

18. The compound of claim 16 wherein heteroaryl is a six membered ring and Q is attached to the carbon of the heteroaryl ring that is meta or para to the carbon of the heteroaryl ring attaching the heteroaryl ring to remaining compound of Formula (IA), (Ib1), or (Ic1).

19. The compound of claim 17 wherein heteroaryl is a six membered ring and Q is attached to the carbon of the heteroaryl ring that is meta or para to the carbon of the heteroaryl ring attaching the heteroaryl ring to remaining compound of Formula (Ib) or (Ic).

20. The compound of claim 1, 4, or 5 wherein Ar is heterocyclyl.

21. The compound of claims 5 to 7 wherein Ar is heterocyclyl.

22. The compound of any one of claims 1 to 4, 8, 10, 12, 14, 16, and 18 wherein alk and alk¹ are independently methyl, ethyl, or propyl.

23. The compound of claim 22, wherein alk and alk¹ are methyl.

24. The compound of any one of claims 5 to 7, 9, 11, 13, 15, 17, and 19 wherein alk and alk¹ are independently methyl, ethyl, or propyl.

25. The compound of claim 24, wherein alk and alk′ are methyl.

26. The compound of any one of claims 1 to 4, 8, 10, 12, 14, 16, 18, 22 and 23, wherein R² and R³, R⁹, and R¹⁰ are independently hydrogen, methyl, ethyl, methoxy, fluoro, trifluoromethyl, trifluoromethoxy, or cyano.

27. The compound of any one of claims 5 to 7, 9, 11, 13, 15, 17, 19, 24, and 25 wherein R² and R³ are independently hydrogen, methyl, ethyl, methoxy, fluoro, trifluoromethyl, trifluoromethoxy, or cyano.

28. The compound of any one of claims 1 to 4, 8, 10, 12, 14, 16, 18, 22, 23, and 26 wherein R⁴ is hydrogen, methyl, hydroxy, methoxy, ethoxy, fluoro, chloro, trifluoromethyl, cyano, or trifluoromethyloxy.

29. The compound of claim 28 wherein R⁴ is hydrogen.

30. The compound of any one of claims 5 to 7, 9, 11, 13, 15, 17, 19, 25, and 27 wherein R⁴ is hydrogen, methyl, hydroxy, methoxy, ethoxy, fluoro, chloro, trifluoromethyl, cyano, or trifluoromethyloxy; preferably R⁴ is hydrogen, methoxy or ethoxy, more preferably R⁴ is hydrogen.

31. The compound of claim 30 wherein R⁴ is hydrogen.

32. The compound of any one of claims 1 to 4, 8, 10, 12, 14, 16, 18, 22, 23, 26, 28, and 29 wherein R⁵ and R⁶ are independently hydrogen, alkyl, alkoxy, hydroxy, halo, haloalkyl, or haloalkoxy.

33. The compound of claim 32 wherein R⁵ and R⁶ are independently methoxy, ethoxy, or propoxy and are attached to C6 and C7 carbons of the bicyclic ring.

34. The compound of any one of claims 5 to 7, 9, 11, 13, 15, 17, 19, 24, 25, 27, 30 and 31 wherein R⁵ and R⁶ are independently hydrogen, alkyl, alkoxy, hydroxy, halo, haloalkyl, or haloalkoxy.

35. The compound of claim 34, wherein R⁵ and R⁶ are independently methoxy, ethoxy, or propoxy and are attached to C6 and C7 carbons of the bicyclic ring.

36. The compound of any one of claims 1 to 4, 8, 10, 12, 14, 16, 18, 22, 23, 26, 28, 29, 32, and 33 wherein R¹ is hydrogen, alkyl, alkoxy, halo, haloalkyl, haloalkoxy, amino, alkylamino, dialkylamino, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, diaminoalkyl, diaminoalkoxy, diaminoalkylamino, or cyano.

37. The compound of any one of claims 5 to 7, 8, 10, 13, 15, 17, 19, 24, 25, 27, 30, 31, 34, and 35 wherein R¹ is hydrogen or methyl.

38. The compound of claim 37 wherein R¹ is hydrogen.

39. The compound of any one of claims 1 to 4, 8, 10, 12, 14, 16, 18, 22, 23, 26, 28, 29, 32, 33, and 36 wherein Q is —B(OH)₂.

40. The compound of any one of claims 5 to 7, 8, 10, 13, 15, 17, 19, 24, 25, 27, 30, 31, 34, 35, 37 and 38 wherein Q is —B(OH)₂.

41. The compound of any one of claims 1 to 4, 8, 10, 12, 14, 16, 18, 22, 23, 26, 28, 29, 32, 33 and 36 wherein Q is —P(O)(R^{a})(R^{b}).

42. The compound of any one of claims 5 to 7, 8, 10, 13, 15, 17, 19, 24, 25, 27, 30, 31, 34, 35, 37 and 38 wherein Q is —P(O)(OH)₂.

43. The compound of any one of claims 1 to 4, 8, 10, 12, 14, 16, 18, 22, 23, 26, 28, 29, 32, 33, 36, 39, and 41 wherein R¹ is hydrogen or alkyl.

44. The compound of claim 43 wherein R¹ is hydrogen.

45. The compound of any one of claims 1 to 4, 8, 10, 12, 14, 16, 18, 22, 23, 26, 28, 29, 32, 33, 36, 39, and 41 wherein R¹ is halo, haloalkyl, or haloalkoxy.

46. The compound of any one of claims 1 to 4, 8, 10, 12, 14, 16, 18, 22, 23, 26, 28, 29, 32, 33, 36, 39, and 41 wherein R¹ is amino, alkylamino, or dialkylamino.

47. The compound of any one of claims 1 to 4, 8, 10, 12, 14, 16, 18, 22, 23, 26, 28, 29, 32, 33, 36, 41, and 43 to 46 wherein R^{a} and R^{b} are independently hydroxy, alkoxy,-Ophenyl (where phenyl is optionally substituted with one to three substituents independently selected from alkoxy, halo, haloalkyl, cyano, or nitro), —O—(CH₂)OCOR^{c} (where R^{c} is alkyl), or —NH—(CHR)OCOR^{f} (where R is alkyl, R^{f} is alkyl or benzyl).

48. The compound of claim 47 wherein R^{a} and R^{b} are hydroxy.

49. A pharmaceutical composition comprising a compound any one of claims 1 to 47 and a pharmaceutically acceptable excipient.

50. A method of treating a disease or condition modulated at least in part by ENPP1 in a patient comprising administering to the patient a compound of any one of claims 1 to 47.

51. The method of claim 50 wherein the disease or condition is a cancer, an inflammatory disease, a metabolic disease, or a viral disease.

52. The method claim 50 wherein the disease or condition is a cancer wherein the cancer is hepatocellular carcinomas, glioblastomas, melanomas, testicular, pancreatic, thyroid or breast cancer.
